(19) United States
(12) Patent Application Publication
(10) Pub. No.: US 2004/0191842 A1
(43) Pub. Date: Sep. 30, 2004

(54) Chemopreventive and therapeutic aspects of polyphenolic compositions and assays

(75) Inventors: Stephen Hsu, Evans, GA (US); George Schuster, Augusta, GA (US); Jill Lewis, Martinez, GA (US); Baldev Singh, Augusta, GA (US); Fu-Shin Yu, Martinez, GA (US)

Correspondence Address: MUETING, RAASCH & GEBHARDT, P.A., P.O. BOX 581415, MINNEAPOLIS, MN, 55458, US

(73) Assignee: Medical College of Georgia Research Institute, Inc., Augusta, GA

(21) Appl. No.: 10/732,782

(22) Filed: Dec. 10, 2003

Related U.S. Application Data
(60) Provisional application No. 60/432,086, filed on Dec. 10, 2002.

(30) Foreign Application Priority Data
Dec. 10, 2003 (WO) PCT/US03/39302

(51) Int. Cl. G01N 33/574

(52) U.S. Cl. 435/7.23

(57) ABSTRACT

The present invention includes chemopreventive and therapeutic methods based on the administration of polyphenolic compositions, including the polyphenolic compositions found in green tea. The present invention also includes various screening assay for the identification of chemopreventive and therapeutic agents.

### CONTINUING APPLICATION DATA

This application claims the benefit of U.S. Provisional Application Serial No. 60/432086, filed Dec. 10, 2002, which is incorporated by reference herein.

### GOVERNMENT FUNDING

The present invention was made with government support under Grant No. CA097258-01A1, awarded by the National Cancer Institute, National Institutes of Health. The Government may have certain rights in this invention.

### BACKGROUND

Cancer is the second leading cause of death in the United States, second only to cardiovascular diseases, with the incidence of oral cancer approximately 2-6% of all cancers. In the United States, more than 30,000 patients will be diagnosed with oral cancer with an estimated 7800 deaths, with a rather static five-year mortality rate of 53% to 56% reported for the past few years. Furthermore, the disfigurement from surgical treatment of these cancers often results in prolonged trauma in patients even after the disease has been controlled. The highest rates of oral cancer are in developing countries, such as South Asia and Southeast Asia, where oral cancer is the first or second most common malignancy. In some parts of India oral cancer accounts for 30-40% of all cancers and is regarded as a ‘new epidemic.’

The risk factors for oral squamous cell carcinoma include smoking, such as cigarettes, cigars, and pipes, the use of smokeless tobacco, such as chewing tobacco and snuff, and drinking alcohol, which has a synergistic effect with smoking. The differential oral cancer incidence among countries, and even among populations in the same country may reflect variations in the etiologic factors, tumor promoters and their interaction with dietary constituents, habits, genetics, environment, and hygiene. It is evident that smoking is one of the etiological factors in the development of oral cancer. However, there is still no explanation as to why China, which has a population of heavy smokers and poor oral hygiene, has a far lowest incidence of oral cavity, lip, and pharyngeal cancers in males (4.66 per 100,000) compared to North America (11.69 per 100,000) and South Central Asia (20.5 per 100,000) (Parkin et al., CA Cancer J Clin 1999; 49: 33-64, 2, Parkin et al., International of Cancer 1993:54:594-606). The Chinese population is unique in its high consumption of green tea.

Thus, there exits a need for improved methods of preventing and treating oral cancers. There also exits a need for improved methods of drug screening to identify new agents effective for the prevention and or treatment of cancer, including oral cancers.

### SUMMARY OF THE INVENTION

The present invention includes a method of determining if cancer cells are resistant to an agent, the method including determining the p57/KIP2 level in the cancer cells prior to contact with the agent; contacting the cancer cells with the agent; determining the p57/KIP2 level in the cancer cells after contact with the agent; and comparing the p57/KIP2 level in the cancer cells after contact with the agent to the p57/KIP2 level in the cancer cells prior to contact with the agent; wherein an increase in the p57/KIP2 level in the cancer cells after contact with the agent compared to the p57/KIP2 level in the cancer cells prior to contact with the agent indicates the cancer cells are resistant to the agent.

The present invention also includes a method of determining if cancer cells are sensitive to an agent, the method including determining the p57/KIP2 level in the cancer cells prior to contact with the agent; contacting the cancer cells with the agent; determining the p57/KIP2 level in the cancer cells after contact with the agent; and comparing the p57/KIP2 level in the cancer cells after contact with the agent to the p57/KIP2 level in the cancer cells prior to contact with the agent; wherein no increase in the p57/KIP2 level in the cancer cells after contact with the agent compared to the p57/KIP2 levels in the cancer cells prior to contact with the agent indicates the cancer cells are sensitive to the agent.

The present invention also includes a method of identifying an agent effective for the treatment of a cancer, the method including determining the p57/KIP2 level in cancer cells prior to contacting with the agent; contacting the cancer cells with the agent; determining the p57/KIP2 level in the cancer cells after contacting with the agent; and comparing the p57/KIP2 level in the cancer cells after contacting with the agent to the p57/KIP2 level in the cancer cells prior to contacting with the agent; wherein no increase in the p57/KIP2 level in the cancer cells after contacting with the agent compared to the p57/KIP2 level in the cancer cells prior to contacting with the agent indicates the agent is effective for the treatment of a cancer.

The present invention also includes a method of determining the therapeutic effectiveness of an agent, the method including contacting normal cells with the agent; determining the p57/KIP2 level in the normal cells after contacting with the agent; contacting cancer cells with the agent; determining the p57/KIP2 level in the cancer cells after contacting with the agent; and comparing the p57/KIP2 level in the normal cells after contacting with the agent to the p57/KIP2 level in the cancer cells after contacting with the agent; wherein a higher p57/KIP2 level in the normal cells compared to the p57/KIP2 level in the cancer cells indicates the agent is effective for the treatment of cancer. In some embodiments, the normal cells and cancer cells are cultured together.

The present invention also includes a method of optimizing the formulation of an agent for the treatment of a cancer, the method including contacting cancer cells with a first formulation of the agent; determining the p57/KIP2 level in the cancer cells contacted with the first formulation of the agent; contacting cancer cells with a second formulation of the agent; determining the p57/KIP2 level in the cancer cells contacted with the second formulation of the agent; and comparing the p57/KIP2 level in the cancer cells contacted with the first formulation of the agent to the p57/KIP2 level in the cancer cells contacted with the second formulation of the agent; wherein the formulation with the lower level of p57/KIP2 indicates the formulation of the agent more effective for the treatment of a cancer.

The present invention also includes a method of preventing damage to non-cancerous cells in a subject undergoing cancer therapy, the method including administering to the subject a polyphenolic composition under conditions effective to induce the expression of p57, induce the expression of caspase-14, or induce the expression of both p57 and caspase-14 in non-cancerous cells.

The present invention also includes a method of enhancing the effectiveness of a cancer therapy in a subject undergoing cancer therapy, the method including administering to the subject a polyphenolic composition under conditions effective to induce caspase 3-dependent apoptosis in cancer cells.

The present invention also includes a method of preventing damage to salivary glands cells in a subject undergoing therapy for oral cancer, the method including administering to the subject a polyphenolic composition under conditions effective to induce the expression of p57, induce the expression of caspase-14, or induce the expression of both p57 and caspase-14.

The present invention also includes a method of treating a skin condition, the method including contacting the skin with a polyphenolic composition under conditions effective to induce caspase-14 expression in keratinocytes. In some embodiments, the skin condition may be psoriasis, aphthous ulcer, actinic keratosis, rosacea, a wound, a burn, a skin condition associated with diabetes, a skin condition associated with aging, or a skin condition associated with altered keratinocyte differentiation.

The present invention also includes a method of treating a precancerous oral lesion, the method including contacting the precancerous oral lesion with a polyphenolic composition under conditions effective to induce p57 expression in normal epithelial cells and induce caspase 3-dependent apoptosis in precancerous and cancerous epithelial cells.

The present invention also includes an in vitro method for the identification of an agent that possesses both a cytotoxic effect on tumor cells and a protective effect on normal cells, the method including co-culturing normal cells adjacent to tumor cells in vitro; contacting the co-cultured cells with an agent; determining if contact with the agent induces tumor cell death; and determining if normal cells survive upon contact with the agent; wherein the induction of tumor cell death by contact with the agent and the survival of normal cells upon contact with the agent indicated the agent possesses both a cytotoxic effect on tumor cells and a protective effect on normal cells. In some embodiments, both the tumor cells and normal cells are of epithelial origin. In some embodiments, both the tumor cells and normal cells are human cells. In some embodiments, the induction of tumor cell death upon contact with an agent is determined by detecting apoptosis of the tumor cell. In some embodiments, the tumor cells are a tumor cell line stably transfected with green fluorescent protein (GFP), including the human oral carcinoma cell line OSC-2 stably transfected with GFP. In some embodiments, survival of normal cells upon contact with an agent is determined by detecting the induction of p57 expression in the normal cells.

The present invention includes agents identified by the methods of the present invention.

The present invention includes a kit for the identification of an agent that possesses both a cytotoxic effect on tumor cells and a protective effect on normal cells, the kit including normal cells, tumor cells transfected with green fluorescent protein (GFP), and printed instructions for the identification of an agent that possesses both a cytotoxic effect on tumor cells and a protective effect on normal cells.

In some embodiments of the methods of the present invention the polyphenolic composition is green tea polyphenol (GTPP), (−)-epicatechin (EC), (−)-epigallocatechin (EGC), (−)-epicatechin-3-gallate (ECG), (−)-epigallocatechin-3-gallate (EGCG), or combinations thereof.

In some embodiments of the methods of the present invention, determining the p57/KIP2 level is by detecting the p57/KIP2 protein.

In some embodiments of the methods of the present invention, determining the p57/KIP2 level is by detecting the mRNA encoding p57/KIP2.

In some embodiments of the methods of the present invention, the cancer cell is an epithelial carcinoma cell line, including, for example, an oral squamous carcinoma cell line, a metastatic oral carcinoma cell line, or a breast epithelial carcinoma cell line.

In some embodiments of the methods of the present invention, the cancer cells are derived from a human epithelial carcinoma, including human epithelial carcinomas selected from an oral squamous carcinoma, a metastatic oral carcinoma, or a breast epithelial carcinoma.

In some embodiments of the methods of the present invention, the cancer is oral cancer, esophageal cancer, gastric cancer, colorectal cancer, prostate cancer, bladder cancer, skin cancer, or cervical cancer.

In some embodiments of the methods of the present invention, the polyphenolic composition is administered to the subject prior to, coincident with, or subsequent to the cancer therapy. Such a cancer therapy may be, for example, chemotherapy, radiation therapy, or a combination thereof.

Definitions

As used herein, a “subject” is an organism, including, for example, an animal. An animal includes, but is not limited to, a human, a non-human primate, a horse, a pig, a goat, a cow, a rodent, such as, but not limited to, a rat or a mouse, or a domestic pet, such as, but not limited to, a dog or a cat. Subject also includes model organisms, including, for example, animal models, used to study tumor progression, growth, or metastasis, or to study wound healing.

A “control” sample or subject is one that has not been treated with a polyphenolic composition.

As used herein in vitro is in cell culture, ex vivo is a cell that has been removed from the body of a subject and in vivo is within the body of a subject.

As used herein, “treatment” or “treating” include both therapeutic and prophylactic treatments.

Unless otherwise specified, “a,” “an,” “the,” and “at least one” are used interchangeably and mean one or more than one.

### BRIEF DESCRIPTION OF THE FIGURES

FIGS. 1A and 1B represent Western blot analysis of whole cell lysates. FIG. 1A represents differential p57-induction demonstrated by Western blot analysis of whole cell lysates from human keratinocytes, SCC25, and OSC2 human oral carcinoma cells at 40% confluency. Only the keratinocytes responded to (−)-epigallocatechin-3-gallate (EGCG) and GTPPs by elevation of p57. Cells were treated for 24 hours as follows: control (C); 50 μM EGCG (E); 0.2 mg/ml GTPPs (G). p57 levels in SCC25 and OSC2 cells remained unchanged. FIG. 1B represents p57 induction in Western blot analysis of whole cell lysates from human keratinocytes treated under different conditions: Control (1); BaP (2); NNK (3); EGCG (4); EGCG+BaP (5); and EGCG+NNK (6). The relative densities of the p57 bands were compared on Western blots using the UTHSCSA Image Tool imaging software. The blot image was converted from color to grayscale and the band density measured on a scale of 1-255 densitometric units/mm². Lane 4 (EGCG treated) represents a 12-fold increase comparing to lane1 (Control).

FIGS. 2A and 2B represent Western blot analysis of whole cell lysates from human keratinocytes. FIG. 2A represents reversible p57 induction in Western blot analysis of whole cell lysates from human keratinocytes in a time course experiment with 85-90% cell density. Lane 1 is 24 hours untreated cells as control (C); Lanes 2-5 are EGCG cells treated for the indicated length of time with 50 μM EGCG; EGCG+Chx. Lanes 6-9 are cells treated for the indicated length of time with 50 μM EGCG and 30 μg/ml cycloheximide. FIG. 2B represents increasing p57 expression in Western blot analysis of whole cell lysates from human keratinocytes on dose response experiment with 85-90% cell density. Cells were treated with indicated concentrations for 24 hours prior to harvesting.

FIG. 3 shows lack of p57-induction demonstrated by Western analysis of whole cell lysates from OSC2 cells. Darker background with p57 bands was due to extended exposure time following ECL reaction (10 minutes). Lanes 1-4 show samples treated with indicated concentration of EGCG for 24 hours. Lane 5 (C) contains control sample without any treatment, lanes 6-9 contain samples treated with 50 μM EGCG for the indicated length of time. The nitrocellulose membrane was hybridized with anti-p57 antibody followed by hybridization with anti-human actin antibody.

FIGS. 4A and 4B summarize inhibition of growth and invasiveness of OSC2 cells by EGCG treatment. FIG. 4A shows growth inhibition of OSC2 cells by EGCG. OSC2 cells were incubated with 50 μM EGCG for 24, 48, and 96 hours, and cell number were counted in comparison with the cell number of untreated control. FIG. 4B shows inhibition of invasiveness of OSC2 cells by EGCG treatment. After 24, 48, 96 hours of treatments with EGCG, cells (10⁵) were loaded onto each transwell of a 24-well transwell plate. Both tests were conducted three times with similar results. The controls are presented as 100% in cell number.

FIG. 5 represents a schematic model for the dual-effects of green tea polyphenols, that differentially target between normal and tumor cells. Either survival pathway or apoptotic pathway could be activated, depending on whether p57 protein production is induced. Induction of p57 appears to inhibit the apoptotic pathway. C3 represents caspase 3.

FIGS. 6A and 6B present results of treatment of mammary epithelial cells with increasing concentrations of EGCG. FIG. 6A shows western blot of whole cell lysates from mammary epithelial cells exhibiting up-regulation of Apaf-1 levels and basal p57 levels when treated with increasing concentrations of EGCG. FIG. 6B shows results of caspase 3 activity assay performed on the same cells. Detection of caspase 3 activities was based on PARP cleavage by caspase 3. EGCG concentration ranged from 0 to 200 μM. Experiments were repeated three times with similar results. Each bar represents average of triplicate samples and SD.

FIGS. 7A and 7B present results of treatment of human epidermal epithelial cells with increasing concentrations of EGCG. FIG. 7A shows Western blot analysis of whole cell lysates from human epidermal epithelial cells with EGCG treatments as indicated. No significant changes shown in Apaf-1 bands or PCNA bands measured by densitometry, compared to actin levels. FIG. 7B shows results of caspase 3 activity assay performed on the same cells. No elevation of caspase 3 activity was recorded. EGCG concentration ranged from 0 to 200 μM. “G” is 0.2 mg/ml GTPPs. Experiments were repeated three times with similar results. Each bar represents average of triplicate samples and SD.

FIGS. 8A through 8D present caspase 3 activity assay results showing elevated caspase 3 activities in MCF7(C) cells (FIG. 8A) in comparison with MCF7 cells (FIG. 8B). MCF7(C) cells responded to increasing concentrations of EGCG and 0.2 mg/ml GTPPs in a 24-hour period similarly to OSC2 cells (FIG. 8C), a well-characterized oral squamous cell carcinoma cell line that undergoes apoptosis when exposed to GTPPs. Both cell lines exhibited highest levels of caspase 3 activities in response to 0.2 mg/ml GTPPs. The caspase 3 null MCF7 cells responded to identical treatment similarly to normal human epidermal keratinocytes, which also failed to elevate caspase 3 activities (FIG. 8D). Experiments were repeated three times. Each column represents the average of triplicate samples and SD.

FIGS. 9A and 9B present 5-bromo-2-deoxyuridine (BrdU) incorporation assay results showing OSC2 oral carcinoma cells ceased BrdU incorporation when exposed to EGCG concentrations greater than 50 μM or to GTPPs (GTP) (FIG. 9A). Under identical conditions, the caspase 3 null MCF7 cells exhibited normal levels of BrdU incorporation compared to control with sight decrease when exposed to GTPPs (FIG. 9B). Experiments were repeated for three times with similar patterns. Each column represents the average of triplicate samples and SD.

FIGS. 10A and 10B present cell growth assay and MTT (3-(4,5-dimethylthiazol-2-yl)-2,5-diphenyl tetrazolium bromide) assay for MCF7 cells.

In FIG. 10A caspase 3 null MCF7 cells did not show significant growth inhibition by 50 μM EGCG when the cells were cultured for the indicated time periods. Each column represents the average of triplicate samples and SD. In FIG. 10B MCF7 cells showed significant loss in mitochondrial SDH activities when treated with 50 μM EGCG or 0.2 mg/ml GTP for the indicated time periods. Each column represents the average of triplicate samples and SD.

FIGS. 11A and 11B show MTT assay results for OSC2 and MCF7 cells. MTT assays results showing decreasing mitochondrial SDH activities are associated with increasing concentrations of EGCG as indicated or 0.2 mg/ml GTPPs (GTP) in both OSC2 cells (FIG. 11A) and caspase 3 null MCF7 cells (FIG. 11B). Both cell lines exhibited lowest SDH activities when exposed 0.2 mg/ml GTPPs for 24 hours. Experiments were repeated three times with similar patterns. Each column represents the average of triplicate samples and SD.

FIGS. 12A through 12F show EGCG and GTPPs stimulate mitochondrial energy production and DNA synthesis in aged keratinocytes. FIG. 12A, FIG. 12C, and FIG. 12E show MTT assay results of normal human primary epidermal keratinocytes cultured for 15 days, 20 days, or 25 days in KGM-2 medium, respectively, and treated with increasing concentrations of EGCG as indicated, or 0.2 mg/ml GTPPs for 24 hours. Data represent the average and standard deviation of triplicate samples. Experiments were repeated five times with consistent results. FIG. 12B, FIG. 12D, and FIG. 12F show BrdU assay results of normal human primary epidermal keratinocytes cultured for 15 days, 20 days, and 25 days in KGM-2 medium, respectively, and treated with increasing concentrations of EGCG as indicated or 0.2 mg/ml GTP for 24 hours. Data represent the average and standard deviation of triplicate samples. Experiments were repeated three times with consistent results, and the above experiments were performed in parallel.

FIG. 13 shows that EGCG and GTPPs stimulate transglutaminase activity in exponentially growing keratinocytes. Comparison of transglutaminase activity (a late differentiation marker) between control and EGCG-treated cells. Cells treated with 50 μM or 100 μM EGCG have significantly higher activity. Data represent the average and standard deviation of triplicate samples. Experiments were repeated three times with similar results.

FIGS. 14A through 14C show EGCG and GTPPs exert minimal effects on DNA synthesis and do not alter mitochondrial energy production or apoptosis in exponentially growing keratinocytes. Exponentially growing normal human primary epidermal keratinocytes were evaluated for DNA synthesis, caspase 3 activities and SDH activities following treatment with increasing concentrations of EGCG as indicated or 0.2 mg/ml GTP. The results of the BrdU assay showed a slight increase of BrdU incorporation (FIG. 14A), while the caspase 3 assay (FIG. 14B) and MTT assay (FIG. 14C) were not significantly affected. Data represent the average with SD of triplicate samples. All experiments were performed three times with similar results.

FIGS. 15A through 15C demonstrate differential responses in intracellular ROS production in oral squamous cell carcinoma cells and normal epidermal keratinocytes. In FIG. 15A OSC-2 cells were treated with 50 μM, 200 μM of EGCG or 5 mM diamide, and the intracellular ROS levels were determined at the time points indicated, with untreated cells as control. In FIG. 15B OSC-4 cells underwent identical treatment and ROS levels were recorded as in FIG. 15A. In FIG. 15C normal human primary epidermal keratinocytes (NHEK) were treated identically as in OSC-2 and OSC-4 cells followed by ROS determination. Other concentrations of EGCG (15, 30, or 100 μM) produced identical results in NHEK as in FIG. 15C. Error bars indicate one standard deviation of the mean (n=3). Letters at 60 minutes denote statistical groupings (ANOVA, Tukey, α=0.05).

FIG. 16 shows intracellular ROS level determination in NS-SV-AC cells treated with various concentrations of EGCG for 60 minutes. Experiments were repeated three times with similar patterns. Error bars represent standard deviations (n=3). Letters denote statistical groupings (ANOVA, Tukey, α=0.05).

FIG. 17 shows intracellular catalase activities in cells treated with EGCG compared with untreated cells. Data presented as catalase activities versus cell numbers. Each experiment determined the activities of catalase in all three cell types in a single plate after incubation with 50 μM EGCG for 30 minutes. Experiments were repeated three times. Error bars represent standard deviations (n=3). Letters in each series denote statistical groupings (ANOVA, Tukey, (α=0.05). NS denotes no statistical different between controls and EGCG-treated cells (t-test, 2 sided, α=0.05).

FIG. 18 shows total SOD activities determined in cell lysates from three cell types treated with EGCG in comparison to untreated controls. Experiments were repeated three times with similar results. Each experiment tested the SOD activities versus cell numbers in three cell types in a single plate after incubation with 50 μM EGCG for 30 minutes. Error bars represent standard deviations (n=3). Letters in each series denote statistical groupings (ANOVA, Tukey, α=0.05). NS denotes no statistical different between controls and EGCG-treated cells (t-test, 2 sided, α=0.05).

FIGS. 19A and 19B show a comparison of MTT assay results and BrdU incorporation rates in OSC-2 cells and OSC-4 cells following EGCG treatment for 24 hours. Data presented as percentage of control. In FIG. 19A OSC-2 cells demonstrated higher sensitivity to EGCG in mitochondrial tricarboxylic acid cycle enzyme SDH than OSC-4 cells. In FIG. 19B OSC-2 cells were even more sensitive in BrdU incorporation, a measurement of new DNA synthesis, than OSC-4 cells. Experiments were repeated three times. Error bars represent standard deviations (n=3). Letters in each series denote statistical groupings (ANOVA, Tukey, α=0.05).

FIG. 20 represents survival and apoptotic pathways activated by GTPPs/EGCG. GTPPs or EGCG activate separate pathways dependant upon cell type. In normal human epithelial cells such as NHEK, EGCG induces p57 expression, followed by induction of keratins, fillagrin and caspase 14 (a terminal differentiation factor), and inhibition of p21 expression (cyclin dependent kinase that involves in growth arrest, apoptosis and differentiation), results in differentiation-associated cell survival (left). In many tumor cells, including OSC-GFP, a death signal is sent to the mitochondria, causing cytochrome c release and p21 expression, followed by the assembly of apoptosome and activation of the caspase cascade, results in apoptosis. In this case, the apoptosis is associated with the loss of fluorescence (right).

FIG. 21 demonstrates procedures involved in different designs for co-cultures. The overlay design requires two rounds of loading of cells, cells loaded in the second round cover the cells loaded in the first round (left). The adjacent design also requires two rounds of cell loading, but the two cell types are separated by a cylinder (right). After the co-cultures are treated with EGCG, the slides are subjected to fixation and immunofluorescence, followed by rhodamine and GFP detection and calculation.

FIG. 22 presents time-dependent EGCG-regulation of mRNA levels of caspase 14 and p21/WAF1. Open circle represent relative caspase 14 transcription after exposure to 100 μM EGCG for 0, 2, 6, and 24 hours (untreated control=1). Solid squares represent p21/WAF1 gene expression after 100 μM EGCG treatment, compared to untreated control. Two independent experiments were performed with similar results.

FIG. 23 presents EGCG-modulated protein changes in p21 and caspase 14 in NHEK. Western blots of whole cell lysates from NHEK treated for 0, 24, or 48 hours with 0-200 μM EGCG or for 30 minutes, 2 hours, or 6 hours with 50 μM EGCG. “C” represents control without treatment. EGCG concentrations were 15-200 μM. Bars indicate ratio of protein density to actin density. Data shown represents one of three independent Western blot analyses with similar results. Cell lysates from NHEK treated with 100 μM EGCG for 30 minutes, 2 hours, or 6 hours exhibited similar patterns to those treated by 50 μM EGCG at these time points.

FIGS. 24A and 24B represent mitochondrial succinate dehydrogenase (SDH) activities in NHEK, OSC-2, and OSC-4 cells following treatment with EGCG or H₂O₂. Cells were incubated with the indicated concentrations of EGCG (FIG. 24A) or H₂O₂ (FIG. 24B) for 24 hours, followed by MTT assay. These figures are representative of three experimental replications, all with similar results. Data are expressed as percentage of untreated cells, and error bars represent one standard deviation of the mean. Different capital letters indicate statistically significant differences among cell types (ANOVA, Tukey post-hoc test, α=0.05, n=3).

FIG. 25 represents intracellular ROS formation in OSC-2 and OSC-4 cells exposed to H₂O₂ (25-200 μM), EGCG (50 and 200 μM) and diamide (Di, 5 mM). Cells were incubated with or without these agents for 60 minutes, and intracellular ROS levels were determined by the DFDA assay. ROS levels are represented by relative fluorescence units (RFU). The figure is a representative experiment repeated three times with similar results. Error bars indicate one standard deviation of the mean. Different capital letters indicate statistically significant differences among conditions (ANOVA, Tukey post-hoc test, α=0.05, n=3).

FIG. 26 shows the influence of catalase and 3-AT (a catalase inhibitor) on EGCG-induced mitochondrial SDH activity reduction in OSC-2 and OSC-4 cells. Cells were either pretreated with 200 U/ml catalase for 5 minutes, or 30 μM 3-AT for 2 hours, prior to a 24-hour incubation with EGCG at concentrations indicated, immediately followed by MTT assay. These figures are representative of three experimental replications, all with similar results. Data are expressed as percentage of untreated cells. Error bars indicate one standard deviation of the mean. Different capital letters indicate statistically significant differences among conditions (ANOVA, Tukey post-hoc test, α=0.05, n=6).

FIGS. 27A and 27B represent mitochondrial succinate dehydrogenase (SDH) activity in OSC-2 and OSC-4 cells pretreated with N-acetyl cysteine (NAC) followed by incubation with either H₂O₂ or EGCG. OSC-2 and OSC-4 cells were pretreated with or without 10 mM NAC for 2 hours prior to incubation with the indicated concentrations of H₂O₂ (FIG. 27A) or EGCG (FIG. 27B) prior to MTT assay. These figures are representative of three experimental replications, all with similar results. Data are expressed as percentage of untreated cells. Error bars indicate one standard deviation of the mean. Different capital letters indicate statistically significant differences among conditions (ANOVA, Tukey post-hoc test, α=0.05, n=4).

FIG. 28A and 28B represent caspase-3 activity in OSC-2 and OSC-4 cells pretreated with catalase and incubated with EGCG. FIG. 28A represents OSC-2 cells. FIG. 28B represents OSC-4 cells. Cells were pretreated with 200 U/ml exogenous catalase for 5 minutes prior to addition of EGCG at concentrations indicated. Caspase-3 activity assay was performed immediately after 24 hours incubation with EGCG. Error bars indicate one standard deviation of the mean. Different capital letters indicate statistically significant differences among conditions (ANOVA, Tukey post-hoc test, α=0.05, n=4).

FIG. 29A and 29B represent BrdU incorporation in OSC-2 and OSC-4 cells following EGCG exposure with exogenous catalase. FIG. 29A represents OSC-2 cells. FIG. 29B represents OSC-4 cells. Cells were pretreated with or without 200 U/ml catalase for 5 minutes prior to the addition of EGCG at concentrations indicated. BrdU was added at the end of 24 hours incubation period for 2 hours, followed by BrdU assay. These figures are representative of three experimental replications, all with similar results. Data are expressed as percentage of untreated cells. Error bars indicate one standard deviation of the mean. Different capital letters indicate statistically significant differences among conditions (ANOVA, Tukey post-hoc test, α=0.05, n=4).

FIGS. 30A and 30B represent enzymatic activity and quantity determination of endogenous catalase and superoxide dismutase (SOD) in NHEK, OSC-2, and OSC-4 cells incubated with EGCG. FIG. 30A represents the enzymatic activities of catalase and total SOD were assayed after cells were incubated with 100 μM EGCG for 0, 6, 12, and 24 hours. Error bars indicate one standard deviation of the mean. Different capital letters indicate statistically significant differences among conditions (ANOVA, Tukey post-hoc test, α=0.05, n=4). FIG. 30B represents protein levels of catalase, Mn-SOD and actin were determined by Western blot in cells treated with 100 μM EGCG for 0, 6, 12, and 24 hours. The figure is a representative experiment repeated three times with similar results.

### DETAILED DESCRIPTION OF ILLUSTRATIVE EMBODIMENTS OF THE INVENTION

While a number of studies have shown that polyphenolic compounds, such as those found in green tea possesses chemopreventive and apoptotic activity against certain cancers, the pathways responsible for these activities have not been fully elucidated. The present invention demonstrates, for the first time, that a group of plant derived compounds can induce a cell cycle regulator in normal human cells in a time and dose dependent manner; demonstrating that p57 (KIP2), a CDK and apoptosis inhibitor, is an intracellular target for green tea polyphenols in normal human epithelial cells (keratinocytes), but not in the tumor cells. The present invention also demonstrates, for the first time, that a group of plant derived compounds are associated with the induction of caspase-14 in epidermal keratinocytes.

Also demonstrated by the present invention is an in vitro co-culture assay for anticancer drug screening based on the detection of tumor cell death and normal cell survival in a device in which normal cells are co-cultured with tumor cells. This assay may be used to identify potential agents that possess chemopreventive or therapeutic properties. This assay may also be used to test the potency and efficacy of potential or currently available agents that possess chemopreventive or therapeutic properties.

Naturally occurring phenolic compounds have been identified in green tea. These phenolic compounds are collectively referred to as green tea polyphenols, also referred to as “GTPPs.” At least four major constituent polyphenols have been identified within GTTP; (−)-epicatechin (also referred to as “EC”), (−)-epigallocatechin (also referred to as “EGC”), (−)-epicatechin-3-gallate (also referred to as “ECG”) and (−)-epigallocatechin-3-gallate (also referred to as “EGCG”). The most abundant green tea polyphenol, epigallocatechin-3-gallate (EGCG), has been tested to be able to access organs throughout the body (Suganuma et al., Mutat Res 1999;428:339-44).

As used herein, a polyphenolic composition contains one or more of the polyphenolic compounds of the type typically found in green tea. These polyphenolic compounds can be derived from green tea or can be synthetically produced. A polyphenolic composition may be, for example, a crude extract of green tea. A polyphenolic composition may be, for example, a mixture of green tea polyphenols (GTPPs). A polyphenolic composition may also be, for example, one or more of the purified polyphenolic constituents of GTTP, including, for example, one or more of EC, EGC, ECG, or EGCG.

Polyphenolic compositions are readily available. For example, a simple extract of green tea can be prepared by incubating a green tea bag for 10 minutes, followed by collection of the extract. GTPP and its four major polyphenolic constituents (EC, EGC, ECG, and EGCG) are commercially available. For example, a mixture of the four major GTTPs is commercially available from LKT Laboratories, Minneapolis, Minn. Likewise, purified EC, EGC, ECG, and EGCG are commercially available, for example, from Sigma-Aldrich, St. Louis, Mo.

For use in the methods of the present invention, a GTPP mixture or any of its four major polyphenolic constituents (EC, EGC, ECG, and EGCG) alone can be prepared in a wide range of concentrations. For example, a GTPP mixture or a preparation of one or more of its polyphenolic constituents can be prepared at concentrations similar to those found in green tea drink preparations. That is, about 300 μM to about 600 μM for EGCG (50 μM is 22.9 μg/ml) and about 0.38 mg/ml to about 0.76 mg/ml for GTTP. A GTPP mixture or a preparation of one or more of its polyphenolic constituents can be prepared at concentrations similar to physiological plasma concentrations. Physiological plasma concentrations of EGCG range up to about 4.4 μM.

Likewise, a preparation of a GTPP mixture or a preparation of one or more of its polyphenolic constituents can be prepared at concentrations greater than or lesser than physiological plasma concentrations. For example, EGCG can be prepared at concentrations of about 1 μM, about 2 μM, about 5 μM, about 10 μM, about 15 μM, about 50 μM, about 100 μM, about 200 μM, about 250 μM, or about 500 μM. GTTP can be prepared, for example, at concentrations of about 0.001 mg/ml, about 0.005 mg/ml, about 0.01 mg/ml, about 0.05 mg/ml, about 0.1 mg/ml, about 0.2 mg/ml, about 0.5 mg/ml, about 0.75 mg/ml, or about 1.0 mg/ml. The precise amount of a green tea polyphenolic compound, such as GTTP, EC, EGC, ECG, or ECGC, more preferably ECGC, used in any one embodiment of the present invention will vary according to factors known in the art including, but not limited to, the physical and chemical nature of the polyphenolic composition, the nature of the carrier, the intended dosing regimen, the state of the subject's immune system (e.g., suppressed, compromised, stimulated), the method of administering the polyphenolic composition, and the species to which the formulation is being administered. Accordingly, it is not practical to set forth generally the amount that constitutes an amount of a polyphenolic composition effective for all possible applications. Those of ordinary skill in the art, however, can readily determine the appropriate amount with due consideration based on the disclosure herein.

For use in the methods of the present invention, a polyphenolic composition may be formulated to include a “carrier.” As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances (i.e., one or more polyphenolic compounds) is well known in the art. Except insofar as any conventional media or agent is incompatible with the active ingredient, its use in the therapeutic compositions is contemplated. The phrase “pharmaceutically-acceptable” refers to molecular entities and compositions that do not produce an allergic or similar untoward reaction when administered to a human. The preparation of such compositions is well understood in the art.

In some embodiments, a polyphenolic composition, particularly one including a green tea polyphenolic constituent, such as EC, EGC, ECG, or EGCG, may be substantially pure. As used herein, “substantially pure” means sufficiently homogeneous to appear free of readily detectable impurities as determined by standard methods of analysis, such as thin layer chromatography (TLC), gel electrophoresis, high performance liquid chromatography (HPLC), used by those of skill in the art to assess such purity, or sufficiently pure such that further purification would not detectably alter the physical and chemical properties, such as enzymatic and biological activities, of the substance. Methods for purification of the compounds to produce substantially pure compounds are known to those of skill in the art. A substantially pure compound may, however, be a mixture of stereoisomers. In such instances, further purification might increase the specific activity of the compound.

The present invention shows, for the first time, that p57 induction by polyphenolic compositions in normal epithelial cells serves an anti-apoptotic function. Thus, the present invention includes methods of preventing damage to normal, non-cancerous cells in a subject undergoing cancer therapy by the administration of a polyphenolic composition under conditions effective to induce the expression of p57, induce the expression of caspase-14, or induce the expression of both p57 and caspase-14 in the non-cancerous cells. The polyphenolic composition may be administered to the subject prior to, coincident with, or subsequent to the cancer therapy. The cancer being treated can include a wide range of cancers, including, but not limited to, oral cancer, esophageal cancer, breast cancer, gastric cancer, colorectal cancer, prostate cancer, bladder cancer, skin cancer, and cervical cancer.

Also included in the present invention are methods of preventing damage to salivary glands cells, a condition also referred to as xerostomia, in subjects undergoing therapy for oral cancer or esophageal cancer. The method includes the administration to the subject of a polyphenolic composition under conditions effective to induce the expression of p57, induce the expression of caspase-14, or induce the expression of both p57 and caspase-14.

p57, also referred to herein as “KIP2” or “p57/KIP2,” is a potent, p53-independent, tight-binding G2 cyclin/CDK inhibitory protein (Lee et al., Genles Dev. 1995; 9:639-49). In vitro studies show induction of p57 leads to a potent growth arrest in G1 with concomitant hypophosphorylation of Rb and diminished E2 F-1 (Tsugu et al., Am J Pathol. 2000; 157:919-32).

Caspase 14, identified in 1998 from murine tissues (Ahmad et al., Cancer Res. 1998; 58:5201-5205; Hu et al., J Biol Chem. 1998; 273:29648-29653; Van de Craen et al., Cell Death Differ. 1998; 5:838-846), is expressed only in epithelial tissues, especially the epidermis. Unlike the other caspases, caspase 14 is not involved in the well-documented apoptotic caspase cascade, but is associated with terminal keratinocyte differentiation (Lippens et al., Cell Death Differ. 2000; 7:1218-1224; Eckhart et al., J Invest Dermatol. 2000; 115:1148-51; Pistritto et al., Cell Death Differ. 2002; 9:995-1006). Induction of caspase 14 at the transcriptional level is noted during stratum corneum formation (Eckhart et al., Biochem Biophys Res Commun. 2000; 277:655-659). Upon inhibition of cell differentiation, caspase 14 expression was diminished (Rendl et al., J Invest Dermatol. 2002; 119:1150-1155). Therefore, caspase 14 regulates epidermal differentiation, possibly by signaling terminal differentiation and cornification of the epidermis. In contrast, in pathological conditions such as psoriasis, in which cornification does not occur, the expression of caspase 14 is lacking (Lippens et al., Cell Death Differ. 2000; 7:1218-1224).

The induction of the expression of p57 or caspase 14 may be determined by any of many well know methods, including any of those described herein. Induction of the expression of p57 or caspase 14 may be determined by measuring the amount or activity of a desired gene product (for example, an RNA or a polypeptide encoded by the coding sequence of the gene). A biological sample can be analyzed. Preferably the biological sample is a bodily tissue or fluid, more preferably it is a bodily fluid such as blood, serum, plasma, urine, bone marrow, lymphatic fluid, and CNS or spinal fluid. In embodiments of the invention practiced in cell culture (such as methods for screening compounds to identify therapeutic agents), the biological sample can be whole or lysed cells from the cell culture or the cell supernatant.

Gene expression levels can be assayed qualitatively or quantitatively. The level of a gene product is measured or estimated in a sample either directly (for example, by determining or estimating absolute level of the gene product) or relatively (for example, by comparing the observed expression level to a gene expression level of another samples or set of samples). Measurements of gene expression levels may, but need not, include a normalization process.

Typically, mRNA levels (or cDNA prepared from such mRNA) are assayed to determine gene expression levels. Methods to detect gene expression levels include Northern blot analysis (see, for example, Harada et al., Cell 1990; 63:303-312), S1 nuclease mapping (see, for example, Fujita et al., Cell 1987; 49:357-367), polymerase chain reaction (PCR), reverse transcription in combination with the polymerase chain reaction (RT-PCR) (see, for example, Makino et al., Technique 1990; 2:295-301), and reverse transcription in combination with the ligase chain reaction (RT-LCR). Gene expression may be measured using an oligonucleotide microarray, such as a DNA microchip. DNA microchips contain oligonucleotide probes affixed to a solid substrate, and are useful for screening a large number of samples for gene expression.

Alternatively or in addition, polypeptide levels can be assayed. Immunological techniques that involve antibody binding, such as enzyme linked immunosorbent assay (ELISA) and radioimmunoassay (RIA), are typically employed. Where activity assays are available, the activity of a polypeptide of interest can be assayed directly.

With the present invention it has been demonstrated that the lack of a p57 stimulatory response in response to the administration of a polyphenolic composition results in the induction of caspase 3-dependent apoptosis. Caspase 3 plays an important role in apoptosis in human cancer cells (Chen et al., Arch Pharm Res, 2000; 236:605-12, Ahmad et al., J Natl Cancer Inst., 1997; 89:1881-6, Islam et al., Biochem Biophys Res Commun, 2000; 270:793-7, Hsu et al., General Dentistry, 2001; 50:140-146). Thus, the present invention includes methods of enhancing the effectiveness of a cancer therapy in a subject undergoing cancer therapy by the administration of a polyphenolic composition under conditions effective to induce caspase 3-dependent apoptosis in cancer cells. The present invention also includes methods of treating a precancerous oral lesion by contacting the precancerous oral lesion with a polyphenolic composition under conditions effective to induce p57 expression in normal epithelial cells and induce caspase 3-dependent apoptosis in precancerous and cancerous epithelial cells. Treatment of a precancerous oral lesion includes preventing the conversion of the precancerous cells of an oral lesion into cancerous cells, the preventing the conversion of normal cells into precancerous cells, the death of precancerous cells within the oral lesion and/or the death of cancerous cells within the oral lesion. Caspase 3-dependent apoptosis in precancerous and cancerous epithelial cells may be determined by any of many well know methods, including any of those described herein.

The present invention shows, for the first time, that polyphenolic compositions increase various cellular activities in epidermal keratinocytes, including the induction of caspase-14 and the down-regulation of p21/WAF1. Polyphenolic compositions are also associated with increased ATP production in aged keratinocytes, synthesis of new DNA synthesis in aged keratinocytes, and the promotion of differentiation in exponentially growing keratinocytes located in the basal layer of epidermis.

Thus, the present invention includes methods of treating a skin condition by contacting the skin with a polyphenolic composition under conditions effective to induce caspase-14 expression in keratinocytes. A wide variety of skin conditions may be treated, including, but not limited to, psoriasis, aphthous ulcer, actinic keratosis, rosacea, a wound, a burn, a skin condition associated with diabetes, a skin condition associated with aging, or a skin condition associated with altered keratinocyte differentiation. Treatment with a polyphenolic composition can also accelerate wound healing and regeneration of new skin tissue, subsequently preventing scar tissue formation. A polyphenolic may be administered topically for a sufficient period of time. Such a sufficient period of time may be, but is not limited to, at least one week, at least two weeks, at least three weeks, at least four weeks, at least five weeks, at least six weeks, at least eight weeks, at least one month, at least two months, at least three months, at least four months, at least six months, at least nine months, or at least twelve months. A polyphenolic composition may be administered as needed. For example, a polyphenolic composition may be administered weekly, two times a week, three times a week, four times a week, five times a week, six times a week, once a day, two times a day, three times a day, or more.

The polyphenolic compositions of the present invention may be administered by a wide variety of means, including, for example, orally, topically, parenterally, transdermally, and intranasally.

For oral administration, various delivery vehicles can be employed, including, but not limited to, aerosol carriers, mist and pump oral sprays, solutions, such as oral irrigators, mouth rinses and mouthwashes, or gels and solid compositions. Intra-oral sprays are well known to those familiar with the art of this industry. Such intra-oral sprays may be prepared in vials of variable sizes and milliliter concentrations that contain accordingly a predetermined number of metered sprays from non-aerosol pumps or with propellants for aerosol sprays. Dosages will depend on product compositions and labeled so that a predetermined number of sprays equals one daily dose. The preparations will be sprayed directly into the mouth at recommended intervals during the day. Various additives, carriers, diluents and adjuvants may also be utilized. Carriers that may be used include, for example, such solid delivery systems as oral gels, powders and toothpastes. The compositions of these are conventional and well known to those skilled in the manufacture of these products. Toothpaste base, for example, may include but is not limited to ingredients as calcium diphosphate, methyl cellulose, saccharin, glycerine, chlorophyll, sodium lauryl sulphate and others.

A polyphenolic composition may be incorporated into a vehicle for topical administration. Suitable topical application vehicles include, but are not limited to, creams, gels, foams, ointments, lotions, solutions, a suspension, dispersions, emulsions, microemulsions, pastes, powders, surfactant-containing cleaning preparations, solid sticks (e.g., wax- or petroleum-based sticks), wipes, oils, and sprays. Such a vehicle for topical administration may contain, for example, about 0.001%, about 0.002%, about 0.005%, about 0.01%, about 0.015%, about 0.02%, about 0.025%, about 0.05%, about 0.1%, about 0.25%, 0.5%, 0.75%, about 1%, about 2.5%, about 5%, about 7.5%, about 10%, about 25%, or about 50% of a polyphenolic composition. A suitable vehicle for topical administration may include additional active ingredients, for example, including, but not limited to, an antibiotic, a pain reliever, a skin penetration enhancer, or a topical anesthetic. In some embodiments, the polyphenolic composition may be incorporated into, for example, a sunscreen, a skin lotion, a skin moisturizer, or cosmetic. Alternatively, the polyphenolic composition may be incorporated into any vehicle suitable for intradermal or transdermal delivery.

For parenteral administration in an aqueous solution, the polyphenolic composition should be suitably buffered if necessary and the liquid diluent first rendered isotonic with sufficient saline or glucose. These particular aqueous solutions are especially suitable for intravenous, intramuscular, subcutaneous, intraperitoneal, and intratumoral administration. In this connection, sterile aqueous media that can be employed will be known to those of skill in the art in light of the present disclosure (see for example, “Remington's Pharmaceutical Sciences” 15 th Edition). Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by the FDA.

Therapeutically effective concentrations and amounts may be determined for each application herein empirically by testing the compounds in known in vitro and in vivo systems, such as those described herein; dosages for humans or other animals may then be extrapolated therefrom.

The active ingredient may be administered at once, or may be divided into a number of smaller doses to be administered at intervals of time. It is understood that the precise dosage and duration of treatment is a function of the condition being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions and methods.

In some embodiments, agents of the present invention may be administered to the subject in combination with other modes of treatment, including other modes of cancer therapy. Such other modes of cancer therapy include, but are not limited to radiation treatment, brachytherapy, external beam radiation, chemotheraphy, hormone therapy and antibody therapy. The administration of the agents of the present invention can take place before, during or after the other cancer therapy.

The efficacy of treatment may be assessed by various parameters well known in the art. This includes, but is not limited to, determinations of tumor size, location and vascularization, as determined by such methods including, but not limited to, X-rays, scans, magnetic resonance imaging, computerized tomography, various nuclear medicine techniques and algorithms to evaluate tumor size and burden in three dimensions. Angiography can be used to evaluate vascularization of tumors and other tissues.

The efficacy of the administration of a polyphenolic composition effective for the treatment of cancer may be demonstrated by such means, including, but not limited to, the inhibition of tumor growth, the inhibition of tumor progression, the inhibition of tumor spread, the inhibition of tumor invasiveness, the inhibition of tumor vascularization, the inhibition of tumor angiogenesis, or the inhibition of tumor metastasis.

The inhibition of tumor growth is a decrease in the growth rate of a tumor. It includes, but is not limited to, at least one of a decrease in tumor weight or tumor volume, a decrease in tumor doubling time, a decrease in the growth fraction or number of tumor cells that are replicating, a decrease in the rate in which tumor cells are shed, and/or a decrease in the ratio of cell production to cell loss within a tumor. The inhibition of tumor growth can also include the inhibition of tumor growth of primary lesions and/or any metastatic lesions.

For oral cancer, the inhibition of tumor progression includes the disruption or halting of the progression of premalignant lesions, also called leukoplakia, to malignant carcinoma.

The inhibition of tumor spread is the decrease in the dissemination of a tumor to other locations. This dissemination to other locations can be the result of the seeding of a body cavity or surface with cancerous cells from a tumor and/or the transport of tumor cells through the lymphatic system and/or circulatory system. The inhibition of tumor spread can also include the inhibition of tumor spread in primary lesions and/or any metastatic lesions.

The inhibition of tumor invasiveness is the decrease in the infiltration, invasion and/or destruction of the surrounding local tissues, including, but not limited to organs, blood vessels, lymphatics and/or body cavities. The inhibition of tumor invasiveness can also include the inhibition of tumor invasiveness in primary lesions and/or any metastatic lesions.

The inhibition of tumor vascularization is the decrease in the formation of blood vessels and lymphatic vessels within a tumor and to and from a tumor. The inhibition of tumor vascularization can also include the inhibition of tumor vascularization in primary lesions and/or any metastatic lesions.

The inhibition of tumor angiogenesis is a decrease in the formation of new capillaries and microvessels within a tumor. The inhibition of tumor angiogenesis can also include the inhibition of tumor angiogenesis in primary lesions and/or any metastatic lesions.

The inhibition of tumor metastasis is a decrease in the formation of tumor lesions that are discontinuous with the primary tumor. With metastasis tumor cells break loose from the primary lesion, enter blood vessels or lymphatics and produce a secondary growth at a distant site. In some cases the distribution of the metastases may be the result of the natural pathways of the drainage of the lymphatic and/or circulatory system. In other cases, the distribution of metastases may be the result of a tropism of the tumor to a specific tissue or organ. For example, prostate tumors may preferentially metastasis to the bone. The tumor cells of a metastatic lesion may in turn metastasis to additional locations. This may be referred to as a metastatic cascade. Tumor cells may metastasize to sites including, but not limited to, liver, bone, lung, lymph node, spleen, brain or other nervous tissue, bone marrow or an organ other than the original tissue of origin. The inhibition of tumor metastasis includes the inhibition of tumor metastasis in primary lesions and/or any metastatic lesions.

The present invention includes a method of determining if cancer cells are resistant to an agent, the method including determining the p57/KIP2 level in the cancer cells prior to contact with the agent; contacting the cancer cells with the agent; determining the p57/KIP2 level in the cancer cells after contact with the agent; and comparing the p57/KIP2 level in the cancer cells after contact with the agent to the p57/KIP2 level in the cancer cells prior to contact with the agent; wherein an increase in the p57/KIP2 level in the cancer cells after contact with the agent compared to the p57/KIP2 level in the cancer cells prior to contact with the agent indicates the cancer cells are resistant to the agent.

The present invention also includes a method of determining if cancer cells are sensitive to an agent, the method including determining the p57/KIP2 level in the cancer cells prior to contact with the agent; contacting the cancer cells with the agent; determining the p57/KIP2 level in the cancer cells after contact with the agent; and comparing the p57/KIP2 level in the cancer cells after contact with the agent to the p57/KIP2 level in the cancer cells prior to contact with the agent; wherein no increase in the p57/KIP2 level in the cancer cells after contact with the agent compared to the p57/KIP2 levels in the cancer cells prior to contact with the agent indicates the cancer cells are sensitive to the agent.

The present invention also includes a method of identifying an agent effective for the treatment of a cancer, the method including determining the p57/KIP2 level in cancer cells prior to contacting with the agent; contacting the cancer cells with the agent; determining the p57/KIP2 level in the cancer cells after contacting with the agent; and comparing the p57/KIP2 level in the cancer cells after contacting with the agent to the p57/KIP2 level in the cancer cells prior to contacting with the agent; wherein no increase in the p57/KIP2 level in the cancer cells after contacting with the agent compared to the p57/KIP2 level in the cancer cells prior to contacting with the agent indicates the agent is effective for the treatment of a cancer.

The present invention also includes a method of determining the therapeutic effectiveness of an agent, the method including contacting normal cells with the agent; determining the p57/KIP2 level in the normal cells after contacting with the agent; contacting cancer cells with the agent; determining the p57/KIP2 level in the cancer cells after contacting with the agent; and comparing the p57/KIP2 level in the normal cells after contacting with the agent to the p57/KIP2 level in the cancer cells after contacting with the agent; wherein a higher p57/KIP2 level in the normal cells compared to the p57/KIP2 level in the cancer cells indicates the agent is effective for the treatment of cancer. In this method, the normal cells and cancer cells may be co-cultured together.

And, the present invention also includes a method of optimizing the formulation of an agent for the treatment of a cancer, the method including contacting cancer cells with a first formulation of the agent; determining the p57/KIP2 level in the cancer cells contacted with the first formulation of the agent; contacting cancer cells with a second formulation of the agent; determining the p57/KIP2 level in the cancer cells contacted with the second formulation of the agent; and comparing the p57/KIP2 level in the cancer cells contacted with the first formulation of the agent to the p57/KIP2 level in the cancer cells contacted with the second formulation of the agent; wherein the formulation with the lower level of p57/KIP2 indicates the formulation of the agent more effective for the treatment of a cancer.

As has already been described herein, induction of the expression of p57/KIP2 may be determined by a wide variety of methods. For example, induction of the expression of p57/KIP2 may be determined by detecting the p57/KIP2 protein or by detecting the mRNA encoding the p57/KIP2 protein.

A wide variety of cancer cells, also referred to herein as “tumor cells,” may be used in the methods of the present invention. For example, cancer cells may be derived from a subject in need of, or already undergoing, cancer therapy. Tumor cells may be of human, primate or murine origin. Tumor cells may be derived from cell lines, such as, for example, an epithelial carcinoma cell line. The epithelial carcinoma cell line may be, for example, an oral squamous carcinoma cell line, a metastatic oral carcinoma cell line, or a breast epithelial carcinoma cell line.

Currently existing screening methods are insufficient for the identification of agents that possesses both a cytotoxic effect on tumor cells and a protective effect on normal, non-cancerous cells. The present invention provides an in vitro screening method that detects both survival of normal, non-cancerous cells and apoptosis of cancerous, tumor cells. This screening method is able to screen potential agents, including plant-derived agents, such as green tea polyphenolic compounds, based on the differential activation of the survival and apoptosis pathways. Tumor cell death and normal cell survival are detected simultaneously, in a device that co-cultures normal, non-cancerous human cells adjacent to human tumor cells. In some embodiments, the in vitro co-culture system utilizes double fluorescent detection of the activation of these two pathways. For example, using simple standard immuno-fluorescence microscopy techniques, the induction of apoptosis can be detected in tumor cells by the diminished green fluorescence of a transfected green fluorescent protein (GFP) and the induction of p57 expression in normal, non-cancerous cells can be concomitantly detected by increased red fluorescence.

The method involves co-culturing normal cells adjacent to tumor cells in vitro; contacting the co-cultured cells with an agent; determining if contact with the agent induces tumor cell death; and determining if normal cells survive upon contact with the agent; wherein the induction of tumor cell death by contact with the agent and the survival of normal cells upon contact with the agent indicated the agent possesses both a cytotoxic effect on tumor cells and a protective effect on normal cells.

A wide variety of both the tumor cells and normal cells may be used in the assay. For example, both the tumor cells and the normal, non-cancerous cells may be of the same histological origin. For example, both may be of epithelial origin. Both tumor cells and normal cells may be of human, primate or murine origin. Both tumor cells and normal cells may be derived from cell lines, such as, for example, an epithelial carcinoma cell line. The epithelial carcinoma cell line may be, for example, an oral squamous carcinoma cell line, a metastatic oral carcinoma cell line, or a breast epithelial carcinoma cell line.

The tumor cells may be a cell line stably transfected with GFP, obtained, for example, by the methods described herein. The tumor cell line stably transfected with GFP may be the human oral carcinoma cell line OSC-2 stably transfected with GFP. The normal, non-cancerous cells may be, for example, normal human primary epidermal keratinocytes or fibroblasts.

The induction of tumor cell death upon contact with an agent may be determined by a wide variety of methods, including any of the methods described herein. For example, tumor cell death may be determined by detecting apoptosis of the tumor cell. Apoptosis of the tumor cell line may be determined, for example, by detection of a green fluorescent protein (GFP).

The survival of normal cells upon contact with an agent may be determined by a wide variety of methods, including, for example, by any of the methods described herein. For example, survival of normal cells may be determined by detecting the induction of p57. As has already been described herein, induction of the expression of p57 may be determined by detecting the p57 protein or by detecting the mRNA encoding the p57 protein.

A unique feature of this system is the ability to detect tumor cell death and normal cell survival in a device in which normal human epithelial cells are co-cultured with human tumor cells. Although several in vitro co-culture systems using paired normal and malignant cells have been developed for anticancer drug screening (Appel et al., Cancer Chemother Pharmacol 1986; 17:47-52, El-Mir et al., Int J Exp Pathol 1998; 79:109-115, Torrance et al., Nat Biotechnol 2001; 19:940-945), these systems are not based on intracellular activation of specific pathways, and are not applicable to tissues such as human epidermal and mucosal tissues. The co-culture screening system of the present invention has many advantages. One, it more closely resembles the in vivo environment where normal cells and tumor cells are adjacent and interacting. Two, it reduces variation caused by separate culture of normal and tumor cells. Three, it facilitates elimination of a “false positive” agent, for example, one that kills both tumor and normal cells, which still is a major problem in conventional drug screening. And, four, it is able to detect differential pathways activated in normal versus tumor cells.

This method can be modified for high-throughput screening. For example, plant-derived compounds, numbering in the tens of thousands (King and Young, J Am Diet Assoc 1999; 99:213-8), could be efficiently screened for anticancer properties. Further, the principles of the system are adaptable to other pathways and cell lines.

The present invention also includes kits for the identification of an agent that possesses both a cytotoxic effect on tumor cells and a protective effect on normal cells. The kits include normal cells, tumor cells, and printed instructions, in a suitable packaging material in an amount sufficient for at least one assay. The tumor cells may be transfected with green fluorescent protein (GFP). The normal cells may be of the same histological origin as the tumor cells. The normal and tumor cells may cell lines. Additionally, the kit may include other reagents, such as buffers and solutions, needed to practice the invention.

As used herein, the phrase “packaging material” refers to one or more physical structures used to house the contents of the kit. The packaging material is constructed by well-known methods, preferably to provide a sterile, contaminant-free environment. The packaging material may have a label that indicates that the contents of the kit are to be used for the identification of an agent that possesses both a cytotoxic effect on tumor cells and a protective effect on normal cells. In addition, the kit contains printed instructions indicating how the materials within the kit are employed for the identification of an agent that possesses both a cytotoxic effect on tumor cells and a protective effect on normal cells. As used herein, the term “package” refers to a solid matrix or material such as glass, plastic, paper, foil, and the like, capable of holding within fixed limits a polypeptide. Thus, for example, a package can be a glass vial used to contain milligram quantities of a polypeptide. “Instructions for use” typically include a tangible expression describing the reagent concentration or at least one assay method parameter, such as the relative amounts of reagent and sample to be admixed, maintenance time periods for reagent/sample admixtures, temperature, buffer conditions, and the like.

The present invention further relates to agents that are identified according to the screening methods of the invention. Such agents can be used for the treatment of cancer, including, but not limited to oral cancer, esophageal cancer, gastric cancer, colorectal cancer, prostate cancer, bladder cancer, skin cancer, or cervical cancer. Such agents can also be used to promote wound healing and for the treatment of various skin conditions. Such skin conditions include, but are not limited to, psoriasis, rosceaca, diabetic skin conditions, the thinning of skin associated with aging, and skin conditions associated with altered keratinocyte differentiation. Such agents can be formulated for therapeutic use as described herein. Potential agents to be screened in the assays of the present invention may be derived from a wide variety of sources. For example, plant-derived compounds, numbering in the tens of thousands (King and Young, J Am Diet Assoc 1999; 99:213-8), could be efficiently screened. Candidate agents can also be identified by screening chemical libraries according to methods well known to the art of drug discovery and development (see Golub et al., U.S. Patent Application Publication No. 2003/0134300, published Jul. 17, 2003).

The methods of the present invention may be performed on any suitable subject. Suitable subjects include, but are not limited to, animals such as, but not limited to, humans, non-human primates, rodents, dogs, cats, horses, pigs, sheep, goats, or cows.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Chemopreventive Effects of Green Tea Polyphenols Correlate With Reversible Induction of p57 Expression

In this study, Western blot analysis combined with cycloheximide treatment was used to examine the effects of green tea polyphenols on expression levels of p57 (KIP2), a cyclin dependent kinase and apoptosis inhibitor, in normal human keratinocytes and the oral carcinoma cell lines SCC25 and OSC2. The results showed that the most potent green tea polyphenol, (−)-epigallocatechin-3-gallate (EGCG), induced p57 in normal keratinocytes in a dosage and time dependent manner, while levels of p57 protein in oral carcinoma cells were unaltered. The differential response in p57 induction was consistent with the apoptosis status detected by annexin V assay. This example indicates that the chemopreventive effects of green tea polyphenols involve p57 mediated cell cycle regulation in normal epithelial cells.

Materials And Methods

Chemicals and compounds. (−)-epigallocatechin-3-gallate (EGCG) was obtained from Sigma-Aldrich Corp. (St. Louis, Mo.). A mixture of GTPPs was purchased from LKT Lab. Inc. (Minneapolis, Minn.). The carcinogen NNK was purchased from Toronto Research Chemicals, Inc. (Toronto, Canada) and BaP was obtained from Sigma-Aldrich Corp., St. Louis, Mo. GTPPs and EGCG were dissolved in cell culture media and filter-sterilized immediately prior to use. The NNK and BaP were solubilized with DMSO. Annexin V-EGFP Apoptosis Kit was purchased from Clontech Lab. Inc., Palo Alto, Calif.

Cell lines and cell culture. The normal human keratinocytes (NHEK CC-2507) were obtained from Cambrex Bioscience (Baltimore, Md.). The SCC25 cell line (obtained from American Type Culture Collection, Manassas, Va.) was isolated from a squamous cell carcinoma of the tongue of a 70 year-old male (Rheinwald et al., Cell 1980; 22:629-32). The OSC2 cell line was isolated from a submandibular lymph node metastasis of a 68-year old female. The primary tumor was located in the gingiva of this patient (Osaki et al., Eur J Cancer B, Oral Oncol. 1994; 30B:296-301). OSC2 cells have a p53 mutation at exon 8, site 280, resulting in an Arg→Thr conversion (Yoneda et al., Eur J Cancer 1999; 35:278-83). SCC25 cells have undetectable p53 levels, while OSC2 cells over-express p53 (Huynh et al., Journal of Dental Research 2001; 80:176). SCC25 and OSC-2 cells were maintained in 45% Dulbecco's MEM medium (DMEM) or 45% Ham's F12 medium, supplemented with 10% newborn calf serum, 100 I.U./ml penicillin, 100 μg/ml streptomycin and 5 μg/ml hydrocortisone. The keratinocytes (two batches were used for repeatability) were cultured and maintained in KGM-2 medium (Cambrex). All cell cultures were maintained in a 37° C. incubator with 5% CO₂. For Western blot analysis, the keratinocytes were placed in KGM-D medium overnight prior to treatment. Rabbit anti-p57 and goat anti-actin antibodies used in this study were purchased from Santa Cruz Biotech Company (Santa Cruz, Calif.). Each experiment was repeated at least three times. Three batches of the normal human keratinocytes were tested with consistent results for p57 induction.

Cell treatments. For 24 hours treatments, exponentially growing cells with 40% confluency (to minimize differentiation and spontaneous apoptosis) were either maintained in 50 μM EGCG (2.29 mg/100 ml) or 0.2 mg/ml of GTPPs in tissue culture flasks (25 cm²). Control flasks contained cells without any treatment. To test whether BaP or NNK interfere with the induction of p57, the human keratinocytes were treated with 0.12 μM BaP, or 10 μM NNK, either alone or in combination with 50 μM EGCG. To examine the time course of p57 induction, cells were treated with 50 μM EGCG and harvested for Western analysis at 30 minutes, 2 hours, 6 hours, and 24 hours. The human keratinocytes were treated when the cell density reached 85-90% confluency (to mimic the epithelium).

In a parallel series of experiments, treatment with EGCG was performed with 30 micrograms per milliliter (μg/ml) cycloheximide added to the keratinocyte media 30 minutes prior to the addition of EGCG. The dose-response experiments were performed using EGCG concentrations at 30, 50 100, and 200 μM in the culture media for 24 hours.

Western blot analysis. Cells from different treatment groups were lysed in RIPA buffer (1% NP-40, 1% sodium deoxycholate, 0.1% SDS, 0.15 M NaCl, 0.01 M sodium phosphate, pH 7.2, and 1% Trasylol) containing proteinase inhibitors (1 mM PMSF, 1 μg/ml each of aprotinin, leupeptin, and pepstatin). The concentration of protein in each sample was determined using the BioRad DC Protein Assay and spectrophotometry. Fifty micrograms (μg) of protein from each sample and a BioRad molecular weight standard marker were run on a 10% SDS-PAGE, followed by transfer to nitrocellulose membranes. Nonspecific binding to membranes was blocked with 10% nonfat milk. Specific primary polyclonal (rabbit) antibody against p57 and a horse radish peroxidase-conjugated goat anti-rabbit secondary antibody were used in conjunction with the ECL Chemiluminescence Kit and membranes were exposed to radiographic films for detection.

Annexin V apoptosis assay. Initially, 10⁴ OSC2 cells were seeded in each chamber of an 8-chamber chamberslide and 5×10⁴ human keratinocytes were seeded in each well of a 24-well tissue culture plate. When the cells formed a monolayer in the center, fresh media containing 0.2 mg/ml of GTPPs was added, and 24 hours later, the Annexin V assay was performed according to the manufacturer's instructions with minor modifications. Visualization and photography were realized by confocol-fluorescence microscope imaging using a dual filter set for FITC and rhodamine.

Results And Discussion

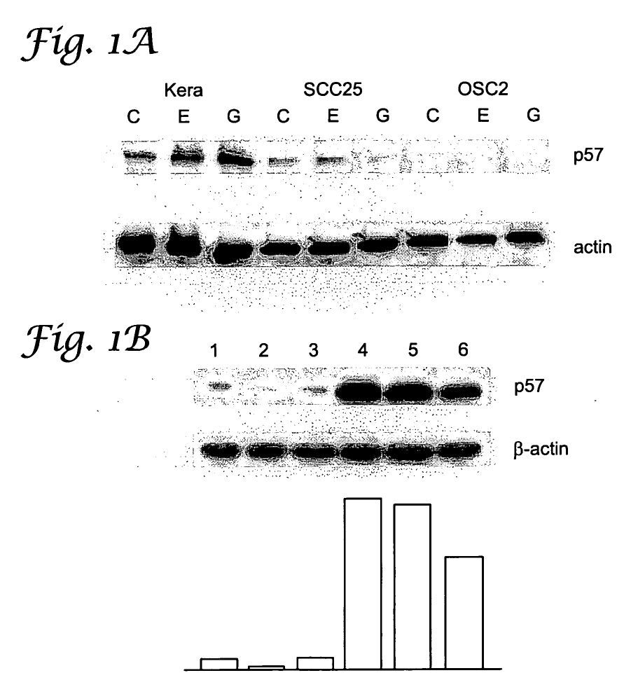

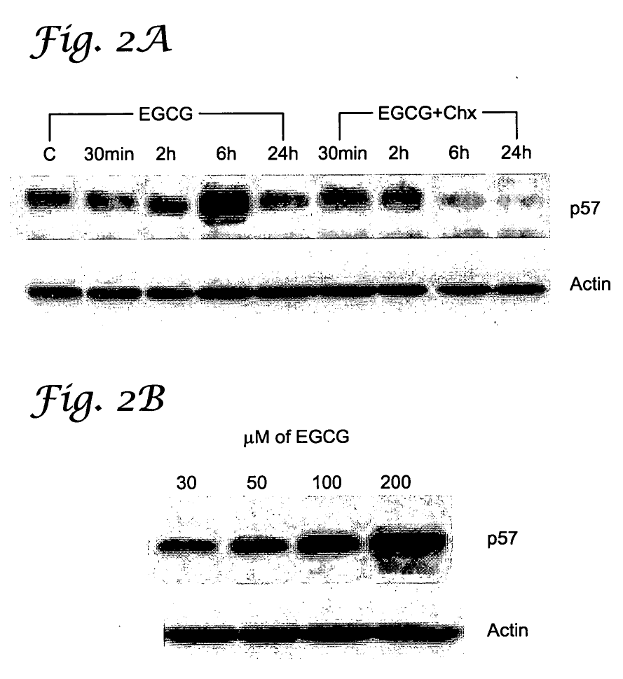

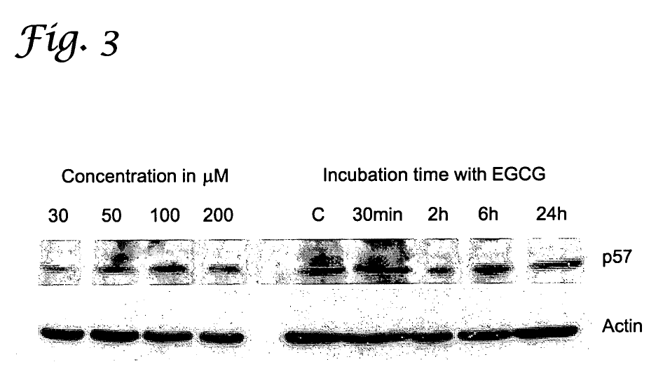

Data generated from this study showed a novel observation, p57 was accumulated in normal human epithelial cells, but not in oral carcinoma cells at 40% confluency, following incubation for 24 hours with either 50 μM EGCG or 0.2 mg/ml GTPPs (FIG. 1A). The keratinocytes consistently showed modestly higher levels of actin (used as a loading control) than the oral carcinoma cells when the same amount of the protein was loaded. When BaP and NNK were present, EGCG-stimulated p57 protein accumulation still occurred in the epithelial cells, although at slightly lower levels compared to EGCG alone (FIG. 1B). BaP and NNK alone did not alter p57 expression, as shown in lanes 2 and 3 of FIG. 1B. When the normal epithelial cell density was 90%, p57 expression induced by 50 μM EGCG reached its peak at 6 hours, and declined to basal levels by 24 hours (FIG. 2A). This contrasts with the results obtained at 40% confluency (FIG. 1A) where p57 levels remained high. However, when the EGCG concentration was increased to 100 μM or 200 μM, p57 levels in cells at 90% confluency remained high throughout the 24 hours period examined (FIG. 2B). Thus, the induction of p57 by EGCG is dependent on the dose, time and confluency of the cells. Treatment with cycloheximide resulted in a gradual decline in p57 protein levels, while actin levels remained relatively constant, indicating that in normal human keratinocytes, the p57 protein accumulation induced by 50 μM EGCG was the result of new synthesis instead of decreased degradation (FIG. 2A, EGCG+Chx). The metastatic oral carcinoma OSC2 cells failed to elevate p57 expression in response to EGCG at any time point or concentration (FIG. 3). The annexin V apoptosis detection assay demonstrated that 0.2 mg/ml GTPPs induced differential response in apoptotic status from the normal epithelial cells and the oral carcinoma cells.

As indicated by annexin V-FITC, which binds to apoptotic cells that expose phosphatidylserine molecules on the outer layer of the cell membrane, OSC2 cells treated with GTPPs for 24 hours showed massive apoptosis, compared with untreated cells. In contrast, the keratinocytes did not exhibit any phosphatidylserine translocation in the control nor in GTPPs treated samples.

It is a significant observation that a group of plant derived compounds can induce a cell cycle regulator in normal human cells in a time and dose dependent manner. Therefore this example reports, for the first time, that p57 (KIP2), a CDK and apoptosis inhibitor, is an intracellular/nuclear target for green tea polyphenols in normal human epithelial cells (keratinocytes), but not in the tumor cells tested. Furthermore, the tobacco carcinogens BaP and NNK do not markedly inhibit this p57 induction (FIG. 1B). p57 induction in the normal epithelial cells showed remarkable correlation with apoptosis resistance. This correlation of p57 induction (FIG. 1A) and resistance to GTPPs-induced apoptosis only in normal human keratinocytes suggests that p57 may be involved in mechanisms that enhance cell survivability during GTPPs treatment. The N-terminus of p57 protein is able to bind CDK-cyclin complex and inhibit its kinase activity; the C-terminus of p57 protein contains a proliferating cell nuclear antigen (PCNA)-binding domain that suppresses cell proliferation (Watanabe et al., Proc Natl Acad Sci 1998; 95:1392-7). It is possible that elevated p57 protein expression may induce reversible growth arrest in the normal keratinocytes and prevent E2F mediated apoptosis through hypophosphorylation of Rb protein (Tsugu et al., Am J Pathol. 2000; 157:919-32). In contrast, oral carcinoma cell lines derived from a primary site and from a metastasis failed to elevate p57 expression, and are unable to survive the apoptotic effect of GTPPs/EGCG. This differential response in p57 induction explains why green tea polyphenol-mediated apoptosis has been found only in tumor cells (Paschka et al., Cancer Lett. 1998; 130:1-7, Chen et al., Cancer Lett. 1998; 129:173-9, Islam et al., Biochem Biophys Res Commun. 2000; 270:793-7, Yang et al., Carcinogenesis. 1998; 19:611-6, Paschka et al., Cancer Lett. 1998; 130:1-7). In addition, EGCG concentration and cell density are two determinant factors for the duration of each p57-induction cycle

The example demonstrates that a group of plant-derived compounds, green tea polyphenols, are able to induce p57 protein expression in human keratinocytes but not in two oral cancer cell lines tested. This induction is dosage- and time-dependent. It is apparent that exposure to GTPPs/EGCG reversibly increases the level of intracellular p57 expression in the normal human epithelial cells tested, which may serve as a dual-effect chemopreventive mechanism. When cells lose the p57 response as in tumor cells, these cells would be defenseless and selectively induced to apoptosis by GTPPs/EGCG. Therefore, frequent consumption of green tea or green tea polyphenols may contribute to chemoprevention against oral cancer.

### Example 2

### Chemoprevention of Oral Cancer by Green Tea

In this example the effect of GTP/EGCG on normal keratinocytes and oral squamous cell carcinoma (SCC) cells was investigated in order to elucidate molecular parameters that might be protective for normal cells and cause cell death in neoplastic cells. These observations would provide a scientific basis for green tea as a bona fide chemopreventive agent for oral malignancy and justify its institution as a safe public health strategy.

Cancer is increasingly viewed as a cell cycle disease. In eukaryotes, the cell cycle is controlled by a number of cell cycle regulators such as cyclin dependent kinases (CDKs) and CDK inhibitors (CKIs). CKIs regulate the cell cycle by imposing growth arrest. When growth arrest occurs in normal human keratinocytes, these cells became resistant to apoptosis signals such as UV light. Published reports have not shown significant induction of cell cycle regulator proteins by GTP/EGCG, which may be ascribed to limited data available in normal human epithelial systems. A novel observation was made when certain CKIs were profiled in response to GTP/EGCG in both normal epithelial cells and tumor cells, the CKI p57 (KIP2) is specifically induced by GTP/EGCG only in normal human epithelial cells (as shown in Example 1). The significance of this finding is that a group of plant-derived compounds are able to specifically induce a human gene product in a dose and time-dependent manner for either cell survival or apoptosis.

Based on these observations, green tea polyphenols should induce p57 in normal epithelial cells, serving an anti-apoptosis function; in tumor cells, failure to elevate p57 levels in the presence of the polyphenols may result in induction of caspase 3 (the key limiting enzyme for apoptosis) dependent apoptosis.

Materials And Methods

Chemicals and compounds. A mixture of GTP was purchased from LKT Laboratory, Inc. (Minneapolis, Minn.). Annexin V-FITC Apoptosis Kit was purchased from CLONTECH Laboratory, Inc., Palo Alto, California. Crude green tea extract was prepared by incubation of 4 ml cell culture media with a green tea bag (P.R.I., New Jersey) for 10 minutes followed by collection of the extract.

Cell lines and cell culture. The normal human keratinocytes (NHEK CC-2507) were obtained from Cambrex (Baltimore, Md.). The SCC25 cell line (obtained from American Type Culture Collection, Manassas, Va.) was originally isolated from a squamous cell carcinoma of the tongue of a 70 year-old male (Rheinwald and Beckett, Cell, 1980; 22:629-32). The OSC2 cell line was isolated from a submandibular lymph node metastasis of a 68-year old female. The primary tumor was located in the gingiva of this patient (Osaki et al., Eur J Cancer B, Oral Oncol. 1994; 30B:296-301). SCC25 cells have undetectable p53 levels, while OSC2 cells over-express p53 (Huynh et al., J Dental Research, 2001; 80:176). The DOK cell line is a dysplastic immortal oral keratinocytes cell line (Chang et al., Int J Cancer 1992; 52:896-902). SCC25, DOK and OSC2 cells were maintained in 45% Dulbecco's MEM medium (DMEM), 45% Ham's F12 medium and 10% newborn calf serum, 100 I.U/ml penicillin, 100 μg/ml streptomycin and 5 μg/ml hydrocortisone. The keratinocytes were cultured and maintained in KGM-2 medium (Cambrex). All cell cultures were maintained in a 37° C. incubator with 5% CO₂.

Annexin V apoptosis assay. Initially, 10⁴ of tumor cells were seeded in each chamber of an 8-chamber chamberslide and 5×10⁴ human keratinocytes were seeded in each well of a 24-well tissue culture plate, and the monolayers were subjected to 24 hour-0.2 mg/ml of GTP treatment, followed by the Annexin V assay according to the manufacturer's instructions with minor modifications.

Cell growth assay. Cells (2×10⁵) were seeded in each T25 culture flask with 5 ml DMEM/F12 medium for 48 hours. The treatments were started with 50 μM EGCG for 24 hours, 48 hours and 96 hours. At each time point, the cell numbers were counted using a hemacytometer with the presence of Trypan blue.

Cell invasion/migration assay. The invasion/migration assays were conducted using a Transwell apparatus (Costar) with 6.5 mm diameter wells and membranes of 8 μm pore size. The invasiveness at each time point was tested in DMEM/F12 medium immediately following the cell growth assay, by seeding 10⁵ cells in each transwell. Cells migrated across the transwell membrane were counted as per microscopic field.

Results and Discussion

Morphological change was observed when OSC2 cells were exposed to green tea crude extract (25 μl/ml) for 1 hour during a 12 hour period in comparison with untreated cells. When these cells were exposed to green tea crude extract for a second 1 hour incubation within a 24 hour period, apoptotic cells were apparent with reduced size, loss of contact and uncharacterized nuclei. When green tea crude extract (16 μl/ml) was incubated un-interruptedly with oral carcinoma cell line OSC2 for 6 hour, many cells were disfigured and detached, by 24 hour, massive cells death was observed and increased debris from cell lysis in comparison to the untreated cells. Magnifications used included 400×and 100×.

Thus, it is evident that green tea is a powerful inducer of apoptosis in tumor cells. One hour incubation of a small percentage of green tea crude extract (80 μl/5 ml) was able to induce morphological change in OSC2 cells comparing to untreated controls. Two one-hour incubations of the crude extract separately within a 24-hour period further increased the number of dead cells. When green tea crude extract at 125 μl/5 μl was continuously incubated with OSC2 cells for 6 hours or 24 hours, the majority of the cells underwent cell death comparing to the control and cells incubated with green tea crude extract for 24 hours were not able to recover when they were placed back to normal media. This result suggested that exposure to green tea could lead to elimination of oral cancer (squamous cell carcinoma) cells.

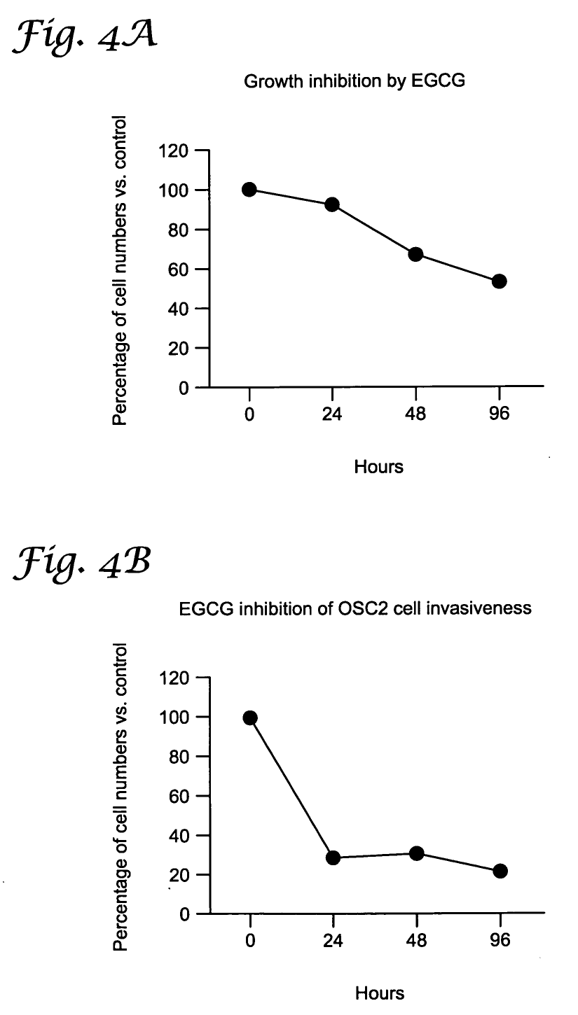

Based on this observation, 0.2 mg/ml GTP was applied on a oral cancer progression model system that consists of normal human epithelial cells (pooled newborn epidermal keratinocytes), a pre-cancerous dysplastic oral keratinocyte cell line DOK, a primary oral carcinoma line SCC25, and a metastatic oral carcinoma line OSC2. To examine the status of apoptosis, 0.2 μg/ml GTP was incubated with exponentially growing cells for 24 hours followed by Annexin V apoptosis assay. As indicated by the presence of annexin V-FITC (green), OSC2 and SCC25 cells treated with GTP for 24 hours showed massive apoptosis, compared with untreated cells. In contrast, normal epithelial cells did not exhibit any apoptotic cells in the control (FIG. 4A) nor in GTP treated samples (FIG. 4B), and there was no massive apoptosis in DOK cells. This result indicated that GTP differentially induced apoptosis in oral cancer cells and the apoptosis pathway was not p53 dependent, since OSC2 cells have a mutated and overly expressed p53, and SCC25 cells do not express p53.

To further investigate this property of GTP, the most potent component, EGCG, was used at a lower concentration (50 μM, which is 1/7 weight/weight (w/w) of that of 0.2 mg/ml GTP) to determine its impact on OSC2 cells. EGCG was effective in inhibiting cell growth within 24 hours. By day four, the number of EGCG-treated OSC2 cells was only 50% compared to the controls. Inhibition of cell invasiveness/migration was rapid. After 24 hours of treatment, cells invading the membrane were reduced to about 30% of control. Following 96 hours of treatment, the percentage was further reduced to 20%. These data suggest that EGCG is able to both reduce the mobility of metastatic oral cancer cells and inhibit their growth. Based on the observations that GTP/EGCG induced a differential response between normal and oral cancer cells, potential intracellular targets of GTP/EGCG were searched for and it was observed that a cyclin dependent kinase inhibitor p57 was significantly altered only in the normal cells in response to GTP/EGCG. To determine whether p57 is an intracellular target for GTP/EGCG, Western analysis was performed using the normal human keratinocytes and two oral carcinoma cells lines SCC25 and OSC2 in a separate study (see Example 1). The result showed that EGCG specifically induced p57 in normal keratinocytes, while levels in oral carcinoma cells were unaltered. Treatment of normal human keratinocytes at 40% confluency with 50 μM of EGCG induced up to a 12-fold increase of p57 expression, and the induction of p57 expression is time- and dose-dependent. In contrast, OSC2 cells (and several squamous cell carcinoma lines examined) failed to elevate p57 expression in response to EGCG at any time point or concentration. Therefore, p57 could serve as a target for GTP/EGCG in the normal epithelial cells to initiate a survival mechanism while oral cancer cells lacking the p57 response would undergo the apoptosis pathway.

Taken together, when normal epithelial cells (with the p57 response) are exposed to green tea/GTP/EGCG, induction of p57 (accompanied with other possible events) would enable the cells to survive, possibly through growth arrest or differentiation. On the other hand, oral carcinoma cells (without the p57 response) would undergo a specific apoptosis pathway. This example indicates that lack of a p57 response to EGCG leads to mitochondria-mediated, caspase 3 dependent apoptosis.

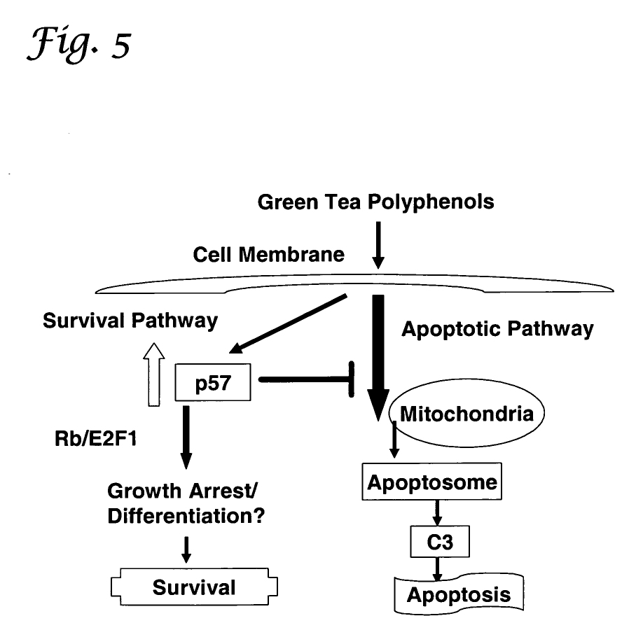

The data from this example indicate green tea and/or its constituents (EGCG) combat oral malignancy, including precancer and oral cancer. The data indicate that green tea polyphenols activate two pathways; one, survival through p57 induction, and, two, caspase 3-dependent apoptosis without p57 induction. The data also indicate that p57 induction by green tea polyphenols in normal epithelial cells serves as an anti-apoptotic function. Lack of the p57 stimulatory response to the presence of the polyphenols results in induction of caspase 3-dependent apoptosis (FIG. 5). In conclusion, the nature of the chemopreventive effects of green tea is believed to rest, in part, on its ability to signal a given cell and trigger a specific gene/cellular response, which directs the cell to undergo either survival or apoptosis pathway.

### Example 3

### Induction of p57 Is Required for Cell Survival When Exposed to Green Tea Polyphenols

In this Example, the correlation between p57 expression and survival/apoptosis was investigated by Western blot analysis, caspase 3 assays and morphological analysis. It is demonstrated that in the cells that lack p57 induction, green tea polyphenols induced Apaf- 1 expression along with caspase 3 activation, leading to apoptosis. In contrast, cells with polyphenol-inducible p57 maintained constant levels of Apaf-1 and proliferating cell nuclear antigen (PCNA), with basal caspase 3 activity. Retroviral-transfected, p57-expressing oral carcinoma cells showed significant resistance to green tea polyphenol-induced apoptosis. These results suggest that p57/KIP2 is a determinant pro-survival factor for cell protection from green tea polyphenol-induced apoptosis.

Example 1 demonstrated that p57/KIP2 induction is associated with cell survival of epidermal keratinocytes exposed to green tea polyphenols at concentrations that otherwise would cause apoptosis in tumor cells. The p57 gene product is a potent, p53 independent, tight-binding G1 cyclin/CDK inhibitory protein (Lee et al., Genes Dev. 1995; 9:639-49). The C-terminus of p57 protein possesses a binding domain for PCNA (Watanabe et al., Proc Natl Acad Sci USA 1998; 95:1392-7). Embryonic development in mice requires p57 expression; absence of it resulted in early postnatal death and growth retardation (Takahashi et al., J Biochem (Tokyo) 2000; 127:73-83, Yan et al., Genes Dev 1997; 11:973-83). On the other hand, in human intestinal cell models, elevation of p57 expression was associated with intestinal cell differentiation (Deschenes et al., Gastroenterology. 2001; 120:423-438). T-lymphocytes protect themselves from apoptosis by maintaining high levels of p57 (Vattemi et al., J Neuroimrniuol, 2000; 111:146-51). Recent pathological studies demonstrated that tumor specimens express lower levels of p57 protein compared to paired normal tissues, and low levels of p57 often correlate with poor prognosis (Ito et al., Liver 2000; 22:145-149, Ito et al., Oncology 2001; 61:221-5, Ito et al., Pancreas 2001; 23:246-50, Ito et al., Int J Mol Med 2002; 9:373-6). In vitro studies using human astrocytoma cells showed that induction of p57 led to growth arrest in G1, with concomitant hypophosphorylation of Rb and diminished E2 F-1 (Tsugu et al., Am J Pathol, 2000; 157:919-32). Therefore, it appears that p57 plays an important role in inhibition of apoptosis, since at least two apoptotic pathways can be activated by E2F independent of p53, through activation of p73 (Irwin et al., Nature, 2000; 407:645-8, Lissy et al., Nature 2000; 407:642-5, Yoneda et al., Eur J Cancer, 1999; 35:278-83) or apoptotic protease activating factor-1 (Apaf-1) (Moroni et al., Nat Cell Biol, 2001; 3:552-8). Both pathways require cytochrome c release from the mitochondria and apoptosome formation, which consists of cytochrome c, procaspase 9 and oligomerized Apaf-1 (Zou et al., J Biol Chem, 1999; 274:11549-56). Apaf-1 was first identified in 1997 (Zou et al., Cell, 1997; 90:405-13) and proved to be a limiting key factor for mitochondrion-mediated apoptosis (Cecconi, Cell Death Differ, 1999; 11: 1087-98). Binding with cytochrome c activates Apaf-1; it hydrolyses ATP or dATP to oligomerize into a large complex. This complex then binds and activates procaspase 9 and subsequently initiates the caspase pathway towards apoptosis (Zou et al., J Biol Chem, 1999; 274:11549-56). Cells without Apaf-1, such as certain malignant melanomas, are resistant to chemotherapy (Soengas et al., Nature, 2001; 409:207-11).

Example 2 demonstrated that p57 induction by green tea polyphenols, especially epigallocatechin-3-gallate (EGCG), leads to a cell survival pathway. In order to determine whether cells lacking p57 response would fail to survive the EGCG challenge, two normal human cell types were compared, a mammary epithelial cell population from one individual without the p57 response to EGCG treatment, and pooled epidermal keratinocytes that respond to EGCG by p57 induction. Furthermore, retroviral-transfected, p57-expressing metastatic oral squamous cell carcinoma OSC2 subclones also were examined to evaluate the impact of p57 expression in response to green tea polyphenol-induced apoptosis.

Materials and Methods

Chemicals and antibodies. EGCG was purchased from Sigma (St. Louis, Mo.). A mixture of four major GTPPs was purchased from LKT Lab. Inc (Minneapolis, Minn.). GTPPs and EGCG were dissolved in cell culture medium and filter-sterilized immediately prior to use. Rabbit anti-human p57, Apaf-1, PCNA and goat anti-human Actin antibodies used in this study were purchased from Santa Cruz Biotech Company (Santa Cruz, Calif.).

Cell lines and cell culture. The normal human keratinocytes (NHEK CC-2507) were purchased from Cambrex (East Rutherford, N.J.) and maintained in KGM-2 medium (Cambrex). The OSC2 cell line was previously described in Example 1. The OSC2 subclones were established by retroviral transfection of the parental OSC2 cell line. These clones were maintained in 45% Dulbecco's Modified Eagle's Medium (DMEM), 45% Ham's F12 medium and 10% fetal calf serum, 100 I.U/ml penicillin, 100 μg/ml streptomycin and 5 μg/ml hydrocortisone. The normal human mammary epithelial cells (HMEC) were maintained in MEGM medium (Cambrex). All cell cultures were maintained in a 37° C. incubator with 5% CO₂. Light photographs were taken with a SPOT RT digital camera system (Diagnostic Instruments, Sterling Heights, Mich.) linked to a Nikon Phase Contrast-2 microscope at an original magnification of 400×. Fluorescent photomicrographs were taken with a SPOT color digital camera system using the ZEISS Axiovert 10. Fluorescence was generated by a ZEISS AttoArc 2 source with an original magnification of 400×. Experiments were repeated three times.

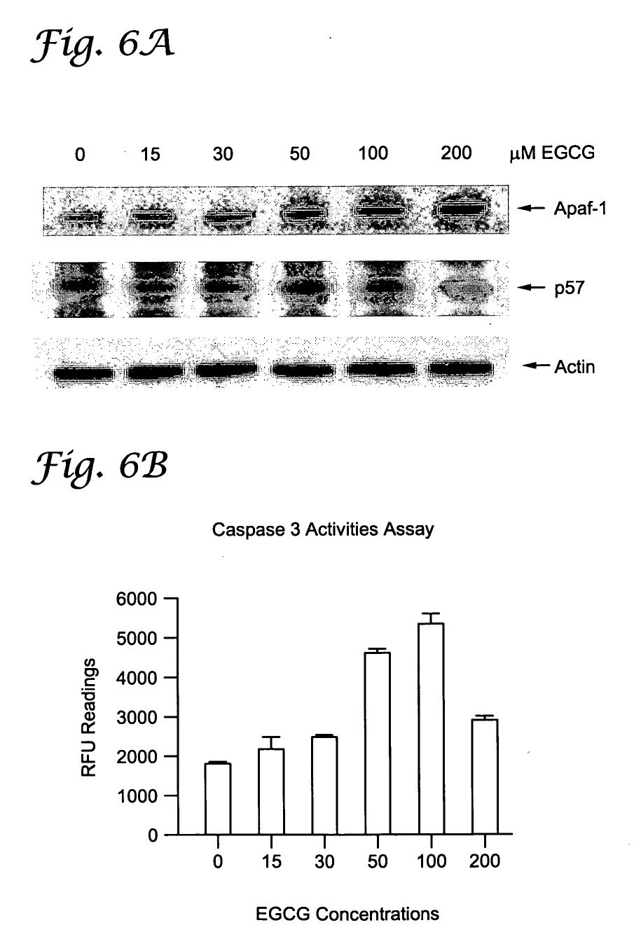

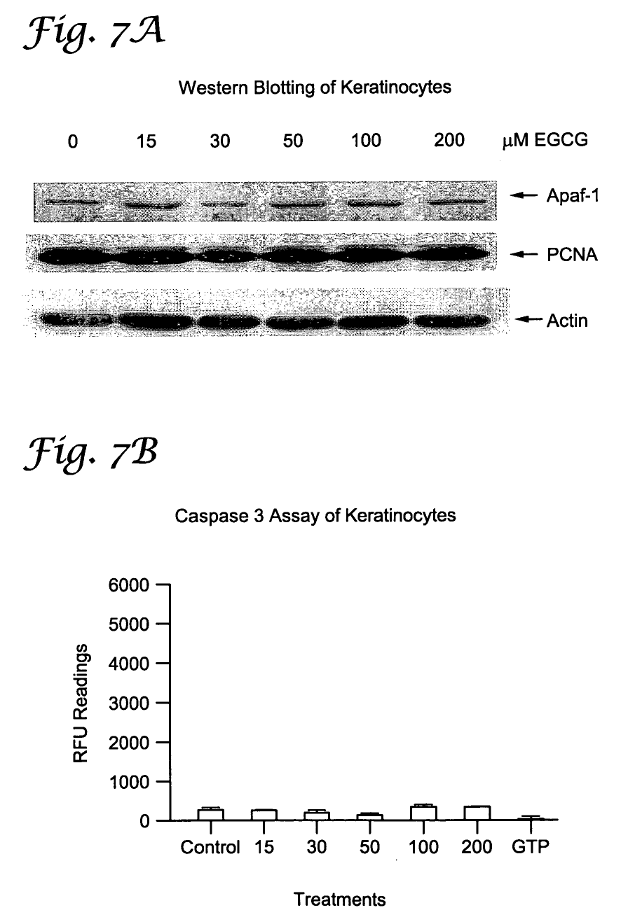

Western blot analysis. The keratinocytes and the mammary epithelial cells were placed in KGM-2 and MEGM, respectively, overnight prior to treatment. Cells were lysed, after 24-hour treatment, in RIPA buffer (1% NP-40, 1% sodium deoxycholate, 0.1% SDS, 0.15 M NaCl, 0.01 M sodium phosphate, pH 7.2, and 1% Trasylol) containing proteinase inhibitors (1 mM PMSF, 1 μg/ml each of aprotinin, leupeptin, and pepstatin). The concentration of protein in each sample was determined using the BioRad DC Protein Assay and spectrophotometry. 50 μg of protein from each sample and a BioRad molecular weight standard marker were run on a 10% SDS-PAGE, followed by transfer to nitrocellulose membranes. Nonspecific binding to the membranes was blocked with 10% nonfat milk. Primary polyclonal (rabbit) antibodies and a horseradish peroxidase-conjugated goat anti-rabbit secondary antibody were used in conjunction with the ECL Chemiluminescence Kit (Amersham Pharmacia Biotech, New Jersey) and membranes were exposed to radiographic films for detection. Western blots were digitized for comparison of the intensity for each band using the ImageTool image analysis software program (University of Texas Health Science Center, San Antonio, Tex.). The integrated density of each band was measured using identical 1480 pixel areas of each Apfa-1 or Actin band at a scale of densities from 0 to 255. The ratios of the integrated densities for Apfa-1/Actin are compared for mammary epithelial cells in FIG. 6A and keratinocytes in FIG. 7A.

Caspase 3 activity assay. The Caspase 3 Apoptosis Detection Kit was purchased from Santa Cruz Biotech. Inc. In a 24 well tissue culture plate, 10⁵ cells/well of control or treated cells in triplicates were plated. After 24 hour treatments with EGCG and GTPPs, the cells in each well were washed with 1 ml PBS and incubated with 100 μl lysis buffer on ice for 10 minutes. To each well, 100 μl of 2×reaction buffer was added with 10 mM DTT. Finally, 5 μl of DEVD-AFC substrate was added to each well containing cell lysates. The reaction mixtures were incubated for 1 hour at 37° C. The caspase 3 activity in each well was measured using a fluorescence plate reader set for 405 nanometer (nm) excitation and 505 nm emission.

Results and Discussion

The mammary epithelial cells maintained basal levels of p57 protein regardless of EGCG exposure (FIG. 6A). In contrast, protein levels of Apaf-1 were increased in conjunction with increased EGCG concentrations. Densitometry measurement demonstrated that the Apaf-1 protein levels were increased from 47% to 260% above control when EGCG concentration increased from 15 to 200 μM, while no significant changes were found in p57 levels (FIG. 6A). The epidermal keratinocytes have been previously characterized for their response to EGCG or GTPPs resulting in p57 induction without apoptosis (see Examples 1 and 2). In response to increasing concentrations of EGCG, these cells expressed stable basal levels of Apaf-1 and consistent high levels of PCNA (FIG. 7A).

The mammary epithelial cells responded to EGCG by a linear elevation of caspase 3 activities with the exception of 200 μM EGCG (FIG. 6B). The keratinocytes, however, only exhibited basal levels of caspase 3 activities (FIG. 7B). The mammary epithelial cells showed little change in morphology 24 hours after incubation with 50 μM EGCG, in comparison to the control cells. Significant cell death was observed after 48-hour treatment with 50 μM EGCG compared to 48 hour control cells. Morphological changes were seen as alterations in cell shape as well as cell blebbing. In addition, many cells appeared to be flattened, and the occupied space was still less than that observed in the untreated controls. At 96 hours, these characteristics were more apparent compared to the control, which became a confluent monolayer.

In 0.2 mg/ml GTPPs for 48 hours, both p57-transfected OSC2 clones demonstrated significant resistance to GTPPs-induced apoptosis. The trypan blue staining was noted only in the superficial stratum of cells, with a large number of living cells attached. The p57 antisense-transfected clones did not survive the GTPPs exposure. In fact, all cells were lysed or stained with trypan blue. The green fluorescent protein (GFP)-transfected clone, as an internal control, showed identical apoptosis to the parental cells (see Example 1) by cell lysis with diminished green fluorescence, while the untreated controls exhibited bright green fluorescence.

Unlike the keratinocytes, mammary epithelial cells under in vivo conditions could not be exposed to EGCG concentrations higher than 4.4 μM, the maximum human plasma concentration (Miyazawa, Biofactors, 2000; 13:55-59). Concentrations higher than that are potentially damaging to mammary epithelial cells, as shown in this example. The fundamental difference in response to EGCG between mammary epithelial cells and the epidermal keratinocytes is that p57 induction-associated cell survival is only present in the keratinocytes. In the mammary epithelial cells, while p57 protein levels remained unchanged, Apaf-1 levels increased as high as 260% in response to increasing concentrations of EGCG. In addition, increased caspase 3 activities paralleled increased EGCG concentration; 100 μM EGCG induced a 3-fold increase in caspase 3 activity at 24 hours compared to control. Lowered caspase 3 activity in 200 μM EGCG is possibly due to a plateau of the caspase 3 activity. The mammary epithelial cells have a higher background in caspase 3 activity than the keratinocytes, possibly due to a larger cell population undergoing apoptosis constitutively in mammary epithelial cells compared to the keratinocytes.

Apaf-1 accumulation, caspase 3-activation, and cell detachment/shrinkage and blebbing often are observed in mitochondrion-mediated apoptosis, and these characteristics are exhibited by EGCG-treated mammary epithelial cells. These results indicate that absence of p57 response to EGCG may lead to mitochondrion-mediated apoptosis even in normal cells. In addition, these results also suggest that EGCG concentrations higher than the maximum plasma concentration may be applied only topically (including oral application). In contrast, the normal epidermal keratinocytes showed constant basal levels of Apaf-1 regardless of time or dose of EGCG treatment, indicating a mechanism resisting apoptosis. During development, p57 and Apaf-1 may work collaboratively since the only cyclin dependent kinase inhibitor essential to development is p57 (Nishimori et al., J Biol Chem, 2001; 276:10700-5), and Apaf-1 also is actively involved (Moroni et al., Nat Cell Biol, 2001; 3:552-8). The survival/death linkage between p57 and Apaf-1 through the Rb/E2F pathway also may play an important role in regulation of differentiation and apoptosis in epidermal epithelial cells.

Based on the evidence that normal human keratinocytes in growth arrest are resistant to apoptosis (Chaturvedi et al., J Biol Chem, 1999; 274:23358-67), GTPPs/EGCG failed to induce apoptosis in the keratinocytes (Examples 1 and 2), and p57 induces G1 growth arrest and differentiation (Deschenes et al., Gastroenterology 2001; 120:423-438, Tsugu et al., Am J Pathol, 2000; 157:919-32), it is evident that p57-induction protects the human epithelium from green tea-induced apoptosis, possibly through growth arrest and/or differentiation, and cells failing to elevate p57 would enter a mitochondrion-mediated, caspase 3-dependent apoptotic pathway.

The role of p57 in resisting GTPPs-induced apoptosis is further demonstrated by the survival of the metastatic oral squamous cell carcinoma OSC2 cells transfected with p57 sense cDNA. None of the parental OSC2 cells (Examples 1 and 2), p57 antisense-transfected, and green fluorescent protein (GFP)-transfected cells survived in 0.2 mg/ml GTPPs. This concentration is not higher than that of green tea drink preparations (Yang et al., Cancer Epidemiol Biomarkers Prev, 1999; 8:83-9) but is lethal to many tumor cell lines. Only the p57 sense cDNA transfected OSC2 cells survived in this GTPPs concentration.

In conclusion, the data presented in this example indicate that p57 plays a crucial and determinant role in cell survival during GTPPs or EGCG challenge.

### Example 4

### Green Tea Polyphenol Targets the Mitochondria in Tumor Cells Inducing Caspase 3 Dependent Apoptosis

GTPPs or EGCG alone or at concentrations found in green tea drink preparations (300-600 μM for EGCG, 0.38-0.76 mg/ml for the four major polyphenols), are able to induce apoptosis in oral squamous carcinoma cells, while normal human epidermal keratinocytes survived (see Examples 1 and 3). EGCG-induced apoptosis involves Apaf-1 and caspase 3, two key factors in the mitochondria-mediated apoptosis pathway (see Example 3). However, whether caspase 3 plays a determinant role is unknown; since other apoptotic pathways might be involved, for example, TNF alpha or Fas induced-death receptor pathway and autophagy pathway (Leist and Jaattela, Nat Rev Mol Cell Biol, 2001; 2:589-98). Elucidation of GTPPs-induced specific apoptosis pathway is crucial to future chemopreventive or therapeutic intervention designs utilizing GTPPs, since certain tumor cells may be resistant to GTPPs. To examine the role of caspase 3 in GTPPs-induced apoptosis, MCF7 (caspase 3 null) cells, which are resistant to caspase 3-executed apoptosis but are able to execute caspase 3-independent apoptosis (Bacus et al., Oncogene 2001; 20:147-55, Cuvillier et al., Cell Death Differ 2001; 8:162-71, Kagawa et al., Clin Cancer Res 2001; 7:1474-80) were used.

The tumor cells selected for this investigation either express wild-type caspase 3 (OSC2, MCF7 caspase 3 +), or are caspase 3 null (MCF7). The OSC2 cell line was isolated from submandibular lymph node metastasis of a 68-year old female, the primary tumor being located in the gingiva of this patient. MCF7 cells were obtained from American Type Culture Collection (ATTC HTB22). MCF7 cells are defective in caspase 3-executed apoptosis and show a lack of downstream events, for example, DNA fragmentation, cellular shrinkage, and blebbing, due to a deletion in the caspase 3 gene (Janicke et al., J Biol Chem 1998; 273:9357-60). MCF7 caspase 3 +cells were generated by stable caspase 3 cDNA transfection of MCF-7 cells. The defective functions described above were restored in these cells (Blanc et al., Cancer Res 2000; 60:4386-90). A concentration gradient of EGCG and 0.2 mg/ml GTPPs was tested for the apoptotic effect in the three tumor cell lines. Pooled human neonatal epidermal keratinocytes were used as negative control for caspase 3 activation. As previously shown in Examples 1 and 3, these normal cells are able to survive in GTPPs through a p57 mediated pathway described previously.

Materials and Methods

Chemicals. EGCG was purchased from Sigma (St. Louis, Mo.). A mixture of four major GTPPs was purchased from LKT Lab. Inc (Minneapolis, Minn.). GTPPs and EGCG were dissolved in cell culture medium and filter-sterilized immediately prior to use. 50 μM of EGCG equals 22.9 μg/ml.

Cell lines and cell culture. The normal human keratinocytes (NHEK CC-2507) were purchased from Cambrex (East Rutherford, N.J.) and maintained in KGM-2 medium (Cambrex). The OSC2 cell line was previously described (Osaki et al., Eur J Cancer B Oral Oncol 1994; 30 B:296-301). The breast carcinoma MCF7 cell line was purchased from American Type Culture Collection. The MCF7(C) caspase+cell line “7-3-28 ” was established and tested as previously described (Janicke et al., J Biol Chem 1998; 273:9357-60, Blanc et al., Cancer Res 2000; 60:4386-90). These tumor cells were maintained in 45% Dulbecco's Modified Eagle's Medium (DMEM), 45% Ham's F12 medium and 10% fetal calf serum, 100 I.U/ml penicillin, 100 μg/ml streptomycin and 5 μg/ml hydrocortisone. All cell cultures were maintained in a 37° C. incubator with 5% CO₂. Light microscopic photographs were taken with a SPOT RT digital camera system (Diagnostic Instruments) linked to a Nikon Phase Contrast-2 microscope at an original magnification of 200×.

Caspase 3 activity assay. The Caspase 3 Apoptosis Detection Kit was purchased from Santa Cruz Biotech. Inc. In a 24 well tissue culture plate, 10⁵ cells/well of cells in triplicates were plated. After 24 hour treatments with EGCG and GTPPs, the cells in each well were washed with 1 ml PBS and incubated with 100 μl lysis buffer on ice for 10 minutes. To each well, 100 μl of 2×reaction buffer was added with 10 μM DTT. Finally, 5 μl of DEVD-AFC substrate was added to each well containing cell lysates. The reaction mixtures were incubated for 1 hour at 37° C. The caspase 3 activity in each well was measured using a fluorescence microplate reader set for 405 nm excitation and 505 nm emission.

MTT (3-(4,5-dimethylthiazol-2-yl)-2,5-diphenyl tetrazolium bromide) assay. This method detects the activity of mitochondrial succinate dehydrogenase (SDH). In a 96-well plate, 1.5×10⁴ cells were seeded in each well. After variety of treatments, 100 μl of 2% MTT was added to each well and the plate was incubated at 37° C. for 30 minutes. 100 μl of 0.2 M Tris (pH 7.7) with 4% formalin was added to each well. After incubation at room temperature for 5 minutes, liquid was removed and the wells were allowed to dry. Each well was rinsed with 200 μl water followed by addition of 100 μl DMSO (6.35% 0.1 N NaOH in DMSO) to each well. The coloration was measured by a Thermo MAX microplate reader (Molecular Devices Corp. Sunnyvale, Calif. using wavelengths of 562 nm. Experiments were repeated three times with triplicate samples for each experiment.

DNA synthesis analysis using BrdU incorporation method. The BrdU cell proliferation kit was purchased from Oncogene Research Products, Boston, Mass. Cells were culture in 96 well plates with 10⁴ cells/well. After EGCG and GTPPs treatments, cells were labeled by BrdU, reacted with BrdU antibody and the color reaction was carried out according to the protocol provided by the manufacturer. The coloration was measure by a Thermo MAX microplate reader using wavelengths 450 nm-562 nm. Experiments were repeated three times with triplicate samples for each experiment.

Cell growth assay. MCF7 cells were seeded (5×10⁴) in 25 cm² tissue culture flask for 24 hours prior to EGCG or GTPPs incubation. Cells from each flask were trypsinized and counted at each time point on a hemacytometer.

Results are based on three repeated experiments.

Results and Discussion

Morphological analysis. Light microscopy photos indicated that both EGCG and GTPPs induced apoptosis only in caspase 3+MCF7 (C) cells. MCF7(C) cells exhibit differential morphology when compared with MCF7 cells. EGCG treatment for 48 hours significantly reduced the cell number and produced cell blebbing, a characteristic of caspase 3 dependent apoptosis (Blanc et al., Cancer Res 2000; 60:4386-90). After MCF7 (C) cells were exposed to 0.2 mg/ml GTPPs for 24 hours, the majority of the cells were either exhibited apoptosis or became fragmented. When incubation with 0.2 mg/ml GTPPs extended to 48 hours, no viable MCF7 (C) cells remained. In contrast, caspase 3 null MCF7 cells did not exhibit reduction in cell density nor cell death after 24 hours GTPPs treatment, while increased cell density was observed in 48 hours EGCG treated cells compared to 24 hour control cultures, indicating that cell growth was not inhibited during EGCG treatment.

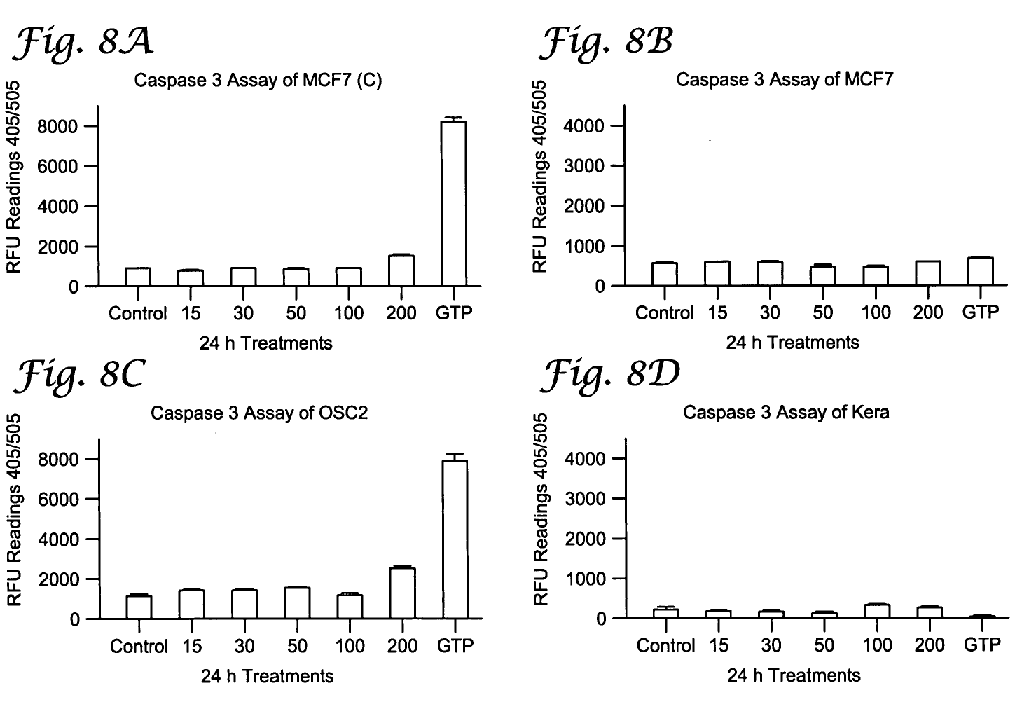

Caspase 3 activity assay. When increasing concentrations of EGCG and 0.2 mg/ml GTPPs were incubated with MCF7 and MCF7 (C) cells, MCF7 (C) demonstrated activated caspase 3 detected by PARP cleavage-based caspase 3 activity assay (FIG. 8A). The pattern of caspase 3 activation was very similar to that of OSC2 cells, which served as a positive control (FIG. 8C). Both OSC2 cells and MCF7 (C) cells were efficiently induced to apoptosis by GTPPs in 24 hours in morphological analysis (current results and results shown in Examples 1 and 3). On the contrary, MCF7 cells did not show caspase 3 activity (FIG. 8B) compared to the normal human epidermal keratinocytes (FIG. 8D), which served as a negative control and was protected by a p57/KIP2-mediated survival pathway (Hsu et al., General Dentistry, 2001; 50:140-146, Pan et al., J Agric Food Chem 2000; 48:6337-46).

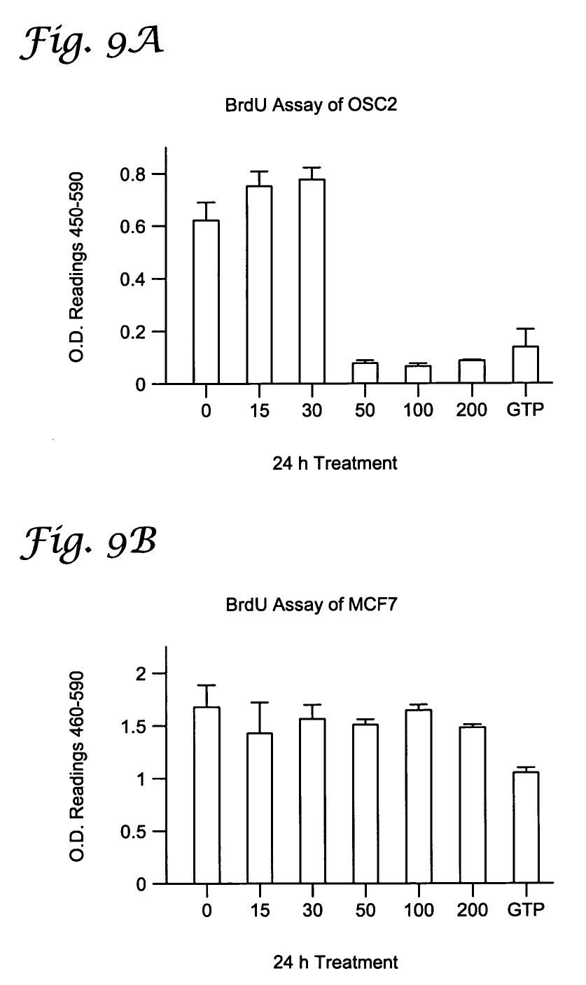

BrdU assay. OSC2 cells were used in the BrdU incorporation assay as positive growth inhibition control (FIG. 9). OSC2 cells ceased BrdU incorporation when EGCG concentrations reached 50 μM (FIG. 9A), while MCF7 cells were able in incorporate BrdU efficiently except in 0.2 mg/ml, where the incorporation decreased, but was not diminished (FIG. 9B).

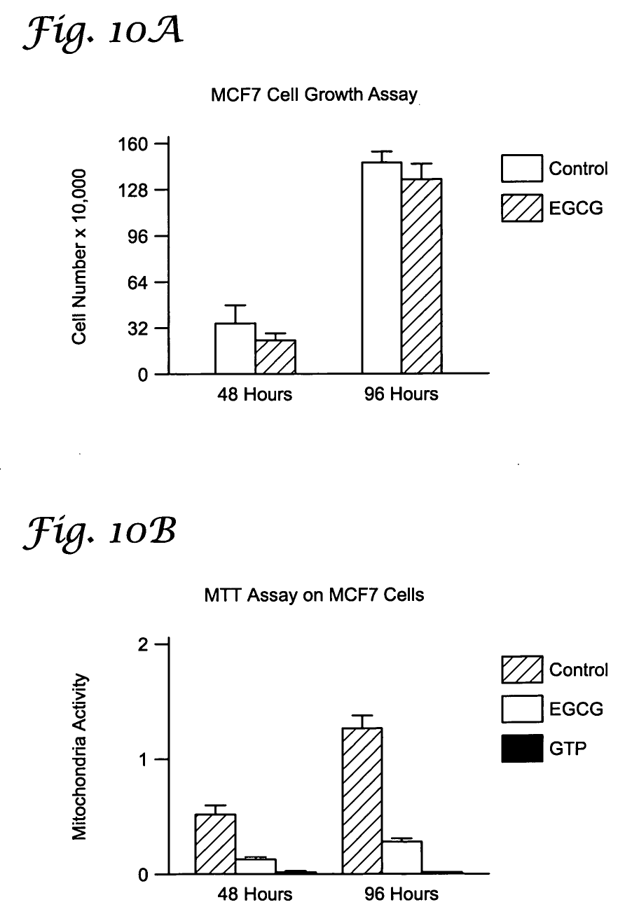

Cell growth assay and MTT assay. Continued culturing of MCF7 cells in 50 μM EGCG for 96 hours showed only an insignificant decrease in cell number compared to untreated cultures (FIG. 10A). However, mitochondrial SDH activities were significantly decreased when MCF7 cells were treated with 50 μM EGCG (FIG. 10B). When MCF7 cells were cultured in the presence of 0.2 mg/ml GTPPs, the SDH activities were completely diminished at 48-hour time point (FIG. 10B).

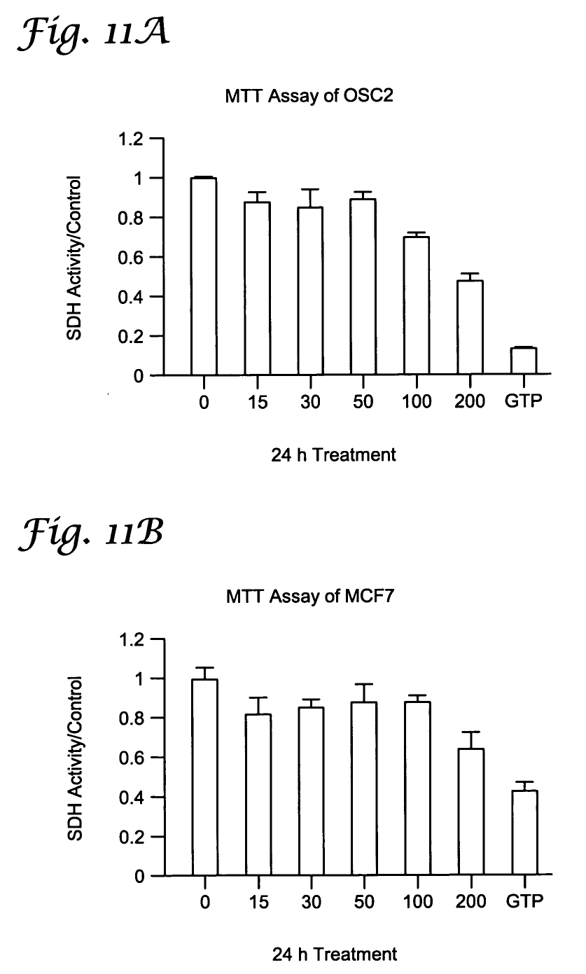

The SDH activities in OSC2 cells decreased during a 24 hour period when exposed to increasing concentrations of EGCG and 0.2 mg/ml GTPPs (FIG. 11A). The caspase 3 null MCF7 cells showed similar patterns when identical treatment was applied (FIG. 11B).

Previous reports have indicated that tea polyphenols induced apoptosis in various tumor cell types, associated with caspase 3 activation. In this regard, EGCG induced caspase 3 dependent apoptosis in human chondrocarcoma cells (Islam et al., Biochem Biophys Res Commun, 2000; 270:793-7). Oolong tea (a form of semi-fermented tea in which polyphenols are partially preserved) polyphenol theasinensin A induced apoptosis in the human histocytic lymphoma cell line U937 through cytochrome c release and activation of caspase-9 and caspase-3 (Pan et al., J Agric Food Chem 2000; 48:6337-46). Cytochrome c release and caspase activation were also observed in Ehrlich ascites tumor cells when exposed to green tea extract (Kennedy et al., Cancer Lett 2001; 166:9-15). These data suggested that GTPPs-induced apoptosis in cancer cells correlated with cytochrome c release and caspase 3 activation.

The current example was designed to address three key questions: one, whether wild type caspase 3 is required for GTPPs-induced apoptosis; two, how normal human epithelial cells respond to the GTPPs treatment in terms of caspase 3 activation; and, three whether GTPPs diminishes the mitochondrial activity in the absence of wild type caspase 3. Results obtained in this example indicate that caspase 3 is a determinant factor for GTPPs-induced apoptosis. GTPPs at the concentration of 0.2 mg/ml was able to eliminate the majority of MCF7 (C) cells in 24 hours, while the parental caspase 3 null MCF7 cells did not exhibit any morphological alterations. Similar patterns were observed when 50 μM EGCG was applied for 48 hours. Lack of wild type caspase 3 in MCF7 cells maintained their survival due to lack of caspase 3 activity (FIG. 8B), while caspase 3+MCF7 (C) cells activated caspase 3 as much as 9 fold (FIG. 8A), which correlated with apoptotic morphology. The caspase 3 activities of MCF7 and MCF7(C) cells were verified by using OSC2 cells as a positive control (FIG. 8C) and human epidermal keratinocytes as a negative control (FIG. 8D). These results strongly indicate that wild type caspase 3 is the executer for GTPPs-induced apoptosis. Therefore the absence of any element in caspase 3 dependent apoptosis pathway could result in resistance to GTPP-induced apoptosis.

The caspase 3 null MCF7 cells were not only resistant to GTPP-induced apoptosis, but also demonstrated continued growth in the presence of 50 μM EGCG for up to 96 hours (see, for example, FIG. 10A). These results, in addition to data from the BrdU assay (FIG. 9B), suggest that EGCG is not able to induce growth arrest in MCF7 cells giving the fact that MCF7 cells possesses wild type p53, and the TGF beta and insulin signaling pathways are intact (Blagosklonny et al., Cancer Res 1995; 55:4623-6, van der Burg et al., J Cell Physiol 1988; 134:101-8, Arteaga et al., Cancer Res 1988; 48:3898-904). The interesting finding is that while MCF7 cells survived GTPPs/EGCG exposure and continue to proliferate, the mitochondria function was gradually impaired by either EGCG or GTPPs initiated at 24 hours (FIG. 10B), and completely depleted by GTPPs in 48 hours (FIG. 11B). This indicates that EGCG at 50 μM concentration is not able to completely eliminate the mitochondrial function (FIG. 11B) and the energy supply for cell proliferation could be provided for the period up to 96 hours. Data from this study and previous investigations indicate that green tea polyphenols target the mitochondria, leading to cytochrome c release and apoptosome formation, and subsequently activate the caspase 3 dependent apoptosis pathway. Cancer cells lacking wild type caspase 3 may be resistant to GTPPs to undergo immediate apoptosis, but the mitochondria could be damaged in a prolonged time period. As shown in Example 3, p57 is a determinant factor for cells survival during GTPPs treatment using either p57 inducible human epidermal keratinocytes or retroviral-transfected OSC2 cells expressing wild type p57.

In this example tumor cells either with deleted caspase 3 gene or expressing wild type caspase 3 were treated by increasing concentrations of green tea polyphenol(s), followed by morphological analysis and caspase 3 activity assay. The caspase 3 null parental cell line was further examined in comparison with a well-characterized, caspase 3 wild type oral carcinoma cell line by MTT assay and BrdU incorporation assay. The results demonstrated that, while the mitochondrial function was gradually declined to insignificant levels, caspase 3 null cells did not undergo apoptosis, suggesting that green tea polyphenol-induced apoptosis is a mitochondria-targeted, caspase 3 executed mechanism.

### Example 5

### Tea Polyphenols Induce Differentiation and Proliferation in Epidermal Keratinocytes

As shown in the previous examples, the green tea polyphenol epigallocatechin-3-gallate (EGCG) induces differential effects between tumor cells and normal cells. Nevertheless, how normal epithelial cells respond to the polyphenol at concentrations for which tumor cells undergo apoptosis is undefined. Thus, the current example tested exponentially growing and aged primary human epidermal keratinocytes in response to EGCG or a mixture of the four major green tea polyphenols. EGCG elicited cell differentiation with associated induction of p57/KIP2 within 24 hours in growing keratinocytes, measured by the expression of keratin 1, filaggrin and transglutaminase activity. Aged keratinocytes, which exhibited low basal cellular activities after culturing in growth medium for up to 25 days, renewed DNA synthesis and accelerated energy production up to 37-fold upon exposure to either EGCG or the polyphenols. These results indicate that tea polyphenols can be used for treatment of wounds or certain skin conditions characterized by altered cellular characteristics.

Example 1 showed that both GTPPs and EGCG are able to induce transient expression of p57/KIP2, a differentiation/cell cycle regulator, which was associated with cell survival during GTPP exposure. It is proposed that p57 induction stimulates cell differentiation as part of a survival pathway. While this survival pathway is currently under investigation, the impact of GTPPs on epidermal keratinocytes located in various layers of the skin was deemed essential to be addressed, given the fact that GTPPs are able to penetrate the epidermis, but not the dermis, of human skin (Dvorakova et al., Cancer Chemother Pharmacol., 1999; 43:331-5).

Keratinocytes within the epidermis exist in various stages of differentiation corresponding to different epidermal layers. For example, the basal keratinocytes and/or stem cells at the dermal-epidermal junction continuously proliferate to regenerate and restore cells lost to the environment. As the daughter cells migrate up through the epidermal layers, they first undergo growth arrest followed by expression of keratins 1 and 10 in the spinous layer. In the next layer, the granular layer, late markers of keratinocyte differentiation, including filaggrin and other structural proteins, are- expressed. In addition, the activity of transglutaminase, the enzyme that cross links the structural proteins into the cornified envelope, is increased. Finally, the keratinocytes undergo an epidermal-specific programmed cell death to form the cornified layer, which serves as a barrier to mechanical injury, microbial invasion and water loss. The entire epidermis turns over in one to two months, although the transit time of keratinocytes may be lengthened or shortened in various disease states. It is pertinent to investigate whether GTPPs induce differential effects among keratinocytes at different stages of differentiation and/or age, knowing that if so, such effects could be significant for assessing the potential impact of these compounds upon topical application. Thus, agents that accelerate growth and/or differentiation of epidermal keratinocytes may shorten the healing time of certain wounds and serve as treatments for conditions such as aphthous ulcers and other epidermal-skin diseases.

In this example, it is shown that green tea polyphenols, either in a mixture or in the form of purified EGCG, are able to increase cellular activities, including new DNA synthesis, in aged keratinocytes, or promote differentiation of exponentially growing keratinocytes located in the basal layer of epidermis. In the current example, pooled normal human primary epidermal keratinocytes treated with EGCG or GTPPs after various times of culture. Results from this study demonstrated that: one, by promoting ATP production and new DNA synthesis, both EGCG and GTPPs “re-energized” the aged keratinocytes; thus, these compounds can presumably stimulate the regeneration of keratinocytes in aging skin; and, two, by induction of p57, keratin 1 and filaggrin expression, and activation of transglutaminase, EGCG also stimulated the differentiation of the keratinocytes found in the basal layer of the epidermis. The combination of these two effects may help to accelerate wound healing and regeneration of new skin tissue, and subsequently prevent scar tissue formation. In addition, certain epithelial conditions may be amenable to treatment by topical applications of green tea polyphenols.

Material and Methods

Chemicals and antibodies. EGCG was purchased from Sigma (St. Louis, Mo.). A mixture of four major green tea polyphenols (GTPPs) was purchased from LKT Lab, Inc (Minneapolis, Minn.). GTPPs and EGCG were dissolved in keratinocyte growth medium-2 (KGM-2, Cambrex) and filter-sterilized immediately prior to use. The rabbit anti-human p57 antibody C-19 was purchased from Santa Cruz Biotechnology (Santa Cruz, Calif.); the rabbit anti-filaggrin and anti-keratin- 1 antibodies were from Covance (Berkeley, Calif.).

Culturing normal human epithelial cells. The pooled normal human primary epidermal keratinocytes were purchased from Cambrex (Baltimore, Md.) and sub-cultured in the specific growth media provided by the manufacturer (KGM-2). Subculture of the epithelial cells was performed by detaching the cells in 0.25% trypsin and transferring into new tissue culture flasks, at the recommended density of 3500 cells/cm². Exponentially growing keratinocytes were treated and harvested in their early passages (2-3 passages). Aged keratinocytes were allowed to grow in 96-well tissue culture plates for 15, 20, and 25 days prior to treatment by EGCG or GTPPs, followed by various assays.

MTT (3-(4,5-dimethylthiazol-2-yl)-2,5-diphenyl tetrazolium bromide) assay. This method detects the activity of mitochondrial succinate dehydrogenase (SDH). In a 96-well plate, 1.5×10⁴ cells were seeded in each well. After 24-hour treatment, culture medium was removed and replaced with 100 μl of 2% MTT in a solution of 0.05 M Tris, 0.5 mM MgCl₂ 2.5 mM CoCl₂, and 0.25 M disodium succinate (Sigma, St. Louis, Mo.) and the plate was incubated at 37° C. for 30 minutes. Cells were fixed in situ by the addition of 100 μl of 4% formalin in 0.2 M Tris (pH 7.7), and after a 5 minute incubation at room temperature liquid was removed and the wells were allowed to dry. Each well was rinsed with 200 μl water and cells were solubilized by the addition of 100 μl of 6.35% 0.1 N NaOH in DMSO. The colored formazan product was measured by a Thermo MAX micro plate reader (Molecular Devices Corp. Sunnyvale, Calif.) at a wavelength of 562 nm. Experiments were repeated three times with triplicate samples for each experiment.

Analysis of DNA synthesis using the BrdU incorporation method. The BrdU cell proliferation kit was purchased from Oncogene Research Products (Boston, Mass.). Cells were cultured in 96-well plates at the density of 10⁴ cells/well. After EGCG and GTPPs treatments, cells were labeled with BrdU for 12 hours and levels of BrdU incorporation determined according to the manufacturer's instructions using a Thermo MAX micro-plate reader at a wavelength of 450 nm and subtracting absorbance measured at 562 nm. Experiments were repeated three times in triplicate for each experiment.

Immunocytochemistry. Normal human keratinocytes were seeded in 8-well chamber slides (Nagle Nunc International, Naperville, Ill.) 12 hours prior to EGCG treatment. At the end of a 24-hour treatment, the slides were washed with PBS and fixed in a cold 4% paraformaldehyde solution for 10 minutes. Then 3% hydrogen peroxide solution and normal goat serum were applied to block endogenous peroxidase activity and non-specific binding. The primary antibodies, rabbit-anti-human p57 polyclonal antibody C-19, rabbit anti-human keratin 1, and filaggrin antibodies were applied for 1 hour at 37° C. at the dilutions recommended by the manufacturers. The streptavidin detection technique (Biogenex, USA) was used with 3-amino-9-ethylcarbazole as chromogen. Negative control sections consisted of tissues treated with 1% diluted normal goat serum instead of primary antibody. Mayer's hematoxylin was used as a counter-stain.

Transglutaminase activity assay. Normal human epidermal keratinocytes in early passages (2-3) were allowed to grow in 6-well tissue culture plates prior to EGCG exposure. The cells were scraped in homogenization buffer (0.1 M Tris/acetate, pH 8.5, containing 0.2 mM EDTA, 20 μM AEBSF, 2 μg/mL aprotinin, 2 μM leupeptin and 1 μM pepstatin A), collected by centrifugation and subjected to one freeze-thaw cycle prior to lysis by sonication. Unlysed cells were pelleted by centrifugation and aliquots of the supernatant collected for the determination of transglutaminase activity and protein concentration. Protein quantities were determined using the BioRad Protein Assay with bovine serum albumin as standard. Transglutaminase activity was measured as the incorporation of [³ H] putrescine into dimethylated casein, as described previously (Jung et al., J Invest Dermatol, 1998; 110:318-23).

Caspase 3 activity assay. The Caspase 3 Apoptosis Detection Kit was purchased from Santa Cruz Biotech., Inc. Cells (10⁵ per well) were plated in triplicate in a 24-well tissue culture plate. After 24 hour treatments with EGCG or GTPPs, the cells in each well were washed with 1 ml PBS and incubated with 100 μl lysis buffer on ice for 10 minutes. To each well, 100 μl of 2×reaction buffer was added with 10 mM DTT. Finally, 5 μl of DEVD-AFC substrate was added to each well containing cell lysates. The reaction mixtures were incubated for 1 hour at 37° C., and caspase 3 activity in each well was measured using a fluorescence micro-plate reader at a wavelength of 405 nm for excitation and 505 nm for emission.

Results and Discussion

As shown in Examples 1-3, unlike a variety of tumor cell types tested, normal human epidermal keratinocytes were able to survive when exposed to EGCG or GTPPs. This survival ability may be due to a differential intracellular response when normal keratinocytes are exposed to EGCG or GTPPs. The mechanism of the survival pathway may involve regulation of pro-survival factors, cell cycle factors and/or cell differentiation factors at the transcriptional and/or translational level. In addition, responses of aged keratinocytes may differ from those of exponentially growing keratinocytes.

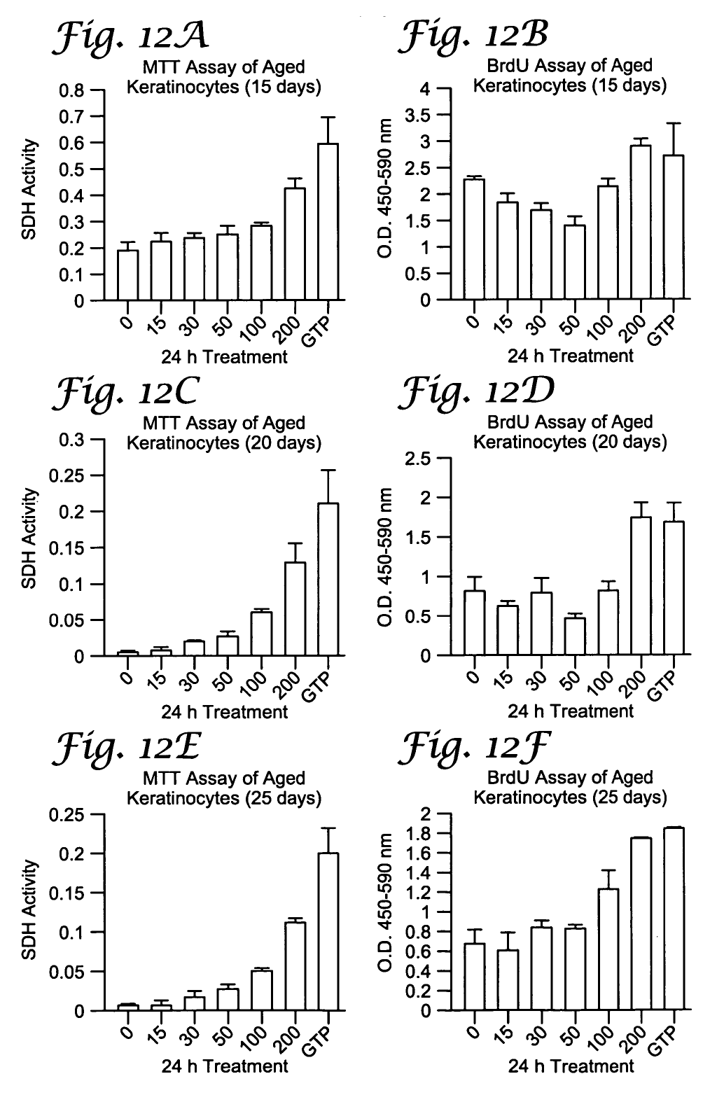

In this example, pooled primary human epidermal keratinocytes, after 15, 20, or 25 days in culture, gradually lost their ability to either generate ATP or divide. At these time points, EGCG or GTPPs were able to activate the mitochondrial enzyme succinate dehydrogenase (SDH), as measured by the MTT assay (FIG. 12A, FIG. 12C, and FIG. 12E), up to 37 fold (25 days, FIG. 12E). The activation of this component of the tricarboxylic acid (TCA) cycle may provide biological energy and substrates for other responses such as new DNA synthesis. When aged human keratinocytes lost the ability to synthesize new DNA, especially after 20 +days in KGM-2, both EGCG and GTPPs were able to stimulate new DNA synthesis, as measured by BrdU incorporation assay (FIG. 12B, FIG. 12D, and FIG. 12F), up to approximately 3 fold (25 days, FIG. 12F). This represents the first observation that green tea components stimulate energy generation and DNA replication in aged epidermal keratinocytes. It was noted that for the aged keratinocytes at the 15-day and 20-day time points, lower concentrations of EGCG (15-50 μM) had a slight negative impact on BrdU incorporation (FIG. 12B and FIG. 12D). On the other hand, EGCG concentrations higher than 100 μM consistently induced both SDH activity and BrdU incorporation (FIG. 12). Therefore, the age of the keratinocytes and the concentration of EGCG or GTPPs used are two key factors in terms of the effects of these agents on energy generation and DNA replication. Of interest is the relationship of aged cultures of keratinocytes to their differentiation status. Since human keratinocytes are prone to undergo growth arrest and to express differentiation markers upon attaining confluence (Lee et al., J Invest Dermatol., 1998;111:762-6), it is predicted that the response of keratinocytes in upper epidermal layers will mirror that of the aged keratinocytes. Thus, EGCG and the GTPPs will stimulate reentry into the cell cycle in the early-differentiated (spinous) stratum of the skin.

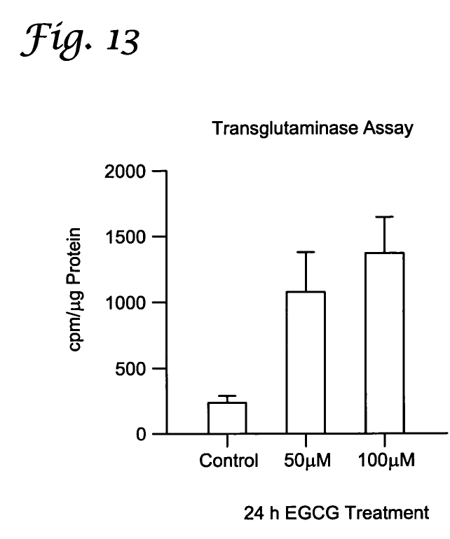

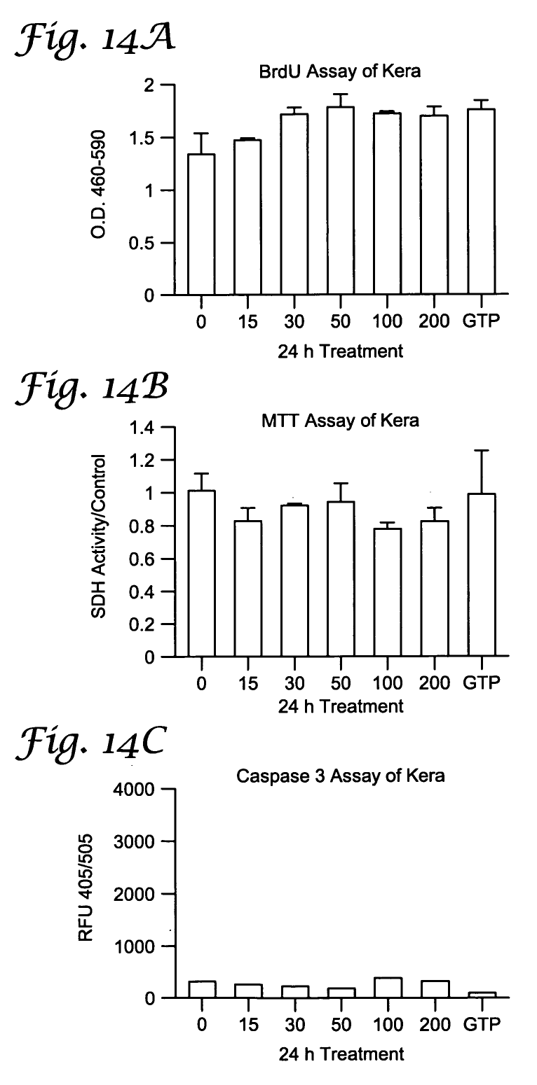

Previous data showing either growth arrest or differentiation of keratinocytes were based on observations in exponentially growing cells for which EGCG enhanced the expression of involucrin and increased the conversion of undifferentiated keratinocytes into corneocytes with concomitant growth arrest (Balasubramanian et al., J Biol Chem., 2002; 277:1828-36). The current study further confirmed that the undifferentiated keratinocytes were able to commit to differentiation upon EGCG treatment within a short period of time, accompanied by an elevation in the activity of transglutaminase, the enzyme that cross-links involucrin and other substrates to form the cornified envelope (Bikle et al., Mol Cell Endocrinol, 2001; 177:161-71). When exponentially growing pooled normal human primary epidermal keratinocytes were incubated with 50-100,μM EGCG, these cells underwent differentiation in 24 hours, as measured by immunocytochemistry using antibodies against human p57/KIP2 (a differentiation/growth arrest inducer), keratin 1 (an early differentiation marker), filaggrin (a late differentiation marker), and transglutaminase activity assay (a late differentiation marker) (FIG. 13). Note also that exposure to EGCG induced an increase in the number of enlarged, flattened, squame-like cells observed in these cultures. This morphology is typical of differentiated keratinocytes, providing further confirmation of the ability of EGCG to trigger cell differentiation. EGCG concentrations of 50-100 μM were adequate to induce cell differentiation and were accompanied by a marked p57 elevation, indicating p57 may not only be responsible for cell survival but also for cell differentiation (see Examples 1-3). The EGCG concentrations used are within the physiological range in humans (Chen et al., Arch Pharm Res., 2000;23:605-12, Jin et al., J Agric Food Chem, 2001; 49: 6033-8, Nakagawa et al., Biochem Biophys Res Commun., 2002; 292:94-101, Nie et al., Arch Biochem Biophys, 2002; 397:84-90, Suganuma et al., Cancer Res., 1999; 59:44-7, Yokoyama et al., Neuro-oncol., 2001; 3:22-8), given the fact that after drinking preparations equivalent to two to three cups of green tea, EGCG secreted from human saliva, excluding other polyphenols, was measured at concentrations up to approximately 50 μM (22.9 μg/ml) (Yang et al., Cancer Epidemiol Biomarkers Prev., 1999; 8:83-9). An in vivo study showed that daily topical application of 30 mg/ml EGCG (655 times higher than 100 μM) for 30 days failed to induce dermal toxicity (Stratton et al., Cancer Lett, 2000; 158: 47-52). In addition, the viability of the keratinocytes was confirmed by BrdU incorporation and SDH activity upon EGCG or GTPP-exposure, and their apoptotic status investigated by a caspase 3 activity assay; there was no major alteration in these measurements (FIG. 14). In order to assess whether increasing polyphenol concentrations themselves alter and/or interfere with the BrdU and MTT assays, an oral carcinoma cell line, OSC2, was treated identically. As shown in Example 4, both BrdU incorporation and MTT levels decreased significantly. This result suggests that the effect of EGCG or GTPPs on exponentially growing keratinocytes is a selective induction of differentiation, in contrast to the apoptotic cell death initiated in OSC2 tumor cells.

Thus, Example 5 shows, for the first time that, at certain concentrations, EGCG or a mixture of the major green tea polyphenols stimulated aged keratinocytes to generate biological energy and to synthesize DNA, available for renewed cell division. For keratinocytes in an exponential growth phase, EGCG or a mixture of the major green tea polyphenols potently stimulated these cells to commit to differentiation with minimal impact on DNA synthesis or energy levels. Stimulating differentiation of keratinocytes in the basal layer of the epidermis and energizing and stimulating cell division/DNA synthesis in aged keratinocytes could potentially reduce the time of healing and prevent the formation of scar tissue, which occupies the space not repopulated by keratinocytes. Therefore, green tea components may be useful topically for promoting skin regeneration, wound healing or treatment of certain epithelial conditions such as aphthous ulcers, psoriasis and actinic keratosis. In addition, the differentiation-inducing potential of green tea components might be beneficial to patients who have conditions characterized by abnormally accelerated skin cell growth and lack of differentiation.

### Example 6

### Green Tea Polyphenol Causes Differential Oxidative Environments in Tumor Versus Normal Epithelial Cells

Recently, cytotoxic reactive oxygen species (ROS) were identified in tumor and certain normal cell cultures incubated with high concentrations of the most abundant GTPP, (−)-Epigallocatechin-3-gallate (EGCG). If EGCG also provokes the production of ROS in normal epithelial cells, it may preclude the topical use of EGCG at higher doses. This example examined the oxidative status of normal epithelial, normal salivary glandular, and oral carcinoma cells treated with EGCG, using (ROS) ROS measurement and catalase and superoxide dismutase (SOD) activity assays. The results demonstrated that high concentrations of EGCG induced oxidative stress only in tumor cells. In contrast, EGCG reduced ROS in normal cells to background levels. MTT assay and BrdU incorporation data were also compared between the two oral carcinoma cell lines treated by EGCG, which suggest that difference in the levels of endogenous catalase activity may play an important role in reducing oxidative stress provoked by EGCG in tumor cells. It is concluded that pathways activated by GTPPs or EGCG in normal epithelial versus tumor cells create different oxidative environments, favoring either normal cell survival or tumor cell destruction. This finding will lead to applications of naturally occurring polyphenols to enhance the effectiveness of chemotherapy and/or radiation therapy to promote cancer cell death while protecting normal cells.

Green tea polyphenols (GTPPs) found in the tea plant (Camellia sinensis), either as a mixture or as the most abundant GTPP, (−)-Epigallocatechin-3-gallate (EGCG), induce apoptosis in many types of tumor cells, and have been proposed as chemopreventive or therapeutic agents (Stoner and Mukhtar, J Cell Biochem Suppl, 1995; 22:169-180; Lambert and Yang, Mutat Res, 2003; 523-524:201-208). Green tea constituents have been characterized as antioxidants that scavenge free radicals to protect normal cells (Higdon and Frei, Crit Rev Food Sci Nutr, 2003; 43:89-143; Bors et al., Arch Biochem Biophys, 2000; 374: 347-355; Wei et al., Free Radic Biol Med, 1999; 26:1427-1435; Ruch et al., Carcinogenesis, 1989; 10:1003-1008; Lee et al., Chem Biol Interact, 1995; 98:283-301; Huang et al., Carcinogenesis, 1992; 13:947-954; Katiyar et al., Toxicol Appl Pharmacol, 2001; 176: 10-117; and Katiyar et al., Carcinogenesis, 2001; 22: 287-294). However, recent reports have linked GTPPs to reactive oxygen species (ROS) production, especially hydrogen peroxide (H₂O₂), and subsequent apoptosis in both transformed and non-transformed human bronchial cells (Yang et al., Carcinogenesis, 2000; 21:2035-2039). ROS are normal by-products of aerobic metabolism. Most intracellular ROS are generated via mitochondrial electron transport, although other normal biological processes contribute. To maintain a proper redox balance, many defense systems have evolved. A major cellular defense against ROS is provided by superoxide dismutase (SOD) and catalase, which together convert superoxide radicals first to H₂O₂, and then to water and molecular oxygen. Other enzymes such as glutathione peroxidase and thioredoxin reductase use the thiol reducing power of glutathione and thioredoxin, respectively, to reduce oxidized lipid and protein targets of ROS. H₂O₂ has been detected when a colon adenocarcinoma HT29 cell line was incubated with EGCG (Hong et al. Cancer Res, 2002; 62:7241-7246). It has been suggested that, in a human B lymphoblastoid cell line, concentrations of EGCG higher than physiological levels (10 μM) induced the production of ROS, especially H₂O₂, which inflict damage (Sugisawa and Umegaki, J Nutr, 2002; 132:1836-1839). In an immortalized normal breast epithelial cell line (MCF10 A), EGCG induced growth arrest prior to the cell cycle restriction point, with elevated p21, hypophosphorylation of Rb and decreased cyclin Di, suggesting that higher concentrations (50-200 μM) of EGCG found in green tea may be toxic to normal mammary epithelial cells (Liberto and Cobrinik, Cancer Lett, 2000; 154:151-161). Example 3 demonstrated the apoptotic effect of EGCG on human primary mammary epithelial cells, in which 50 μM EGCG induced apoptosis 24-96 hours after treatment. Although the apoptosis-inducing factor(s) in these normal cells is(are) unknown, a trend was evident: normal cells originating from the epidermis, oral cavity and digestive tract are tolerant of high doses of the polyphenols, while cells from elsewhere show sensitivity to high concentrations of GTPPs.

Examples 1-5 described differential responses of normal epidermal keratinocytes versus certain tumor cells to GTPPs, and proposed that GTPPs activate multiple pathways in different cell types. This may apply to the oxidative status imposed by GTPPs or EGCG in various cell types. Primates closely related to humans rely predominantly on fresh leafy plants for their energy needs. If humans maintained a diet similar to their ancestors, an adult human would consume approximately 10 kg of fresh leafy plant food daily to meet daily energy requirements (Milton, Nutrition, 1999; 15:488-498). Many leafy plants, either fruits or vegetables, have high levels of the polyphenols/tannins (Bravo, Nutr Rev, 1998; 56: 317-333; Nepka et al., Eur J Drug Metab Pharnacokinet, 1999; 24:183-189). Primates, including humans, may have evolved a tolerance to exposure to tannin-rich plants. It is hypothesized that cells in frequent contact with plant-derived polyphenols, such as cells found in the epidermis, oral mucosa and digestive tract, have developed mechanism(s) to mitigate the toxicity and benefit from these compounds.

However, GTPPs, when applied in high doses, are cytotoxic to other human cells that lack this tolerance and to cancer cells that have lost these protective mechanisms. In this example, EGCG concentrations up to 50 times higher than the maximum plasma concentration (Cmax) were tested on human oral carcinoma cells, normal epidermal keratinocytes and immortalized normal salivary gland cells. The results demonstrate that EGCG at high concentrations failed to produce ROS and in fact lowered ROS to background levels in these normal cells. In contrast, the oral carcinoma cells, which respond to GTPPs by undergoing apoptosis, elevated ROS levels upon treatment in a dose-dependent manner. The ROS levels were significantly higher in the cell line that possesses low catalase activity, and their persistence was extended. These observations suggest that EGCG is able to create differential oxidative environments in normal epithelial versus tumor cells by exploiting compromised redox homeostasis in the tumor cells.

Material and Methods

Cell lines. Pooled normal human primary epidermal keratinocytes (NHEK) were obtained from Cambrex Corporation (Baltimore, Md.) and maintained in KGM-2 medium (Cambrex Corporation). The OSC-2 and OSC-4 cell lines, were cultured in Dulbecco's Modified Eagle's Medium (DMEM)/Ham's F12 50/50 mix medium (Cellgro, Kansas City, Mo.) supplemented with 10% (volume/volume (v/v)) fetal bovine serum, 100 I.U./ml penicillin, 100 μg/ml streptomycin and 5 μg/ml hydrocortisone. OSC-2 and OSC-4 cells have one mis-sense mutation (exon 8, codon 280: AGA→ACA) and one silent mutation (exon 5, codon 174: AGG→AGA) in the p53 gene, respectively (Yoneda et al., Eur J Cancer, 1999; 35:278-283). Immortalized normal salivary gland cells (NS-SV-AC), selected following transfection of origin-defective SV40 mutant DNA, were maintained in KGM-2 medium (Azuma et al., Lab Invest, 1993; 69: 24-42).

Reagents. EGCG, 3-(4, 5-dimethylthiazol-2-yl)-2, 5-diphenyl tetrazolium bromide (MTT), catalase and diamide were purchased from Sigma-Aldrich (St. Louis, Mo.). Dihydrofluorescein diacetate (DFDA) and SOD were obtained from Molecular Probes Inc. (Eugene, Oregon) and ICN Biomedicals Inc. (Aurora, Ohio), respectively.

Measurement of intracellular ROS levels. The ROS assay measures the accumulation of intracellular ROS levels. The non-fluorescent dye DFDA passively diffuses into cells, where the acetates are cleaved by intracellular esterases. The metabolites are trapped within the cells and oxidized by ROS, mainly H₂O₂, to the fluorescent form, 2′, 7′-dichlorofluorescein, which can be measured by fluorescent plate reader to reflect levels of intracellular ROS (mainly H₂O₂). Thus, values of the fluorescence in the cell cultures are constantly rising in this assay. Cells (1.5×10⁴ cells/well) were incubated with Hallam's physiological saline (HPS) containing DFDA (10 μM) in a 96-well microplate for 30 minutes at 37° C. After the incubation, cells were washed three times with HPS and then incubated with HPS containing EGCG (15-200 μM) or diamide (5 mM) for the indicated time periods. The intracellular ROS levels were measured by using a fluorescence plate reader (BIO-TEK FL600, Bio-Tek Instruments, Inc., Winooski, Vt.), at an excitation wavelength of 485 nm and an emission wavelength of 530 nm.

DNA synthesis assay. DNA synthesis was analyzed by a BrdU Cell Proliferation Assay Kit (Oncogene Research Products, Boston, Mass.). Briefly, cells (1×10⁴ cells/well) were seeded in a 96-well microplate and treated with the indicated doses of EGCG for 24 hours at 37° C. After the treatment, cells were labeled with BrdU for 2 hours at 37° C. and reacted with anti-BrdU antibody. Unbound antibody in each well was removed by rinsing, and horseradish peroxidase-conjugated goat anti-mouse antibody was added to each well. The color reaction was visualized according to the protocol provided by the manufacturer. The color reaction product was quantified using a Thermo MAX microplate reader (Molecular Devices Corp., Sunnyvale, Calif.) at dual wavelengths of 450-540 nm.

MTT [3-(4,5-dimethylthiazol-2-yl)-2,5-diphenyl tetrazolium bromide] assay. This method directly detects the activity of mitochondrial succinate dehydrogenase (SDH). Changes in SDH activity is a measurement of cell viability when stress is introduced in cell culture through chemical or physical means. Cells (1.5×10⁴ cells/well) were seeded in a 96-well microplate and treated with the indicated doses of EGCG for 24 hours. After the treatment, the cells in each well were washed with 200 μl of phosphate-buffered saline (PBS), incubated with 100 μl of 2% MTT in a solution of 0.05 M Tris, 0.5 mM MgCl₂, 2.5 mM CoCl₂, and 0.25 M disodiumn succinate as substrate (Sigma) at 37° C. for 30 minutes. Cells were fixed in situ by the addition of 100 μl of 4% formalin in 0.2 M Tris (pH 7.7), and after a 5 minute incubation at room temperature liquid was removed and the wells were allowed to dry. Each well was rinsed with 200 μl water and cells were solubilized by the addition of 100 μl of 6.35% 0.1 N NaOH in DMSO. The colored formazan product was measured by a Thermo MAX micro plate reader (Molecular Devices Corp., Sunnyvale, Calif.) at a wavelength of 562 nm. Experiments were repeated three times with triplicate samples for each experiment.

Assays for SOD and catalase activities. Cells (1×10⁶ cells/well) were incubated with or without EGCG (50 μM) in FilterCap 50 ml flasks (Nagel Nunc International, Rochester, N.Y.) for 30 minutes at 37° C. After the incubation, cells were harvested and disrupted in 100 μl of 10 mM Tris-HCl (pH 7.4) containing 0.1%(v/v) Triton X-100, 10 μg/ml leupeptin, 10 μg/ml pepstatin A and 100 mM phenylmethylsulfonyl fluoride by three cycles of freezing/thawing. After centrifugation at 17,000×g for 20 minutes at 4° C., the supernatants were used for SOD and catalase assays using the SOD Assay Kit-WST (Dojindo Molecular Technologies, Inc., Gaithersburg, Md.) and the AMPLEX Red Catalase Assay Kit (Molecular Probes), respectively. The activities of SOD and catalase were calibrated using a standard curve prepared with purified human SOD and catalase. The activities of SOD and catalase were expressed as units (U)/10⁶ cells.

Statistical analysis. All data are reported as mean±SD. A one-way ANOVA and unpaired Student's t tests were used to analyze statistical significant. Differences considered statistically significant at p<0.05.

Results and Discussion

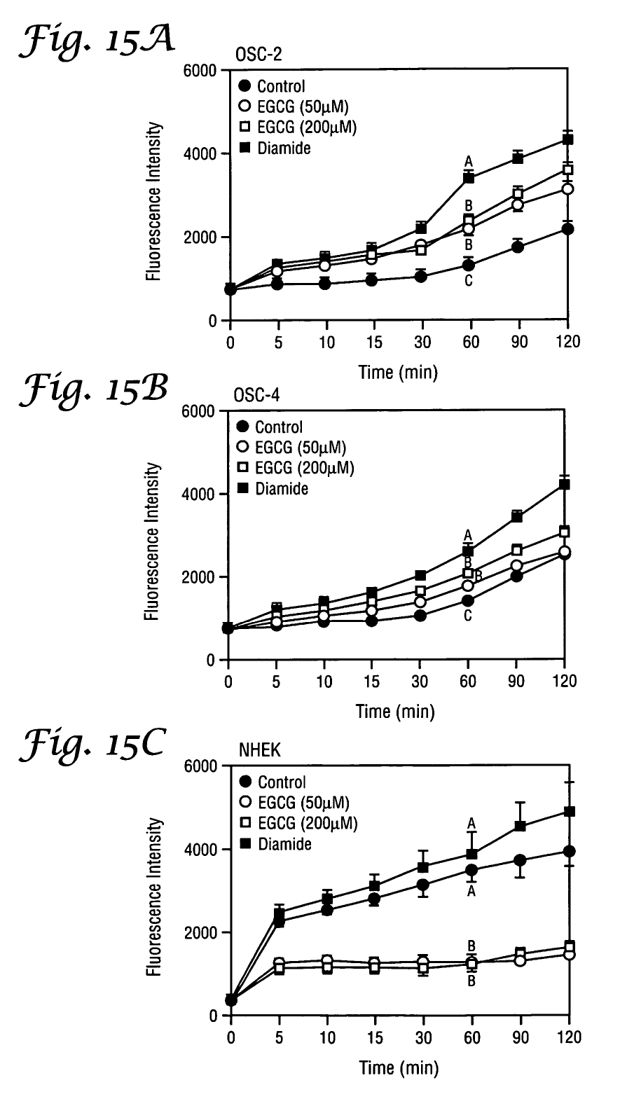

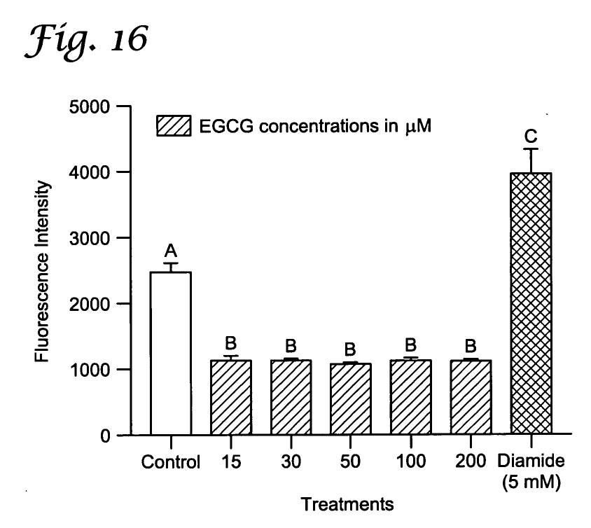

ROS assay. FIG. 15A shows that ROS levels similar to those induced by diamide were generated in OSC-2 cells immediately after the addition of 50 or 200 μM EGCG into the cell culture and matched diamide's levels up to 15 minutes. After this period, diamide-induced ROS levels increased at a faster rate than EGCG-induced levels. At 60 minutes, an EGCG dose response was detectable, with 200 μM EGCG inducing higher levels of ROS than 50 μM treatments. The EGCG-induced ROS levels remained significantly higher than the control levels beyond the 120 minute time point, but lower than the ROS levels produced by diamide. In OSC-4 cells, an EGCG dose response was apparent 10 minutes after EGCG was applied (FIG. 15B). As found in OSC-2 cells, EGCG-generated ROS levels rose at a similar rate to that of diamide-induced ROS throughout the first 15 minutes post-exposure. Beyond 15 minutes, the diamide-induced ROS levels increased at a faster rate than the EGCG-induced levels. The rate of ROS production in OSC-4 cells incubated with EGCG peaked at 60 minutes, and then decreased to less than either diamide-treated or untreated controls. Thus, at the 120 minute time point, 50 μM EGCG treated cells had ROS levels identical to the control cells, while ROS in 200 μM EGCG-treated cells remained higher than the control cells. For NHEK, diamide induced ROS in the cells after 1-minute incubation when compared to the endogenous ROS levels (FIG. 15C). In contrast to OSC-2 or OSC-4 cells, the ROS levels in NHEK were significantly reduced immediately after the addition of EGCG, and the ROS maintained at basal levels throughout the testing period of 120 minutes. In addition, there was no apparent EGCG dose effect in these normal cells. In NS-SV-AC cells, EGCG at various concentrations was also able to inhibit ROS production at background levels when measured at the 60 minute time point (FIG. 16).

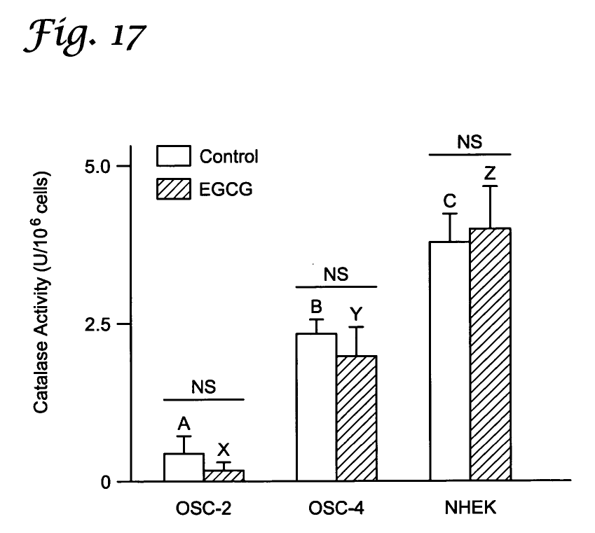

Catalase activity assay. Significant changes in catalase activity was not observed in any cell type when these cells were treated with 50 μM EGCG for 30 minutes. However, significant differences in the levels of endogenous catalase activity were found among the three cell types. NHEK had the highest endogenous catalase activity (per 10⁶ cells), OSC-4 cells showed moderate levels of catalase activity, while OSC2 cells exhibited the lowest levels of catalase activity (FIG. 17).

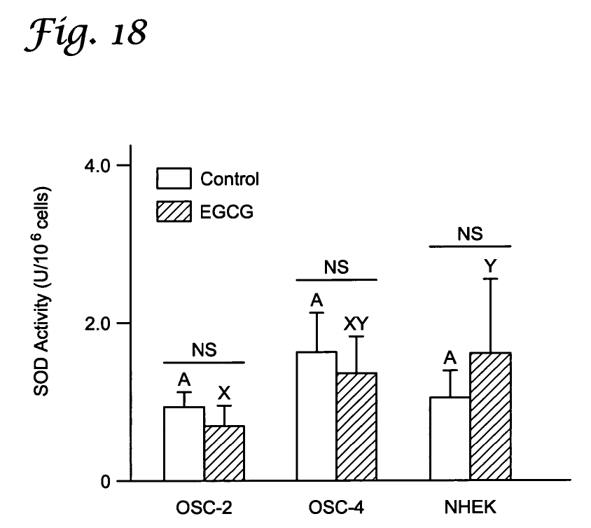

SOD activity assay. All three cell types possess significant amounts of SOD activities (FIG. 18). Incubation with 50 μM EGCG for 30 minutes did not alter SOD activity in any of the cell types.

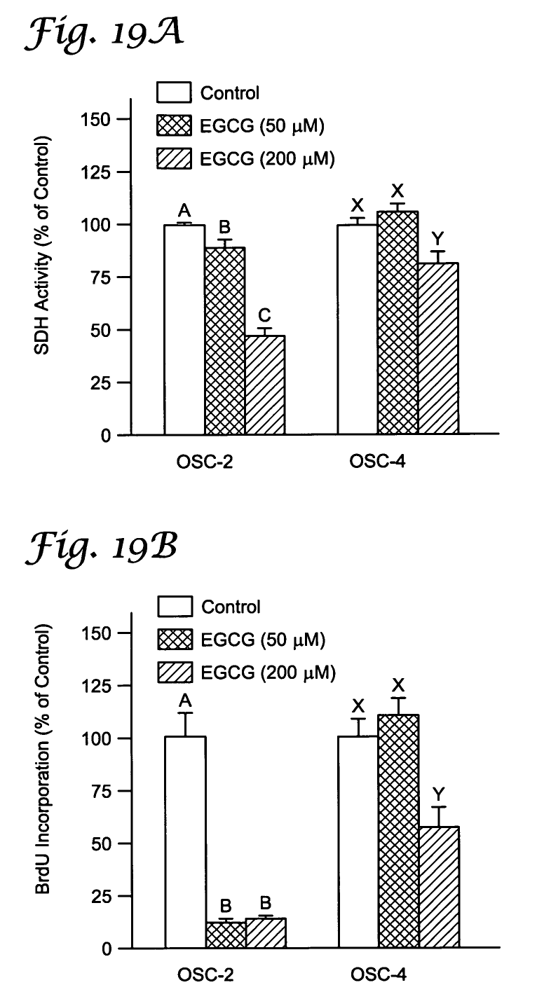

MTT and BrdU assays. OSC4 cells did not show significant changes in the mitochondrial SDH activity (as measured by MTT assays, FIG. 19A) and DNA synthesis (measured by the BrdU assay, FIG. 19B) following incubation with 50 μM EGCG for 24 hours. However, when EGCG concentration increased to 200 μM, OSC4 cells demonstrated significantly reduced SDH activity and DNA synthesis. In comparison to SDH activity and DNA synthesis in EGCG-treated OSC2 cells, (shown in Example 4), where 50 μM EGCG reduced both SDH activity and DNA synthesis, OSC4 cells appeared less sensitive to EGCG.

Previous reports have suggested that EGCG at high concentrations produces ROS, especially H₂O₂, in cell cultures (Yang et al., Carcinogenesis, 2000; 21:2035-2039; Sakagami et al., Anticancer Res, 2001; 21:2633-2641; and Chai et al., Biochem Biophys Res Commun, 2003; 304: 650-654). The current findings confirmed this observation from two oral carcinoma cell lines, which demonstrated the formation of intracellular ROS when incubated with EGCG in a dose-dependent manner (FIG. 15A and 15B). According to previous reports, EGCG-induced ROS formation can also occur in certain normal cells.

However, the current study demonstrated that high concentrations of EGCG (up to 200 μM) failed to induce ROS formation in normal epidermal keratinocytes cultured in growth media. In contrast, intracellular ROS levels in these EGCG-treated normal cells persistently decreased to, and were maintained at, insignificant levels. Conversely, ROS levels in the untreated cultures continued to climb, at rates near those of diamide-treated cell cultures (FIG. 15C). These results demonstrated that EGCG might act as a ROS inducer or a strong ROS scavenger, depending upon specific cell type. Whereas it appears that the concentrations of EGCG used might play a role in the rate of production of ROS in tumor cells, normal epithelial cells were able to tolerate very high concentrations of EGCG (approximately 50 times higher than the Cmax in plasma) and reduce ROS to background levels five minutes after EGCG was added in the culture, regardless of concentration (15-200 μM). In the previous examples, it was proposed that GTPPs or EGCG activate multiple pathways, depending upon cell types. The differential effects of GTPPs or EGCG in normal epithelial versus tumor cells signal the tumor cells to undergo apoptosis but direct the normal epithelial cells toward a survival pathway associated with cell differentiation (Examples 4 and 5). Results from the current example identified the differential impact of EGCG on oxidative status in normal versus tumor cells, indicating that GTPPs are cytotoxic to human cells that have not developed a tolerance for tannins/polyphenols, such as tumor cells and cells from internal organs, whereas cells in potentially frequent contact with plant-derived compounds are tolerant to, and possibly benefit from, GTPPs in high concentrations. One potential mechanism might be the association of GTPP/EGCG sensitivity to the loss of the ability of a tumor cell to differentiate, regardless of the origin of the tumor.

Results from the catalase activity assay demonstrated that the NHEK possess the highest levels of catalase activity per cell among the cell types examined and EGCG had no effect on this activity (FIG. 17). This high level of catalase activity could be part of a defense system specific to the epithelial cells designed to eliminate H₂O₂ produced by environmental factors, such as radical-producing agents and ultraviolet light, in this case, diamide (FIG. 17C). In the tumor cell lines, endogenous catalase activity in OSC-2 cells was the lowest. This observation correlated with the high ROS levels produced by EGCG both initially and sustained in OSC-2 cells (FIG. 15A). The cause for the low activity of catalase in OSC-2 cells may due to low catalase protein produced by these cells. In this regard, it is expected that OSC-2 cells would be more sensitive to oxidant-induced DNA damage, mutation or apoptosis since catalase is a major scavenger for H₂O₂. OSC-4 cells showed moderate levels of catalase activity (FIG. 17) and produced less ROS than OSC-2 cells (FIG. 15A and 15B). The protein levels of catalase in each cell type are consistent with the activity measurements. This result may explain why OSC-4 cells are more resistant to GTPP/EGCG-induced cytotoxicity when compared with OSC-2 cells, as reflected by the reduced effect of these agents on mitochondrial SDH activities and BrdU incorporation (FIG. 19). In contrast, identical conditions of EGCG treatment did not significantly alter levels of the SDH activity or BrdU incorporation in NHEK (Example 5).

OSC-2 cells possess a defective p53 pathway due to a gene mutation (Yoneda et al., Eur J Cancer, 1999; 35:278-283), which may contribute to their susceptibility to GTPP/EGCG-induced apoptosis (Examples 1 and 2). It was reported previously that H₂O₂ is able to induce apoptosis in certain tumor cells, and addition of exogenous catalase completely eliminated this apoptotic effect (Yang et al., Carcinogenesis, 1998; 19:611-616). Interestingly, normal rat aorta responded to EGCG by phasic contraction, which was triggered by EGCG-induced H₂O₂ but not superoxide, possibly propelled by H₂O₂ triggered Ca++ release (Shen et al., Clin Exp Pharmacol Physiol, 2003; 30:88-95). Human embryonic kidney 293 cells also respond to EGCG with H₂O₂ production in a dose-dependent pattern (Dashwood et al., Biochem Biophys Res Commun, 2002; 296:584-588). The evidence suggested that formation of H₂O₂ occurs when cells from internal organs are exposed to EGCG.

Inhibition of SOD in tumor cells was reported in human promyelocytic leukemia HL-60 cells, which was associated with apoptosis (Zhang et al., Anticancer Res, 2002; 22:219-224). On the other hand, activation of SOD was found in normal large intestine of GTPP or EGCG-fed rat (Yin et al., Cancer Lett, 1994; 79:33-38), suggesting that the EGCG effect on SOD activity is cell-type specific. In this example, all three cell types showed moderate levels of SOD activities (FIG. 18). Compared to catalase activity, SOD activity appeared to be a relatively insignificant factor in ROS scavenging capacity when the cells were incubated with EGCG for 30 minutes. This may due to the formation of EGCG-induced ROS in the tumor cells were mainly in the form of H₂O₂, which depends on catalase for its elimination. Nevertheless, whether EGCG differentially regulate catalase and SOD on transcription/translation levels in epithelial cell systems remain to be investigated.

Many studies suggest that antioxidant systems are critical in protecting against tumor promoting agents, and that one or more components of these systems are deficient in many forms of cancer. This observation is logical, given the fact that DNA is a major target of oxidative stress and accumulation of DNA damage contributes to tumor formation. Both catalase and manganese SOD (Mn-SOD) appear to be particularly important in this regard. Several studies found catalase deficiencies in a variety of tumors, as well as in cells derived from patients with the DNA-repair defective disease xeroderma pigmentosa (Vuillame et al., Carcinogenesis, 1992; 13:321-328). In addition, hypocatalasemic mice were protected against breast tumor formation by vitamin E supplementation, supporting an oxidative component in mammary tumor development (Ishii et al., Jpn J Cancer Res, 1996; 87:680-684). It was previously showed ROS-induced apoptosis in tumor cells could be rescued by Mn-SOD (Ueta et al., Jpn J Cancer Res, 1999; 90:555-564; and Ueta et al., Int J Cancer, 2001; 94:545-55). Likewise, overexpression of Mn-SOD can reduce oxidative DNA damage and alter transcription regulation, leading some to propose it as a new type of tumor suppressor. The mechanism responsible for this suppressor function remains unclear, but several studies report that activation of redox-sensitive transcription factors (i.e. NF-kB, AP1, Nrf2) is altered by changes in Mn-SOD levels (Kiningham and St Clair, Cancer Res, 1997; 57:5265-5271). GTPPs belong to the phenolic flavonoid class of antioxidants which recently have been proposed to act as electrophiles that can activate MAPK pathways through an electrophilic-mediated stress response, and activate the phase 2 gene-inducing transcription factor, Nrf2 (Rushmore and Kong, Curr Drug Metab, 2002; 3:481-490). Thus, EGCG may serve as an important modulator of certain transcription factors to regulate intracellular redox status.

EGCG is rapidly absorbed through the oral mucosa in humans and secreted back into the oral cavity by saliva, suggesting that salivary glandular cells may be tolerant of high concentrations of EGCG (Yang et al., Cancer Epidemiol Biomarkers Prev, 1999; 8:83-89). The current example supports this concept by data from incubating various concentrations of EGCG (15-200 μM) with a SV40-immortalized normal human sublingual salivary acinar cell line (FIG. 16). Consistent with data obtained from human epidermal cells (NHEK), EGCG, regardless of the concentration, reduced the ROS to background levels in these cells. Mitochondrial SDH activity in NS-SV-AC cells and two other immortalized normal human salivary glandular cell lines was further tested. The results indicated that these salivary glandular cells were tolerant to high concentrations of EGCG with accelerated energy expenditure.

The current study identified two novel observations. One, EGCG differentially affects oxidative status and can act as either a ROS inducer or ROS suppressor depending upon the cell type, and, two, EGCG concentrations higher than plasma Cmax do not produce H₂O₂ in cells derived from the normal epidermis and oral cavity (and possibly digestive tract), but rather protects these cells by decreasing ROS production. Mechanisms responsible for the differential effects of EGCG could rely on distinctive signal pathways activated by EGCG in a tissue-specific manner that requires further investigation. The knowledge gained from this example will lead to the future use of high concentrations of GTPPs in combination with chemo/radiation therapies in the epidermis, oral cavity and digestive tract, to simultaneously enhance tumor cell death rate and protect normal cells from chemo/radiation-induced oxidative stress. In addition, topical and oral administration of GTPPs, even at low concentrations such as 15 μM, would successfully provide protection against oxidative stress, especially H₂O₂, in such tolerant cells.

### Example 7

### A Mechanism-Based In Vitro Anticancer Drug Screening Approach for Phenolic Phytochemicals

As shown in the previous examples, certain mechanisms underlying the differential effects of green tea polyphenols (GTPPs) on tumor versus normal cells have been determined, indicating that GTPPs may simultaneously activate multiple pathways. However, existing screening methods are insufficient for the identification of agents that possess both a cytotoxic effect on tumor cells and a protective effect on normal cells. This example describes the establishment of an in vitro survival/apoptosis testing system based on detecting these mechanisms by a double-fluorescence method. This system is able to screen potential chemopreventive or therapeutic agents from (but not limited to) plant-derived compounds based on the pathways differentially activated by the agents. Tumor cell death and normal cell survival are detected simultaneously, in a device that co-cultures normal human cells adjacent to human tumor cells.

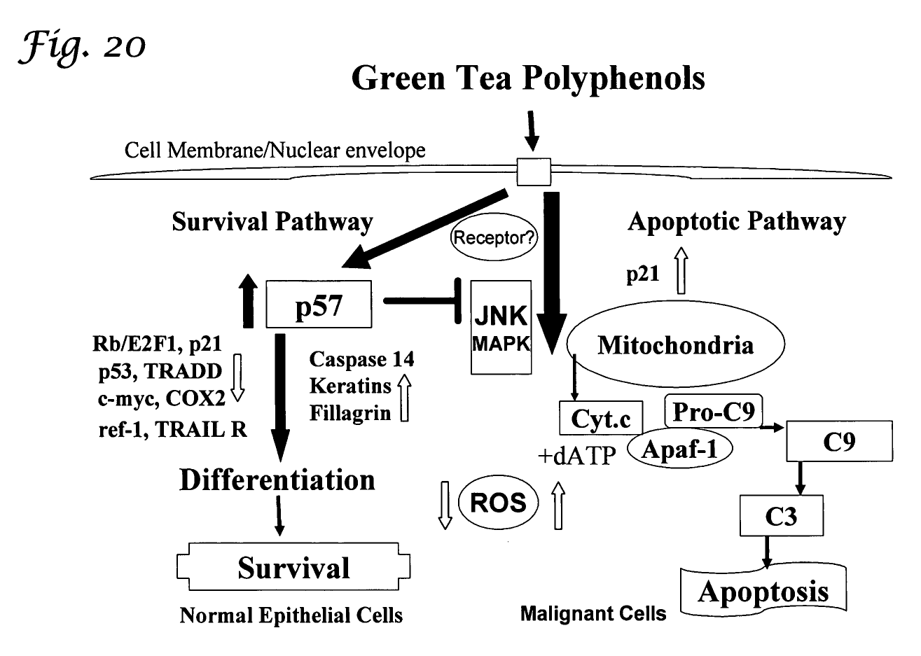

As shown in Examples 1, 3, and 5, induction of p57 by GTPPs in normal epithelial cells is necessary and sufficient for cell survival. In contrast, when exposed to GTPPs, tumor cells lack a p57 response. This results in a caspase 3-dependent apoptosis. GTPPs, either as a mixture or as the most abundant GTPP, (−)-epigallocatechin-3-gallate (EGCG), induce apoptosis in many types of tumor cells (Stoner and Mukhtar, J Cell Biochem Suppl 1995; 22:169-180). Pathology studies demonstrated that tumor specimens express lower levels of p57 protein compared to paired normal tissues, and low levels of p57 often correlate with poor prognosis (Ito et al., Oncology 2001; 61:221-225, Ito et al., Liver 2000; 22:145-149, Ito et al., Pancreas 2001; 23:246-250, Ito et al., Int J Mol Med 2002; 9:373-376). The differential effect of GTPPs/EGCG and signal pathways are summarized in FIG. 20.

Example 4 reported that GTPP-induced apoptosis occurred in various oral carcinoma and breast cancer cells. The GTPP-induced apoptosis is mitochondria-mediated and caspase 3-dependent, as confirmed by caspase 3 activity assay, Annexin V apoptosis assay, and the MTT assay. Importantly, caspase 3 deficient cells are resistant to GTPP-induced apoptosis, but become sensitive after stable transfection with wild type caspase 3 (See Examples 1, 3, and 4). The oral carcinoma cell line OSC-2 showed high sensitivity to GTPPs (see Examples 1, 2, and 4). OSC-2 cells stably transfected with green fluorescent protein (GFP) cDNA, OSC-GFP, maintained the high sensitivity to GTPPs as measured by caspase 3 activity and MTT assays. Importantly, the green fluorescence diminished when OSC-GFP cells were induced to apoptosis by GTPPs or EGCG (Example 3). Thus, both OSC-2 and OSC-GFP lines have been used for detecting activation of apoptosis by GTPPs, including the polyphenol EGCG.

These observations suggest that following exposure to plant-derived phenolics, p57 induction can be used as a marker for cell survival in human epithelial cells, and the activation of the apoptotic pathway (detected by diminished green fluorescence in OSC-GFP cells) can be used as a marker for tumor cell destruction (FIG. 20). This example demonstrates a proof-of-principle for an in vitro co-culture system for anticancer drug screening based on double fluorescent detection of these two pathways activated by for plant-derived phenolic compounds. This system may also be used to test the potency/efficacy of potential or currently available medications or products that possess chemopreventive or therapeutic properties. The unique figure of this system is the ability to detect tumor cell death and normal cell survival in a device in which normal human epithelial cells are co-cultured with human tumor cells. Although several in vitro co-culture systems using paired normal and malignant cells that mimic the in vivo environment have been developed for anticancer drug screening (Appel et al., Cancer Chemother Pharmacol 1986; 17:47-52, El-Mir et al., Int J Exp Pathol 1998; 79:109-115, Torrance et al., Nat Biotechnol 2001; 19:940-945), these systems were not based on intracellular activation of specific pathways, and are not able to mimic human epidermal or mucosal tissues. The advantages of using a co-culture screening system include: one, it more closely resembles the in vivo environment where normal cells and tumor cells are adjacent and interacting; two, it reduces variation caused by separate culture of normal and tumor cells; three, it facilitates elimination of a “false positive” agent, for example, one that kills both tumor and normal cells, which still is a major problem in conventional drug screening; and four, it is able to detect differential pathways activated in normal versus tumor cells.

In the method described here, desirable chemopreventive/therapeutic agents induce apoptosis in the tumor cells (detected by diminished green fluorescence) and induce p57 expression (detected by red fluorescence) in normal cells concomitantly. The effects of an agent can be recorded by simple standard immuno-fluorescence microscopy techniques. This model represents the first co-culture drug screening approach that monitors intracellular pathways for tumor cell destruction and normal cell survival simultaneously. This method has the potential to be modified for high-throughput screening. Therefore, plant-derived compounds, numbered in the tens of thousands (King and Young, J Am Diet Assoc 1999; 99:213-8), could be efficiently screened for their anticancer properties. Further, the principles of the system are adaptable to other pathways and cell lines.

Materials and Methods

Chemicals and antibodies. EGCG was purchased from Sigma (St. Louis, Mo.). A mixture of four major green tea polyphenols (GTPPs) was purchased from LKT Lab, Inc (Minneapolis, Minn.). GTPPs and EGCG were dissolved in keratinocyte growth medium-2 (KGM-2, Cambrex) and filter-sterilized immediately prior to use. The rabbit anti-human p57 antibody and goat anti-rabbit IgG-Rhodamine were purchased from Santa Cruz Biotechnology (Santa Cruz, Calif.).

Cell lines and cell culture. Pooled normal human primary epidermal keratinocytes (NHEK) were obtained from Cambrex Corporation (Baltimore, Md.) and maintained in KGM-2 medium (Cambrex). The OSC-2 cell line was isolated from cervical metastatic lymph nodes of a patient with oral squamous cell carcinoma (Osaki et al., Eur J Cancer B, Oral Oncol 1994; 30B: 296-301), and was cultured in Dulbecco's Modified Eagle's Medium (DMEM)/Ham's F12 50/50 mix medium (Cellgro, Kansas City, Mo.) supplemented with 10%(v/v) fetal bovine serum, 100 I.U./ml penicillin, 100 μg/ml streptomycin and 5 μg/ml hydrocortisone. The human lung diploid fibroblasts WI-38 was purchased from American Type Culture Collection and maintained in F12 medium supplemented with 5% Nu Serum, 125 units/ml penicillin, 125 μg/ml streptomycin, and 10 μg/ml glutamine.

Generation of OSC-GFP cell lines. The GFP cDNA (Clonetech, Palo Alto, Calif.) was subcloned into the HindIII site of the retroviral vector pLNCX2 (Clonetech). Virus was generated in RetroPack PT67 cells (Clonetech) by transfection and antibiotic G418 selection. The transfected PT67 cells were cultured in standard DMEM medium. The viral titer was determined according to the manufacturer's suggestion. OSC-2 cells were transfected by incubation for 24 hours with the virus-containing DMEM medium removed from PT67 culture. The GFP expressing clones were selected by 60 μg/ml G418.

Co-culture and GTPP treatment. Various patterns of co-culture of the tumor/normal cells were achieved by different designs. Examples include:

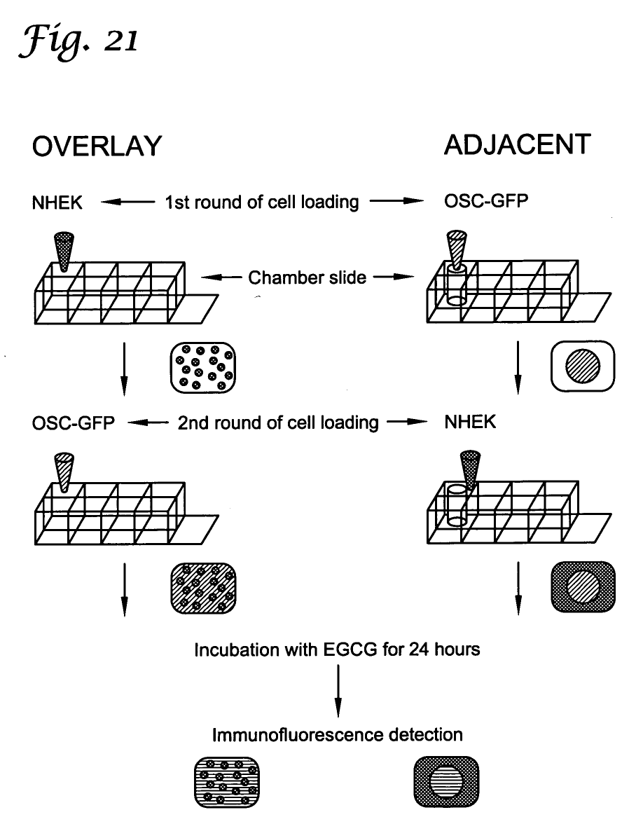

1. Adjacent co-culture design. OSC-GFP cells (5×10⁴) were seeded in the center of a culturing device (8-well chamber-slide, Nagle Nunc International, Naperville, Ill.) through a cloning cylinder (Fisher Scientific/Scienceware, Tapered Design, 4.7×8 mm) in DMEM/F12 medium and allowed to attach for 24 hours. NHEK (10⁵) in KGM-2 were then seeded in the area next to the cylinder. After 24 hours, the cylinder was removed and the medium was replaced by a 50/50 mix of KGM-2 and DMEM/F12 and the cells were allowed to grow for another 24 hours prior to treatment (FIG. 21, right).

2. Overlay design, which could be adapted for high throughput screening in 96 well plate. NHEK (2×10⁴) were seeded in the wells of an 8-well chamber-slide and allowed to grow in KGM-2 medium for 48 hours. OSC-GFP cells (2×10⁵) were then seeded in the wells in DMEM/F12 for 24 hours. Medium was changed to a 50/50 mix of KGM-2 and DMEM/F12 for 24 hours prior to treatment (FIG. 21, left).

3. For monitoring co-culture of fibroblasts adjacent to OSC-2 cells, OSC-GFP cells (5×10⁴) were seeded in the center of wells of an 8 well chamber slide through a cloning cylinder and incubate for 24 hours. Human lung diploid fibroblasts WI-38 (10⁵) were then seeded in the area next to the cylinder in F12 medium and allowed to grow for 24 hours. The cylinder was then removed and the medium was changed to a 25/75 mix of DMEM/F12 for 24 hours prior to GTPP-treatment.

4. Tumor cell migration was monitored by co-culturing OSC-GFP and NHEK in wells of a 24 well plate as indicated in “1”, except the NHEK cell number was doubled. After treatment, tumor cell migration into NHEK territory was recorded by fluorescent microscopy at selected time points. If appropriate, NHEK may be replaced by other normal cells such as WI-38.

Immunofluorescence and photography. At the end of a 24 hour treatment with EGCG, the 8-well chamber slide was washed with PBS and fixed in a 4% paraformaldehyde/PBS solution for 30 minutes at room temperature, followed by washing with PBS three times. The slide was then treated with permeablization solution (0.1% Triton-100, 0.1% sodium citrate) on ice for 2 minutes followed by PBS washing for three times. The slide was incubated in blocking buffer (5% goat serum and 5% BSA in PBS) at 37° C. for 60 minutes. The primary antibody, rabbit-anti-human p57 polyclonal antibody (H 91, Santa Cruz) in PBS/5% BSA, was applied to the samples for 1 hour at 37° C. at the dilution (1:50) recommended by the manufacturer. Negative control sections consisted of cells incubated with 1% diluted normal goat serum instead of primary antibody. After washing three times with PBS, the slide was incubated with the secondary anti-rabbit IgG conjugated with rhodamine (Santa Cruz) for 1 hour at 37° C. Finally, the slide was washed three times with PBS containing 0.1% tween-20, and then mounting solution (Prolong Antifade, Molecular Probes, Eugene, Oreg.) was applied to each well, and the slide was covered by a cover slip. The samples were visualized under a Nikon Phase Contrast-2 microscope. Fluorescent photomicrographs were taken with a SPOT color digital camera system (Diagnostic Instruments) using the ZEISS Axiovert 10 with an original magnification of 200×. Fluorescence was generated by a ZEISS AttoArc 2 source. Light photographs were taken with a SPOT RT digital camera system linked to a Nikon Phase Contrast-2 microscope at an original magnification of 200×.

The total fluorescence intensities of images were quantified using the BIOQUANT NOVA PRIME 6.0 software (Bioquant Co., Nashville, Tenn.). The ratio of rhodamine/FITC reflects the status of the p57-associated survival pathway in NHEK and the apoptosis pathway in OSC-GFP in the normal/tumor co-culture.

Results and Discussion

A retroviral promoter-driven, green fluorescence protein-expressing OSC-2 cell line (OSC-GFP) was generated as described in Example 3. This cell line maintains the parental line's high sensitivity to GTPP-induced apoptosis at concentrations encountered by the oral mucosa (up to 0.3 mg/ml), and shows diminished green fluorescence associated with apoptosis. Results from cell growth and caspase 3 activity assays showed that OSC-GFP cells responded to GTPPs or EGCG similar to the parental OSC-2 cells. When OSC-GFP cells were co-cultured with NHEK cells, as shown by the reduction in green fluorescence, GTPPs induced apoptosis at a level comparable to that seen in OSC-GFP cells alone, indicating co-culture or the mix of culture media did not alter the signal for apoptosis in OSC-GFP cells when exposed to GTPPs. Induction of p57 by EGCG in NHEK grown alone was confirmed by immunofluorescence.

The results of the cell death and survival experiments that GFP-OSC-2 cells grown alone showed bright green fluorescence when cultured without green tea polyphenols. The green fluorescence was lost after 48 hours of exposure to 0.2 mg/ml green tea polyphenols, followed by growth in normal medium for an additional 48 hour. Extensive cell death is apparent only GTPP-treated cells, with morphological changes visible by light microscopy. FITC fluorescent microscopy showing GFP-OSC-2 cells bordering NHEK treated with 0.2 mg/ml GTP for 48 hours, and placed in normal medium for additional 48 hours. Light microscopy of NHEK grown alone after 24 hour treatment with 100 μM EGCG showed no cell death. Rhodamine fluorescent microscopy viewing of NHEK alone treated with 100 μM EGCG for 24 hours followed by immunofluorescence staining with p57 primary antibody and secondary antibody conjugated with rhodamine confirmed the induction of p57.

Co-culture with OSC-GFP cells did not affect the induction. The red fluorescence was therefore used as a cell survival indicator in the co-culture system. When OSC-GFP cells and NHEK were plated in an overlay pattern, EGCG exposure resulted in extensive apoptosis in OSC-GFP cells, leaving large unoccupied spaces. Untreated co-culture cells exhibited strong green fluorescence compared to EGCG-treated co-culture cells, while p57 induction was only detected in EGCG-treated co-culture cells, as indicated by rhodamine (red fluorescence) in NHEK. Green/red merged images demonstrated OSC-GFP cells expressing strong GFP whereas NHEK only express basal p57 without EGCG-exposure. In contrast, EGCG-treated co-culture showed an opposite pattern compared to untreated, strong red fluorescence and diminished green fluorescence, representing simultaneously tumor cell apoptosis and NHEK survival. Quantitative measurement using the BIOQUANT NOVA PRIME 6.0 software showed the ratio of fluorescence intensities of rhodamine (red)/FITC (green) in the control cells was 0.01, while in the EGCG-treated cells it was 1.23. That is, there was a more than 100-fold change in the relative ratios following EGCG treatment.

When OSC-GFP cells were plated adjacent to WI-38 cells, untreated co-culture cells exhibited a defined border between the two cell types observed by either fluorescent microscopy or light microscopy. A clear border was not formed in the co-culture treated with GTPPs due to tumor cell apoptosis. OSC-GFP cells without GTPP treatment were able to expand into the WI-38 occupied area, and did not allow WI-38 cell infiltration. GTPPs caused both OSC-GFP cell apoptosis and WI-38 cell infiltration, seen as elongated fibroblasts.

When OSC-GFP cells were plated adjacent to NHEK, the tumor cells migrated onto the layer of NHEK. The tumor cells reached the edge of the well in 48 hours. In contrast, OSC-GFP cells in GTPP-treated co-culture failed to migrate.

Many leafy plants, either fruits or vegetables, have high levels of phenolic compounds (Bravo, Nutr Rev 1998; 56:317-333, Nepka et al., Eur J Drug Metab Pharmacokinet 1999; 24:183-189). These compounds are part of the plants defense system, acting as pesticides against a variety of organisms. However, primates closely related to humans rely predominantly on fresh leafy plants for their energy needs. It is likely that primates, including humans, may have evolved a tolerance to exposure to these phenolic compounds in the epidermis, oral epithelium and digestive tract.

As shown in Example 6, high concentrations of tea polyphenols (50-600 μM) not only failed to induce cell damage in these tissues, but also provide protection against reactive oxygen species. NHEK have been widely reported to tolerate high concentrations of tea polyphenols (Balasubramanian, J Biol Chem 2002; 277:1828-1836, Fu, Biomed Environ Sci. 2000; 13:170-9). The survival mechanism of NHEK involves cell differentiation associated with p57 induction, which is time and dose-dependent (See Examples 1 and 3). In contrast, normal cells derived from internal organs, such as bronchial, mammary and kidney can be damaged by polyphenols and undergo apoptosis at concentrations higher than the maximal plasma concentration (Example 4).

Caspase 3 positive tumor cells, such as the oral carcinoma lines OSC2 and SCC25, and the breast carcinoma T47D cell line, as well as caspase 3-transfected MCF7 cells, also undergo a caspase 3-dependent apoptosis upon exposure to the polyphenols (Examples 1, 3, and 4). Transfection and expression of p57 cDNA in OSC-2 cells resulted in resistance to GTPP-induced apoptosis (Example 3). Therefore, in the current study, p57 expression was chosen as a marker for activation of a cell survival pathway, whereas well-characterized OSC-2 cells were chosen to reflect polyphenol-induced apoptosis. Other normal/tumor cell systems may also be adapted for drug-screening purposes according to specific needs, but we recommend using normal cells that can be induced by phenolic compounds to express large amount of p57 or caspase 14, a terminal differentiation marker, which is over-expressed after GTPP treatment. Tumor cells that either express high levels of p57 or lack functional caspase 3 should be avoided since they might be resistant to the effects of phenolic compounds (Example 4).

The designs of devices to be used for the co-culture system are very flexible, depending on the purpose of the testing. An eight-well chamber slide was used in the current study for double fluorescence detection; images in the left panel were taken from a single area of untreated co-culture, which shows light microscopy, FITC fluorescence, rhodamine fluorescence and merged fluorescent images. Compared to images of EGCG-treated co-culture cells, the differential effects of EGCG were apparent, especially in the merged images. Combined with paired light microscopy images, interpretation of results can be simplified as: 1) if an agent causes diminished green fluorescence and induced red fluorescence, this agent is able to destroy tumor cells while protecting normal cells; 2) if an agent causes diminished green fluorescence but does not induce red fluorescence, this agent is able to destroy tumor cells but not protect normal cells; 3) if an agent does not cause diminished green fluorescence nor induce red fluorescence, this agent is not able to kill tumor cells or protect normal cells; 4) if an agent induces red fluorescence but does not diminish green fluorescence, this agent is able to protect normal cells but does not destroy tumor cells.

As shown above, there is a large (greater than 100-fold) increase in the relative red:green ratio in the co-cultured wells following EGCG treatment. This large difference represents the simultaneous induction of survival-associated expression of p57 (tagged by rhodamine) and apoptosis-associated reduction of GFP (measured by FITC filter) after the co-culture was treated with 100 μM EGCG (a level well within the range that oral epithelial cells could be exposed to under normal dietary conditions).

One strategy to adapt this approach to high throughput screening will be to use a dual-fluorescence micro-plate reader to quantitatively measure the differential effect of candidate agents in a 96 well plate format, for example, by measurement of the ratio of total rhodamine/FITC fluorescence per well, as described above. The overlay method described above will be the simplest to adapt to this format.

In addition to identifying the differential effects of potential anticancer agents, which promote normal epithelial cells to enter a survival/differentiation pathway and tumor cells to enter an apoptotic pathway, this system is also able to test the impact of a given agent on tumor/normal cell interaction. The untreated co-culture of OSC-GFP and WI-38 cells demonstrated tumor cell expansion toward the fibroblasts. The border area exhibited physical pressure from tumor cells, and there were no fibroblasts found among tumor cells. These characteristics were not observed in GTPP-treated co-culture, where the border was not formed. During the treatment time when the tumor cells underwent apoptosis, the fibroblasts migrated and infiltrated into the area previously occupied by the tumor cells, suggesting cell movement from the opposite direction occurred. What attracted the fibroblast migration is unknown. As shown in Example 4, EGCG inhibited OSC2 cell invasion and migration in transwells without other cell types. The current example confirmed this previous observation. Therefore the co-culture system is adequate to test a given agent for its anti-migration potential by real time monitoring and recording of tumor cell movement comparing to untreated co-culture. In conclusion, this mechanism-based in vitro co-culture system could be used to screen plant-derived phenolic compounds, and other agents, for their differential effects toward apoptosis and survival with simple detection methods and flexible designs. High throughput screening can be achieved with certain modifications. In addition to drug screening, cell interaction and tumor cell migration can be monitored by this system.

### Example 8

### Tea Polyphenol Inversely Regulates Caspase 14 and p21/WAF1 Facilitating Keratinocyte Terminal Differentiation

As shown in the earlier examples, tea polyphenol induces a survival pathway in normal human epidermal keratinocytes (NHEK). This example shows that the tea polyphenol-induced NHEK pathway is associated with induction of caspase-14 and down-regulation of p21/WAF1, linking EGCG to epidermal keratinocyte terminal differentiation, which could be significant in therapy development for certain skin disorders.

Cyclin dependent kinase inhibitor, p21/WAF1/CIP1, plays important roles in cell proliferation, terminal differentiation and apoptosis, although its exact role in keratinocytes is unclear. Increased expression of p21 was associated with a murine keratinocyte calcium-induced differentiation model (Missero et al., Proc Natl Acad Sci. USA. 1995 ; 92:5451-5455; however, overexpression of p21 inhibited murine keratinocyte differentiation marker expression (Di Cunto et al., Science, 1998 ; 280:1069-1072. Immunoprecipitation of p21 from terminally differentiating murine keratinocytes initially demonstrated increased p21 bound to cyclin dependent kinase (cdk)/cyclin D complex (140% of the control at 4 hours), but the complex significantly declined after 8 hours (Martinez et al., Oncogene 1999; 18:397-406. Thus, sustained elevation of p21 levels may be prohibitive for murine keratinocyte terminal differentiation, instead triggering only growth arrest, as previously shown (Dransfield et al., J Invest Dermatol. 2001; 117:1588-1593. Whether p21 expression plays a similar role in human epidermal keratinocytes is not known.

Caspase 14, identified in 1998 from murine tissues (Ahmad et al., Cancer Res. 1996; 58:5201-5205; Hu et al., J Biol Chem. 1998; 273:29648-29653; Van de Craen et al., Cell Death Differ. 1998; 5:838-846), is expressed only in epithelial tissues, especially the epidermis. Unlike the other caspases, caspase 14 is not involved in the well-documented apoptotic caspase cascade, but is associated with terminal keratinocyte differentiation (Lippens et al., Cell Death Differ. 2000; 7:1218-1224; Eckhart et al., J Invest Dermatol. 2000; 115:1148-51; Pistritto et al., Cell Death Differ. 2002; 9:995-1006). Induction of caspase 14 at the transcriptional level was noted during stratum corneum formation (Eckhart et al., Biochem Biophys Res Commun. 2000; 277:655-659). Upon inhibition of cell differentiation, caspase 14 expression was diminished (Rendl et al., J Invest Dermatol. 2002; 119:1150-1155). Therefore, caspase 14 is believed to regulate epidermal differentiation, possibly signaling terminal differentiation and cornification of the epidermis. In contrast, in pathological conditions such as psoriasis, in which cornification does not occur, the expression of caspase 14 is lacking (Lippens et al., Cell Death Differ. 2000; 7:1218-1224).

Examples 1-5 reported that green tea polyphenols selectively induced caspase 3-dependent apoptosis in cells that failed to show p57 induction after EGCG treatment, while normal human epidermal keratinocytes (NHEK) showed elevated p57 expression and underwent differentiation with basal levels of caspase 3. To identify additional factors those suppress EGCG -induced apoptosis and facilitate cell differentiation in NHEK, exponentially growing NHEK (Cambrex, Baltimore, Md.) were exposed to 100 μM EGCG for 0, 2, 6 and 24 hours, prior to total RNA isolation using the Qiagen RNeasy mini kit (Valencia, Calif.), which was followed by RT-PCR labeling and hybridization with the Human Apoptosis Macroarray membrane (Sigma-Genosys, The Woodlands, Tex.). Total cell lysates also were collected following a variety of EGCG treatments. The protein levels for caspase 14 and p21 were determined by immuno-blotting using antibodies specific for caspase 14 and p21 (Santa Cruz Biotechnology, Santa Cruz, Calif.).

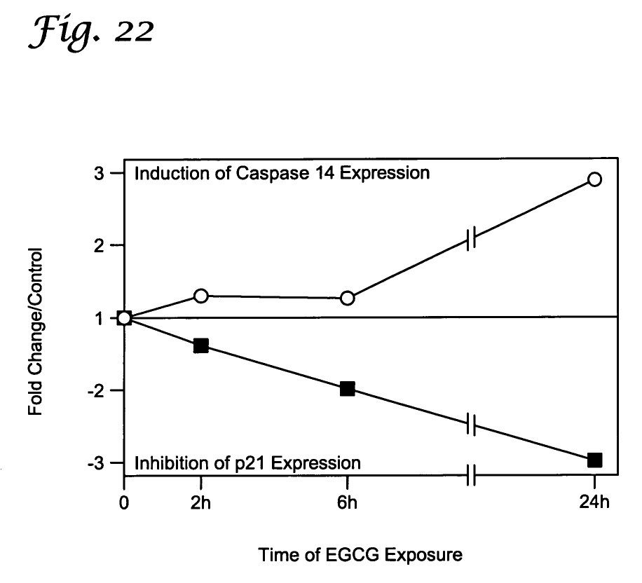

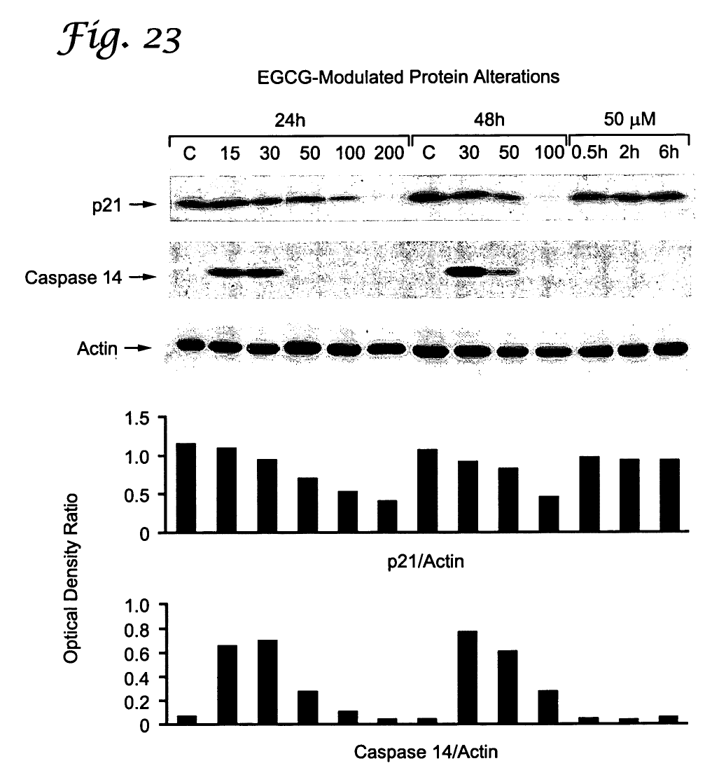

The macroarray results demonstrated that EGCG induced caspase 14 mRNA expression in NHEK, to approximately three fold above control by 24 hours (FIG. 22). Increased transcription was translated to protein levels in whole cell lysates. EGCG at or below 50 μM induced more than a 5 fold increase in caspase 14 protein by 24 hours, and 30 μM EGCG induced a 26 fold increase in 48 hours; EGCG at 100 μM only increased caspase 14 by 2 fold at 24 hours and 5 folds at 48 hours (FIG. 23. Optical Density Ratio), indicating that high concentrations of EGCG were less effective than lower concentrations in inducing caspase 14. Concomitant down-regulation of p21 gene expression occurred in NHEK exposed to EGCG. At 2 hours and 6 hours, mRNA levels were reduced to 70.3% and 50.4% of the untreated control, respectively; at 24 hours, p21 mRNA was only 32.7% of control (FIG. 22). Protein levels of p21 decreased only after 6 hours EGCG treatment; p21 was suppressed to levels less than 50% of controls beyond 24 hours exposure with EGCG concentrations of 100 μM or higher (FIG. 23).

Both caspase 14 and p21 protein levels remained relatively stable during the initial 6 hours, and were altered significantly after that period. In contrast, tumor cells from the oral squamous carcinoma cell line OSC2, which undergo caspase 3-dependent apoptosis when exposed to EGCG (Example 4), failed to show increased caspase 14 or decreased p21 under identical conditions. The results indicate that when NHEK are exposed to EGCG (and/or possibly other phenolic phytochemicals), the exogenous signals are translated intracellularly to direct the keratinocytes toward terminal differentiation, simultaneously protecting the cells from apoptosis.

Therefore, a death-initiating signal from EGCG, and the EGCG-induced oxidative stress, is redirected in NHEK (Examples 5 and 6). Significant induction of caspase 14 occurs after 6 hours treatment, while p57 protein levels peaks at 6 hours. Since p57 is a member of the KIP/CIP family, involved in regulation of cell growth, apoptosis and differentiation (Lee et al., Genes Dev. 1995; 9:639-49; Yan et al., Genes Dev. 1997; 11:973-83; Deschenes et al., Gastroenterology. 2001; 120:423-438), caspase 14 could be a down-stream target of a p57-mediated pathway.

The induction of caspase 14 expression by EGCG in NHEK, supports the differentiation mechanism proposed to explain this naturally protective phenomenon. Thus, green tea constituents may be used not only for chemoprevention, but also for acceleration of epidermal keratinocyte differentiation; by inducing caspase 14 expression, leading to cornification of the epidermis, EGCG may prove useful in treatment of psoriasis, wounds and other skin abnormalities.

### Example 9

### Roles of Catalase and Hydrogen Peroxide in Green Tea Polyphenol-Induced Chemopreventive Effects

The green tea polyphenol-(−) epigallocatechin-3-gallate (EGCG) possesses promising anticancer potential. While in vivo studies unveiled metabolic routes and pharmacokinetics of EGCG and showed no adverse effects, in vitro studies at high concentrations demonstrated oxidative stress. EGCG causes differential oxidative environments in tumor versus normal epithelial cells, but the roles that EGCG, hydrogen peroxide (H₂O₂) and intracellular catalase play in the epithelial system are largely unknown. This example employed enzyme activity assays, reactive oxygen species quantification, BrdU incorporation, and immunoblotting, to investigate whether EGCG-induced differential effects correlate with levels of key antioxidant enzymes and H₂O₂. It was found that normal human keratinocytes with high catalase activity are least susceptible to H₂O₂, while H₂O₂ incurred significant cytotoxicity in oral carcinoma cell lines. However, the EGCG-induced differential effects could not be duplicated by H₂O₂ alone, and the amount of H₂O₂ produced by high concentrations of EGCG was inadequate to cause cytotoxicity in these tumor cells if EGCG was not present. Addition of exogenous catalase failed to completely prevent the EGCG-induced cytotoxicity, and failed to rescue the EGCG-induced growth arrest in the tumor cells. Antioxidant N-acetyl-L-cysteine only rescued the tumor cells from H₂O₂-induced damage but not from EGCG-induced mitochondrial damage. Finally, alterations in catalase or superoxide dismutase activities were not observed upon EGCG exposure. In conclusion, while endogenous catalase may play a role in response to H₂O₂-induced cytotoxicity, the EGCG-induced cytotoxic effects on tumor cells are mainly resulted from sources other than H₂O₂.

Green tea polyphenols (GTPPs), -(−) epigallocatechin-3-gallate (EGCG) in particular, are strong antioxidants (Tanaka, J Toxicol Sci, 2000; 25:199-204; Higdon and Frei, Crit Rev Food Sci Nutr, 2003; 43:89-143). The ability of these compounds to scavenge reactive oxygen species (ROS), such as hydrogen peroxide (H₂O₂) and superoxide radicals, relies on their phenolic chemical structures (Wei et al., Free Radic Biol Med, 1999; 26:1427-1435; Zhu et al., J Agric Food Chem, 2000; 48:979-981). It was suggested that GTPPs, especially EGCG, may help to protect various cells from chemical (such as reactive oxygen species (ROS)) or physical damage (such as ultraviolet light (UV)) that leads to carcinogenesis (Wei et al., Free Radic Biol Med, 1999; 26:1427-1435; Tanaka, J Toxicol Sci, 2000; 25:199-204; Katiyar and Elmets, Int J Oncol, 2001; 18:1307-1313; Chen et al., Toxicol Sci, 2002; 69:149-156; Lee et al., Phytother Res, 2003; 17:206-209). Conversely, GTPPs and EGCG induce cytotoxicity and apoptosis in many types of tumor cell (Lin et al., Biochem Pharmacol, 1999; 58:911-915; Roy et al., Mutat Res, 2003; 523-524:33-41). The EGCG-induced apoptosis has been reported to be associated with oxidative stress imposed on tumor cells, especially by H₂O₂, generated in the cell culture medium by EGCG (Long et al., Free Radic Res, 1999; 31:67-71; Yang et al., Carcinogenesis, 2000; 21:2035-203; Zhu et al., J Agric Food Chem, 2000; 48:979-981).

EGCG-induced production of H₂O₂ was recently observed under in vitro conditions with or without the presence of cells (Long et al., Free Radic Res, 1999; 31:67-71; Hong et al., Cancer Res, 2002; 62:7241-7246). The EGCG-induced oxidative stress triggers an apoptotic pathway that is distinct from chemical or Fas-mediated pathways, and acts through activation of mitogen activated protein (MAP) kinases c-jun N-terminal kinase (JNK) and p38, and the caspase cascade (Kong et al., Restor Neurol Neurosci, 1998; 12:63-70; Yang et al., Carcinogenesis, 2000; 21:2035-203; Balasubramanian et al., J Biol Chem, 2002; 277:1828-1836; Saeki et al., Biochem J, 2002; 368:705-720). This apoptotic pathway also involves activator protein-1 (AP-1) inactivation (Dong, Biofactors, 2000; 12:17-28; Barthelman et al., Carcinogenesis, 1998; 19:2201-2204). Apoptosis induced by EGCG in certain in vitro cell models was reversed by exogenous catalase, suggesting H₂O₂ was the main cause for activation of the apoptotic pathway (Nakagawa et al., Biochem Biophys Res Commun, 2002; 292:94-101; Chai et al., Biochem Biophys Res Commun, 2003; 304:650-654).

It was also noted that while EGCG at low concentration (less than 10 μM) functions as a ROS scavenger, it functions as a ROS producer and can cause DNA damage at high concentrations (100 μM and above) (Saeki et al., Biochem J, 2002; 368:705-720). These observations lead to a hypothesis that GTPPs/EGCG-induced apoptosis under in vitro conditions is an artifact, especially when the GTPP or EGCG concentration is higher than the Cmax in the plasma (10 μM), since high levels of H₂O₂ cannot be achieved in vivo (Halliwell, FEBS Lett, 2003; 540:3-6).

However, it is not clear whether EGCG-induced apoptosis in tumor cells is indeed due to H₂O₂ generated in the culture medium or whether H₂O₂ is irrelevant to EGCG-induced responses when the EGCG concentration is at physiological levels (Dashwood et al., Biochemi Biophys Res Commun, 2002; 296:584-588). Thus, it is important to determine whether H₂O₂ generated under in vitro experimental conditions by EGCG at concentrations greater than 10 μM could be the driving force for tumor cell apoptosis (Hong et al., Cancer Res, 2002; 62:7241-7246).

This example demonstrated that EGCG-induced intracellular signaling (and the subsequent effects on the cell) depends upon the combination of many factors such as the concentration of EGCG, the origin of the cells, the culture media used, and the intracellular antioxidant enzymatic activity/quantity of the cell population. Therefore, H₂O₂ generated by EGCG might be a determinant factor for apoptosis in certain cell types and irrelevant in other cell types. As shown in Example 1, GTPPs/EGCG activate different pathways, depending on the cell type. EGCG at concentrations significantly higher than the Cmax found in the serum activates the survival pathway associated with terminal differentiation in normal epidermal keratinocytes, and the apoptotic pathway in oral carcinoma cells (Examples 3 and 5). Example 7 showed that EGCG in the 15-200 μM range reduced ROS/H₂O₂ to background levels in normal human primary epidermal keratinocytes (NHEK) and immortalized normal human salivary gland cells, while intracellular ROS/H₂O₂ levels were significantly elevated in oral carcinoma cells. This evidence suggests that high concentrations of EGCG could still be considered as physiological and clinical relevant for certain cells/tissues, since the digestive tract and the epidermis can be exposed to significant levels of GTPPs from the environment. Whether the key intracellular ROS scavenging enzymes catalase and superoxide dismutase (SOD) are differentially regulated by EGCG in normal versus tumor cells, or EGCG-induced cytotoxicity and growth arrest in tumor cells can be reversed by catalase or antioxidant are not clear. This example addressed these questions and compared the effects of EGCG with H₂O₂ in normal versus tumor cells.

Materials and Methods

Cell lines. NHEK were obtained from Cambrex Corporation (East Rutherford, N.J.) and maintained in KGM-2 medium (Cambrex Corporation). The OSC-2 and OSC-4 cell lines, which were isolated from cervical metastatic lymph nodes of patients with oral squamous cell carcinoma, as described in Example 6, were cultured in Dulbecco's Modified Eagle's Medium (DMEM)/Ham's F12 50/50 MIX medium (Cellgro, Kansas City, Mo.) supplemented with 10% (v/v) fetal bovine serum, 100 I.U/ml penicillin, 100 μg/ml streptomycin and 5 μg/ml hydrocortisone.

Reagents. Catalase, diamide, EGCG, H₂O₂, N-acetyl-L-cysteine (NAC), 3-amino-1,2,4-triazole (3-AT) and 3-(4, 5-dimethylthiazol-2-yl)-2, 5-diphenyl tetrazolium bromide (MTT) were purchased from Sigma-Aldrich (St. Louis, Mo.). Dihydrofluorescein diacetate (DFDA) and SOD were obtained from Molecular Probes Inc. (Eugene, Oreg.) and ICN Biomedicals Inc. (Aurora, Ohio), respectively.

Succinate dehydrogenase activity assay (MTT assay). This method directly detects the activity of mitochondrial succinate dehydrogenase (SDH). Change in SDH activity is a measurement of cell viability when stress is introduced in cell culture through chemical or physical means. In a 96-well microplate, 1.5×10⁴ cells were seeded in each well. After 24 hours treatment of EGCG at indicated doses, culture medium was removed and replaced with 100 μl of 2% MTT in a solution of 0.05 M Tris, 0.5 mM MgCl₂, 2.5 mM CoCl₂, and 0.25 M disodium succinate as substrate (Sigma, St. Louis, Mo.) and the plate was incubated at 37° C. for 30 minutes. Then 100 μl of 0.2 M Tris-HCl (pH 7.7) containing 4% (volume/volume (v/v)) formalin was added to each well and the microplate was incubated for 5 minutes at room temperature. After the incubation, the contents in each well were aspirated and each well was rinsed with 200 μl of H₂O followed by the addition of 100 μl dimethyl sulfoxide containing 6.25% (v/v) 0.1 N NaOH. Solubilized colored formazan product was measured using a Thermo MAX microplate reader (Molecular Devices Corp., Sunnyvale, Calif.) at a wavelength of 562 nm.

Measurement of intracellular ROS levels. The ROS assay (DFDA assay) measures the accumulation of intracellular ROS levels. The non-fluorescent dye dichlorofluorescein diacetate (DFDA) passively diffuses into cells, where the acetates are cleaved by intracellular esterases. The metabolites are trapped within the cells and oxidized by ROS, mainly hydrogen peroxide (H₂O₂), to the fluorescent form, 2′, 7′-dichlorofluorescein, which can be measured by a fluorescent plate reader to reflect levels of intracellular ROS (mainly H₂O₂). Thus, values of the fluorescence in the cell cultures are constantly rising in this assay due to the accumulation of ROS. Cells (1.5×10⁴ cells/well) were incubated with Hallam's physiological saline (HPS) containing DFDA (10 μM) in a 96-well microplate for 30 minutes at 37° C. After the incubation, cells were washed three times with HPS and then incubated with HPS containing EGCG (50-200 μM) or diamide (5 mM) for the indicated times. The intracellular ROS levels were measured by using a fluorescence plate reader (BIO-TEK FL600, Bio-Tek Instruments, Inc., Winooski, Vt.), at an excitation wavelength of 485 nm and an emission wavelength of 530 nm.

Caspase-3 activity assay. The caspase-3 apoptosis detection kit (Santa Cruz Biotechnology, Inc., Santa Cruz, Calif.) was used to measure caspase-3 activity. Cells (10⁵ cells/well) were plated in triplicate in a 24-well tissue culture plate. After 24 hours of treatment with EGCG, the cells in each well were washed with 1 ml of PBS and incubated with 100 μl of cell lysis buffer on ice for 10 minutes. To each well, 100 μl of 2×reaction buffer was added with 10 mM dithiothreitol. Finally, 5 μl of DEVD-AFC substrate was added to each well containing cell lysates. The reaction mixtures were incubated for 1 hour at 37° C., and caspase-3 activity in each well was measured using a fluorescence microplate reader (SPECTRAFluor Plus, Tecan US, Research Triangle Park, N.C.) at a wavelength of 405 nm for excitation and 505 nm for emission.

DNA synthesis assay. DNA synthesis was analyzed by a BrdU cell proliferation assay kit (Oncogene Research Products, Boston, Mass.). Briefly, cells (10⁴ cells/well) were seeded in a 96-well microplate and treated with the indicated doses of EGCG for 24 hours at 37° C. After the treatment, cells were labeled with BrdU for 2 hours at 37° C. and reacted with anti-BrdU antibody. Unbound antibody in each well was removed by rinsing, and horseradish peroxidase-conjugated goat anti-mouse IgG antibody was added to each well. The color reaction to visualize the secondary antibody was carried out according to the protocol provided by the manufacturer. The color reaction product was quantified using a Thermo MAX microplate reader (Molecular Devices Corp., Sunnyvale, Calif.) at dual wavelengths of 450-540 nm.

Western blotting. After EGCG-treatments, cells were washed in ice-cold PBS and lysed for 10 minutes in 1×PBS containing 1% (v/v) Nonidet P-40, 0.5% (w/v) sodium deoxycholate, 0.1% (w/v) SDS, 10 μg/ml leupeptin, 3 μg/ml aprotinin and 100 mM phenylmethylsulfonyl fluoride (PMSF). Samples of lysates containing 25 μg protein were loaded in each lane and electrophoretically separated on a 7.5% SDS polyacrylamide gel. Following electrophoresis, proteins were transferred to a nitrocellulose membrane (TRANS-BLOT Transfer Medium, Bio-Rad Laboratories, Hercules, Calif.). The membrane was blocked for 1 hour with 5% (w/v) non-fat dry milk powder in PBST (0.1% Tween-20 in PBS) and then incubated for 1 hour with anti-catalase rabbit polyclonal antibody (Abcam Ltd., Cambridge, United Kingdom), anti-manganese (Mn)-SOD rabbit polyclonal antibody (Upstate, Lake Placid, N.Y.) and anti-actin goat polyclonal antibody (Santa Cruz Biotechnology, Inc.). The membrane was washed three times with PBST and incubated with peroxidase-conjugated, affinity-purified anti-rabbit or anti-goat IgG (Santa Cruz Biotechnology, Inc.) for 1 hour. Following extensive washing, the reaction was developed by enhanced chemiluminescent staining using ECL Western blotting detection reagents (Amersham Pharmacia Biotech Inc., Piscataway, N.J.).

Assays for SOD and catalase activities. Cells (10⁵ cells/well) were incubated with or without EGCG (50 μM) in 24-well culture plates for desirable time periods at 37° C. After the incubation, cells were harvested and disrupted in 100 μl of 10 mM Tris-HCl (pH 7.4) containing 0.1% (v/v) Triton ×-100, 10 μg/ml leupeptin, 10 μg/ml pepstatin A and 100 mM PMSF by three cycles of freezing/thawing. After centrifugation at 17,000×g for 20 minutes at 4° C., the supernatants were used for SOD and catalase assays using the SOD Assay Kit-WST (Molecular Technologies, Inc., Gaithersburg, Md.) and the Amplex Red Catalase Assay Kit (Molecular Probes), respectively. The activities of SOD and catalase were calibrated using a standard curve prepared with purified human SOD and catalase. The activities of SOD and catalase were expressed as units (U)/10⁶ cells.

Statistical analysis. All data are reported as mean±SD. A one-way ANOVA and unpaired Student's t tests were used to analyze statistical significance. Differences were considered statistically significant at p<0.05.

Results and Discussion

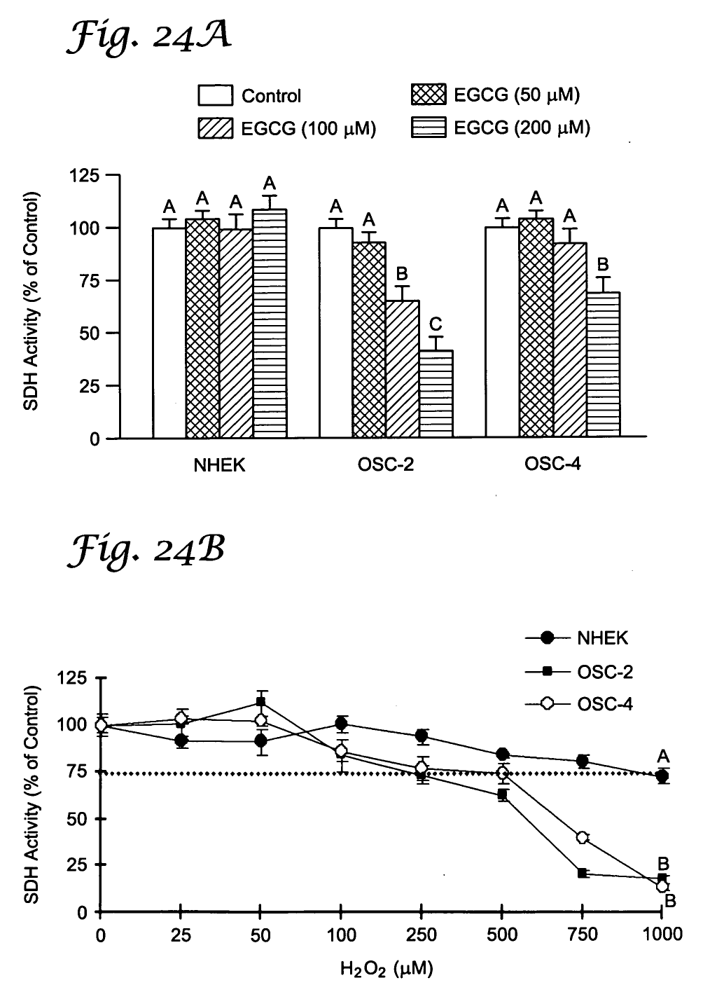

Susceptibility of NHEK and OSC cell lines to EGCG and H₂O₂. After 24 hours of incubation with EGCG at various concentrations, mitochondrial enzyme SDH activity (a measure of mitochondrial integrity) in NHEK was not altered (FIG. 24A). However, both OSC-2 and OSC-4 cells exhibited reduced SDH activities. OSC-2 was the more sensitive cell line, with SDH activity declining to less than 50% of untreated control levels after incubation with 200 μM EGCG (FIG. 24A). Unlike EGCG, H₂O₂ induced cytotoxicity in all cell types, with noticeable differences among the cell types. The SDH activities of all cell types gradually declined when H₂O₂ concentrations increased from 100 to 500 μM. At H₂O₂ concentrations higher than 500 μM, SDH activities in the OSC cell lines decreased more rapidly than in NHEK. Treatment with 1 mM H₂O₂ caused a 25% reduction of SDH activity in NHEK, but only 250 μM was needed to cause the same reduction in OSC-2 cells and OSC-4 cells. When these cells were exposed to 1 mM H₂O₂ for 24 hours, the SDH activities were reduced to less than 20% in both cell lines (FIG. 24B).

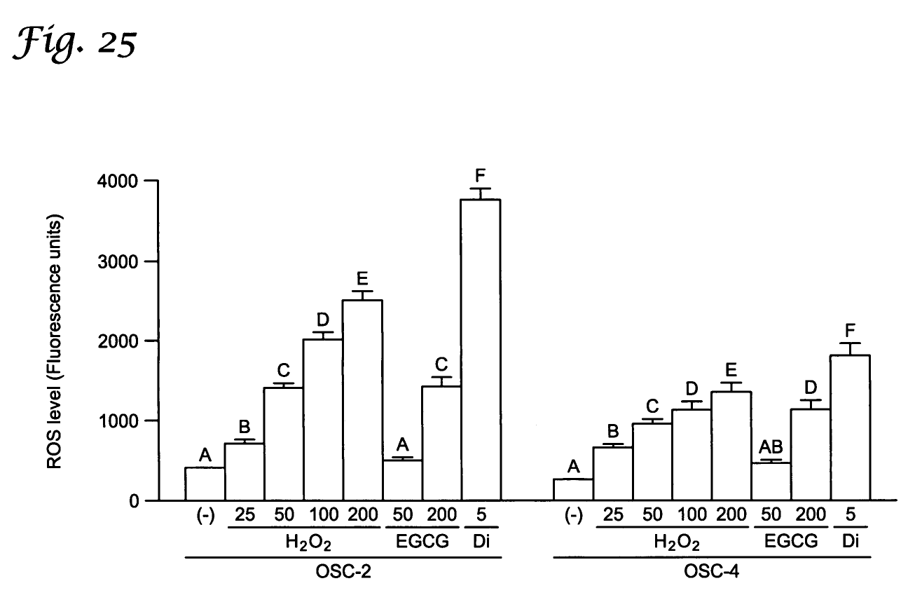

Generation of intracellular ROS by EGCG in comparison to exogenous H₂O₂ in OSC cell lines. As shown in Example 6, EGCG caused differential oxidative environments in normal versus tumor cells. EGCG at concentrations of 15 to 200 μM lowered ROS to background levels in NHEK. In contrast, the current study showed that, following a 60 minute exposure to either exogenous H₂O₂ or EGCG, both OSC-2 and OSC-4 cell lines exhibited a dose-dependent accumulation of intracellular ROS, as detected by dihydrofluorescein diacetate (DFDA) (FIG. 25). OSC-2 cells were more sensitive to diamide and H₂O₂ than OSC-4 cells. Under identical conditions, 5 mM diamide-induced ROS in OSC-2 cells was doubled that in OSC-4 cells, and doubled that of H₂O₂ at 100 or 200 μM (FIG. 25). Although relatively high levels of EGCG (200μM) induced ROS in both cell lines, the induced ROS levels were similar, and less than those induced by 100 μM H₂O₂. At low levels of EGCG (50 μM), intracellular ROS levels remained comparable to the controls, less than that produced by 25 μM H₂O₂ (FIG. 25).

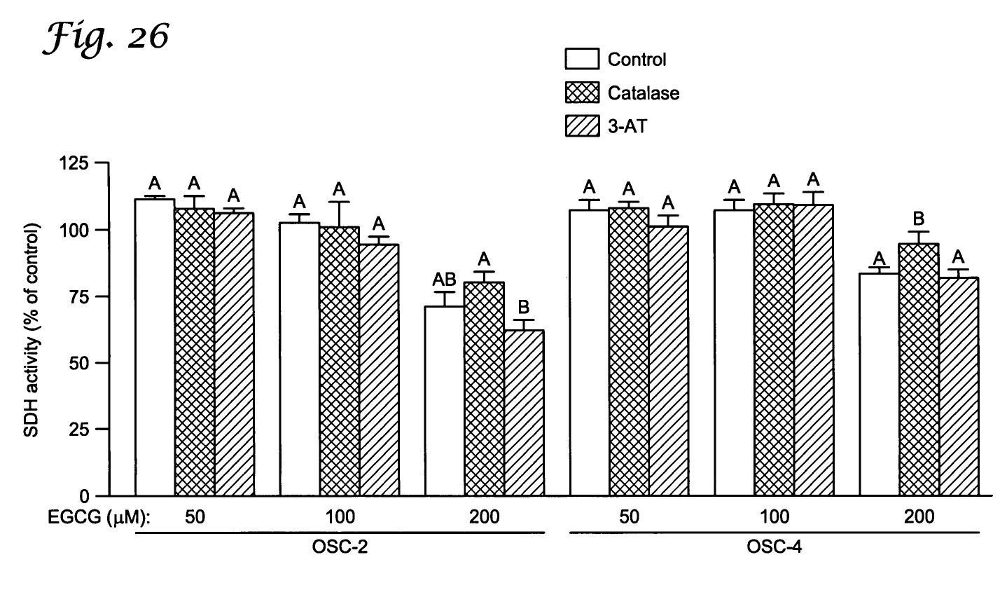

Impact of exogenous catalase and its inhibitor on OSC cell lines in response to EGCG. EGCG at 200 μM significantly reduced SDH activity in both OSC-2 and OSC-4 cell lines (FIG. 24A). Treatment with exogenous catalase had no effect on this reduction (FIG. 26). At this EGCG concentration, addition of a catalase inhibitor, 3-AT, had no significant effect on the decline of mitochondrial SDH activity in either OSC-2 or OSC-4 cells (FIG. 26). Treatment with exogenous catalase or 3-AT also failed to alter the effect of EGCG at concentrations of 50 and 100 μM in either cell line (FIG. 26). Moreover, NHEK did not become susceptible to EGCG cytotoxicity after pretreatment with 3-AT.

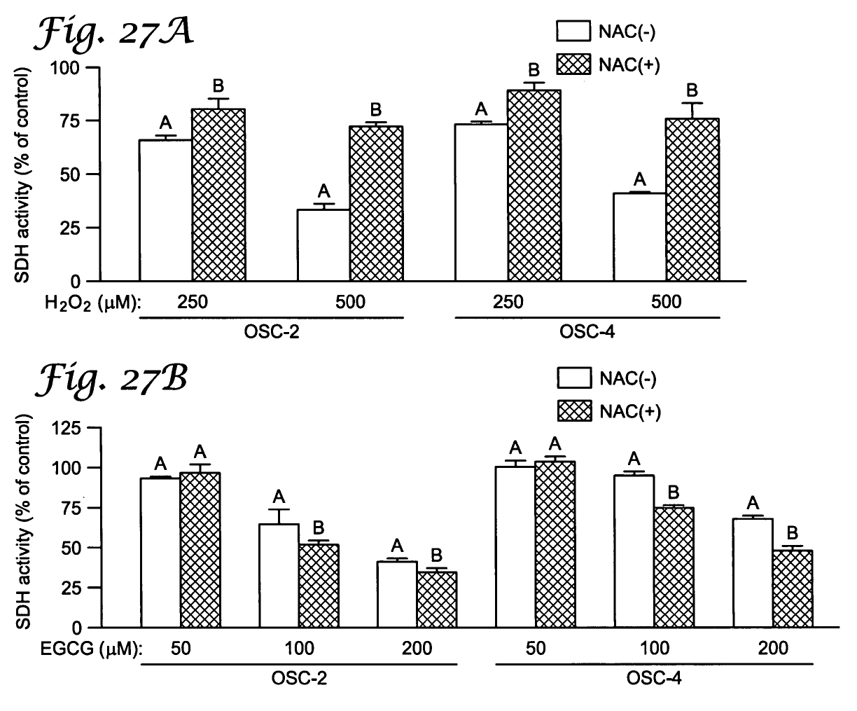

Comparison of the effect of EGCG with NAC in OSC cell lines. Two hour pretreatment with 10 mM NAC significantly inhibited the cytotoxic effect of H₂O₂ at 250 and 500 μM in OSC-2 and OSC-4 cell lines (FIG. 27A). However, NAC not only failed to rescue both cell lines from EGCG-induced cytotoxicity, but also enhanced the mitochondrial damage measured by MTT assays seen at higher EGCG levels (FIG. 27B).

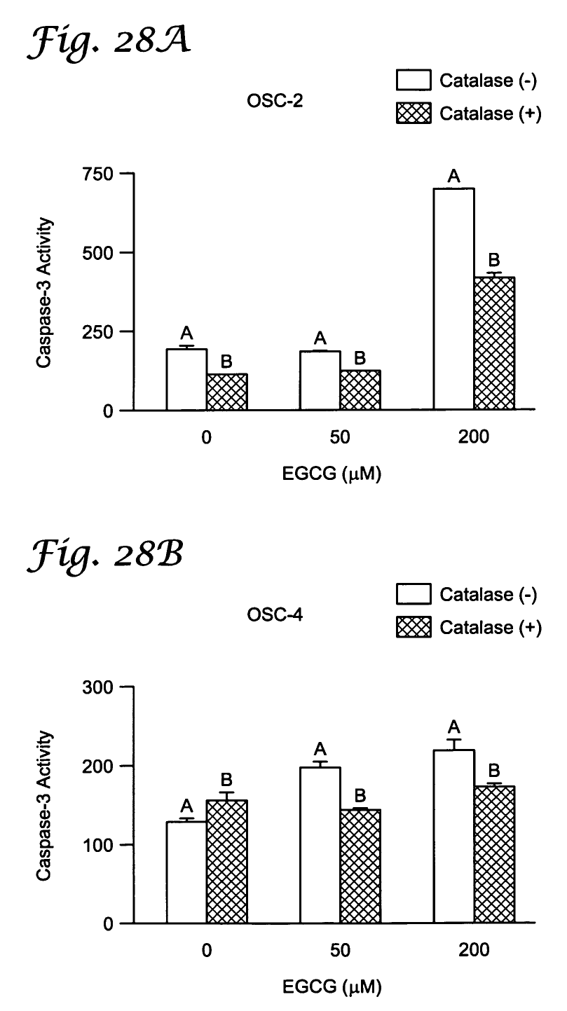

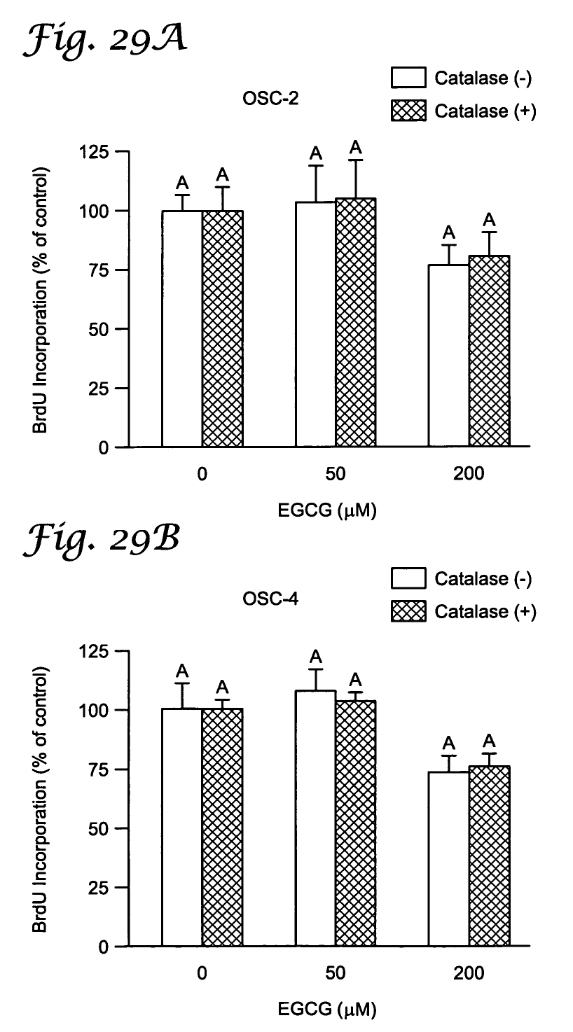

Impact of catalase on EGCG-induced tumor cell apoptosis and growth arrest. Exogenous catalase partially inhibited EGCG-induced caspase 3 activation in OSC-2 and OSC-4 cells during a 24 hour period (FIG. 28). However, although EGCG at 200 μM reduced BrdU incorporation by approximately 25% in both OSC-2 and OSC-4 cell lines within a 24-hour period, addition of exogenous catalase had no effect on the rates of BrdU incorporation (FIG. 29).

Levels of activity and quantity of endogenous catalase and SOD in response to EGCG exposure. When enzymatic activities were compared among these cells, NHEK had the highest levels of catalase activity, twice that found in OSC-4, and triple that in OSC-2 cells (FIG. 29A). However, OSC-2 cells exhibited the highest levels of total SOD activity, double those found in either NHEK or OSC-4 cells (FIG. 29A). EGCG had no effect on the enzymatic activity levels during the 24-hour treatment period, except for the catalase activity in OSC-4 cells, which showed a slight decrease (FIG. 29B). Of the three cell types, OSC-2 cells possess the lowest amount of endogenous catalase protein as compared to NHEK and OSC-4 cells, and the highest levels of Mn-SOD protein levels, consistent with the activity levels. Significant alteration in the protein levels of these enzymes was not observed during the 24-hour period following EGCG treatment (FIG. 29B). When exposed to EGCG, NHEK showed a slight decrease in catalase protein level and an increase in Mn-SOD protein at the 24-hour time point (FIG. 29B).

As previous in vitro studies demonstrated, EGCG induces differential effects in normal versus tumor cells, including 1) induction of growth arrest, regulation of MAP kinase pathway, accumulation of intracellular ROS, cytochrome c release, inhibition of AP-1and nuclear factor κB (NFκB), activation of caspase cascade, inhibition of cell invasiveness and induction of apoptosis in many tumor cells systems (2) activation of AP-1, induction of p57 and caspase 14 (a terminal differentiation marker for epidermal keratinocytes), reduced intracellular ROS, cell differentiation, elevated mitochondrial SDH activity (in aged keratinocytes), inhibition of p21 expression and stimulation of MAP kinase pathway (see Examples 1, 3, and 5). Based on these observations, the roles of H₂O₂ and endogenous antioxidant enzymes in EGCG-induced effects are unlikely to be the same among different cell types from various origins. For example, as shown in Example 6, EGCG elevated ROS, especially H₂O₂ levels in tumor cells but not NHEK or immortalized normal salivary gland cells, which correlated with either apoptotic or survival pathways. In addition, elimination of H₂O₂ by addition of catalase could not prevent EGCG-induced inhibition of AP-1 and activation of JNK and ERK, suggesting EGCG signaling might not solely rely on oxidative stress (Chung et al., Cancer Res, 1999; 59:4610-4617). The current study further confirmed that high concentrations of EGCG damaged only tumor cells (OSC-2 and OSC-4), but not normal cells (NHEK) (FIG. 24A). Importantly, the EGCG-induced differential effect in normal versus tumor cells could not be reproduced entirely by H₂O₂ alone (FIG. 24B), and the damage imposed on NHEK by H₂O₂ was less severe than that on the OSC cells. Both OSC cell lines showed a significant decline in mitochondrial SDH activity at H₂O₂ concentrations of 250 μM or more, and the SDH activities were reduced to less than 25% of control levels when the H₂O₂ concentration was increased to 1 mM (FIG. 24B). In comparison, 75% of SDH activity remained in NHEK when treated with 1 mM H₂O₂ for 24 hours (FIG. 24B). These results demonstrated that NHEK possess a stronger ability to resist the oxidative stress from H₂O₂, while OSC cells are more sensitive to H₂O₂-induced cytotoxicity. In comparison, EGCG at various concentrations did not induce cytotoxicity in NHEK, suggesting that H₂O₂-induced effects among these cell types are quantitative, but EGCG-induced effects among these cells are qualitative.

Between the tumor cell lines, OSC-2 cells appeared to be more sensitive to H₂O₂-induced cytotoxicity than OSC-4 cells, as measured by SDH activity (FIG. 24B). Consistent with this, when OSC-2 and OSC-4 cells were incubated with relatively high concentrations of H₂O₂ or diamide, OSC-2 cells accumulated significantly higher (approximately two-fold) ROS than OSC-4 cells, indicating that OSC-2 cells possess weaker defenses against H₂O₂ (FIG. 25). In OSC-2 cells, incubation with 200 μM EGCG produced ROS equivalent to that from 50 μM H₂O₂ during the first hour (FIG. 25). The SDH activity was reduced to 40% of untreated control after 24 hours (FIG. 24A). In contrast, 24 hour treatment with 50 μM H₂O₂ had no effect on the SDH activity (FIG. 24B). Similarly, incubation of OSC-4 cells with 200 μM EGCG produced ROS equivalent to that from 100 μM H₂O₂ during the first hour (FIG. 25), and the SDH activity was reduced to less than 75% of untreated control after 24 hours (FIG. 24A), but 100 μM H₂O₂ had no significant effect on SDH activity (FIG. 24B). Further discordance between the effects of H₂O₂ and EGCG was seen using reagents that directly or indirectly affect the H₂O₂ concentration. Neither exogenous catalase nor the addition of catalase inhibitor 3-AT had any major effect on the EGCG-induced SDH reduction in OSC cells (FIG. 26). A modest exception was seen in OSC-4 cells, where catalase partially reversed the effects of 200 μM EGCG. In addition, the strong antioxidant NAC not only failed to rescue the OSC cells from EGCG-induced SDH inhibition, it enhanced the EGCG-induced cytotoxicity, especially in OSC-4 cells, whereas it significantly rescued the OSC cells from H₂O₂-induced SDH inhibition (FIG. 27). Therefore, the cytotoxicity induced by EGCG in these tumor cells, as measured by mitochondrial damage, did not correlate with the ability of EGCG to produce ROS.

EGCG-induced growth arrest also appeared to not be strongly dependent on ROS production. 200 μM EGCG decreased BrdU incorporation to approximately 75% of control levels in both tumor cell lines, and exogenous catalase had no effect on the inhibition of DNA synthesis (FIG. 29).

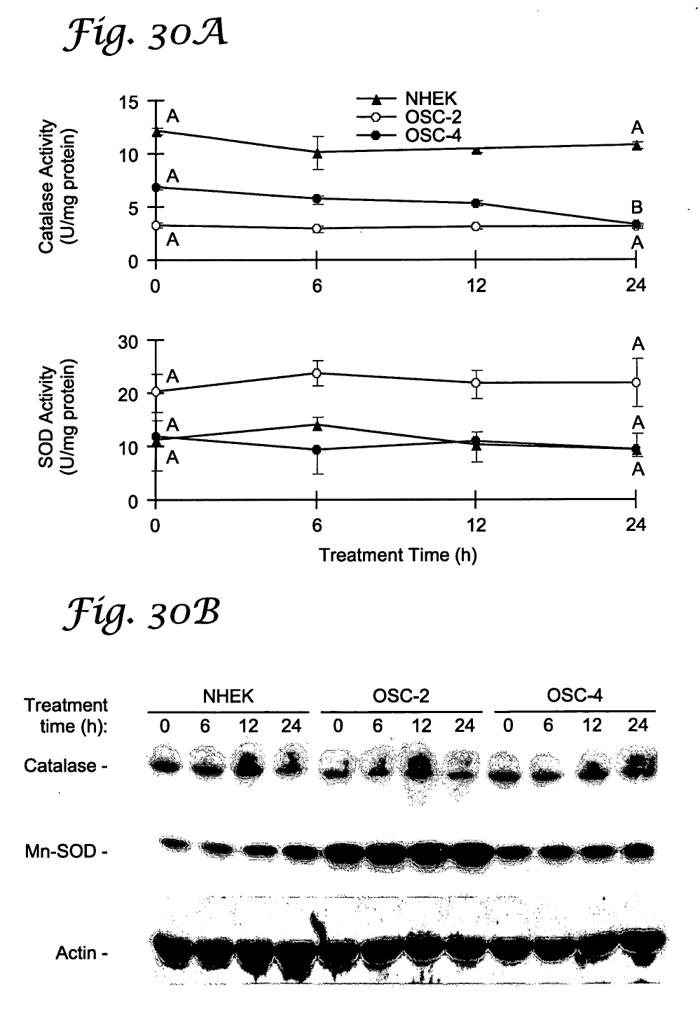

In contrast to the above observations regarding mitochondrial damage and growth arrest, EGCG-derived ROS do appear to have a role in caspase-3 activation. Exogenous catalase partially rescued OSC-2 cells, and substantially rescued OSC-4 cells from EGCG-induced caspase-3 activation during a 24 hour period (FIG. 28). Further, the levels of endogenous catalase activity are inversely correlated with sensitivity to EGCG, H₂O₂ and diamide (FIG. 30A, FIG. 24 and FIG. 25). SOD is unlikely to be involved, since there is no correlation between endogenous total SOD activity and cell sensitivity to EGCG, H₂O₂ or diamide (FIG. 30B, FIG. 24 and FIG. 25). In fact, OSC-2 cells, which showed high sensitivity to EGCG, diamide and H₂O₂, have the highest levels of Mn-SOD expression (FIG. 30B) and total SOD activity (FIG. 30A). The above observations are unlikely to be the result of an effect of EGCG on enzymes involved in ROS breakdown in OSC cells. EGCG did not appear to regulate markedly either catalase or SOD enzymatic activities or protein levels over a 24 hour period (FIG. 30).

In conclusion, EGCG-induced ROS formation is not simply concentration dependent, but is also cell type dependent. Identical concentrations of EGCG (as high as 200 μM) may cause severe damage in one tumor cell line (OSC-2), a less severe damage in another tumor cell line (OSC-4), but reduce ROS levels in a normal epithelial cells (NHEK). The data obtained in this example indicates that cells in potentially frequent contact with plant-derived polyphenols, such as cells found in the epidermis, oral mucosa and digestive tract, have developed mechanism(s) to mitigate cytotoxicity otherwise caused by the polyphenols and benefit from these compounds. However, EGCG, when applied in high doses, is cytotoxic to other human cells that lack this tolerance and to cancer cells that have lost these protective mechanisms. Thus, whether an EGCG concentration is “physiological relevant” or “clinically relevant” is organ/tissue dependent. In NHEK, EGCG induces a survival pathway associated with differentiation that does not appear to involve ROS. In OSC cells, EGCG induces different pathways that lead to cell death. Caspase-3 activation appears to involve EGCG-induced ROS formation, while mitochondrial damage and growth arrest do not. Endogenous catalase plays an role in a cell's response to EGCG, cells without adequate catalase are more sensitive to EGCG-induced H₂O₂ formation as shown in the current study and previous reports (Yang et al., Carcinogenesis, 1998; 19:611-616; Sakagami et al., Anticancer Res, 2001; 21:2633-2641; Chai et al., Biochem Biophys Res Commun, 2003; 304:650-654). However, H₂O₂ alone cannot reproduce the EGCG effects in other cell lines or cell types. Thus, applications of high concentrations of EGCG on epithelial tissues, especially the epidermal and digestive tract tissues, for chemoprevention purposes could deliver cytotoxic effects involving growth arrest/apoptosis signaling and oxidative stress that are clinically relevant, while normal epidermal cells are guided to safety by a cell differentiation pathway.

### Example 10

### Macroarray Analysis of Tea Polyphenol-Treated Normal Versus Malignant Epithelial Cells

The most abundant polyphenol in green tea, (−)-epigallocatechin gallate (EGCG), has anti-tumor effects. Whereas tumor cells undergo apoptosis after exposure to EGCG, normal epithelial cells do not. Apoptosis macroarrays were used to examine both normal and metastatic oral carcinoma cells for intracellular target(s) of EGCG.

Normal human epidermal keratinocytes (NHEK; Cambrex), and oral squamous cell carcinoma (OSC2) cells, originally from gingival tissue, were compared. Exponentially growing cells were exposed to 50 or 100 μM EGCG for 0 hours, 2 hours, 6 hours or 24 hours. Cells were harvested for total RNA, which was examined by apoptosis macroarrays (Sigma Genosys), followed by phosphorimagery and data analysis (GeneSpring). Protein production of expressed genes was confirmed by Western blotting of whole cell lysates.

Following treatment of NHEK with 100 μM EGCG, only caspase 14 gene expression was upregulated by at least 2-fold. Production of immunoreactive caspase 14, an epithelial cell-specific protein involved in epidermal cell terminal differentiation, was increased even further (5-fold) by lower doses of EGCG (30 μM). Numerous NHEK genes were significantly down-regulated over 24 hours, including: a) cell cycle regulators, such as p53, p21, and c-myc; b) several apoptosis-related genes, including cytochrome C, cyclooxgenase-2, and glyceraldehyde 3-phosphate dehydrogenase; and c) the Bcl-2 related genes, Bcl-x and Mcl-1.

In contrast, OSC2 cells expressed early increases in Mcl-1 and cyclin D, and p21 mRNA was elevated 3-fold within 2 hours of EGCG exposure. Expression of p53 transiently decreased, between 2 and 6 hours, but returned to baseline by 24 hours.

EGCG has opposite effects on the expression of a number of genes that direct apoptosis and/or cell division in normal (NHEK) versus OSC2 cells. Exploration of these divergent EGCG-responsive pathways in epithelial cells is under way.

### Example 11

### Chemopreventive Effects of Green Tea Polyphenol Is Associated With Caspase 14 Induction in Epidermal Keratinocytes

A unique feature related to the chemopreventive effects of green tea polyphenols (GTPP) is that these compounds induce apoptosis in tumor cells while inducing differentiation in normal epithelial cells. (−)-Epigallocatechin-3-gallate (EGCG), the most abundant GTPP, specifically induces the expression of p57, a cyclin dependent kinase inhibitor that plays an important role in cell growth and differentiation. Induction of p57 is required for cell survival when cells are exposed to EGCG. It has been shown that EGCG-induced epidermal keratinocyte differentiation blocks these cells from undergoing caspase-mediated apoptosis. The purpose of the current study is to investigate whether caspase 14, a caspase family member that is specifically involved in epidermal cell terminal differentiation, also participates in the EGCG-induced keratinocyte differentiation. Results of RT-PCR, immunoblot, immunocytochemistry and gene array techniques with pooled primary normal human epidermal keratinocytes (NHEK) with or without EGCG exposure indicate that caspase 14 is induced by EGCG subsequent to p57 induction. Therefore, it appears that EGCG-induced NHEK differentiation is associated with caspase 14 induction, possibly mediated by p57 action. In conclusion, the ability of EGCG to potently accelerate epidermal differentiation could be applied for treatment of selected epithelial disorders, including pre-cancerous lesions in the epidermis and the oral cavity. In addition, the differentiation-inducing potential of p57/caspase 14 can be applied for cancer therapy.

### Example 12

### Protection of Salivary Gland Cells Against Xerostomia by Green Tea Protective Role of Green Tea Polyphenol in Salivary Gland Cells versus Oral Cancer Cells Under Therapeutic Conditions

Xerostomia, resulting from destruction of salivary gland cells, is often associated with chemotherapy and radiation therapies among oral cancer patients. The major green tea polyphenol, -(−) epigallocatechin-3-gallate (EGCG), has been found to simultaneously protect normal epithelial cells from reactive oxygen species, and induce apoptosis in tumor cells.

The goal of the current study is to investigate whether EGCG protects normal salivary gland cells from chemotherapy drug cisplatin (CDDP, cis-[PtCl₂(NH₃)₂]) and ultraviolet irradiation at wavelength of 254 nm-induced cytotoxicity, and enhance the therapeutic effect on salivary gland tumor cells.

Human immortalized salivary acenar cells (AC) and duct cells (DC), along with human salivary gland tumor cells (HSG, a radiation-resistant cell line) and oral squamous carcinoma cells (OSC) were either treated by CDDP or irradiated by UVC with or without the presence of EGCG, followed by determination of the mitochondrial succinate dehydrogenase (SDH) activity, a measurement of mitochondrial damage and BrdU incorporation determination.

The result demonstrated that pretreatment of EGCG significantly protected the normal salivary gland cells from CDDP and UVC, but not the tumor cells. EGCG may be applicable in chemotherapy and/or radiation therapy to protect normal salivary tissue and simultaneously induce tumor cell apoptosis.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

What is claimed is:

1. A method of determining if cancer cells are resistant to an agent, the method comprising:
determining the p57/KIP2 level in the cancer cells prior to contact with the agent;
contacting the cancer cells with the agent;
determining the p57/KIP2 level in the cancer cells after contact with the agent; and
comparing the p57/KIP2 level in the cancer cells after contact with the agent to the p57/KIP2 level in the cancer cells prior to contact with the agent;
wherein an increase in the p57/KIP2 level in the cancer cells after contact with the agent compared to the p57/KIP2 level in the cancer cells prior to contact with the agent indicates the cancer cells are resistant to the agent.

2. The method of claim 1, wherein the cancer cell is an epithelial carcinoma cell line.

3. The method of claim 2, wherein the epithelial carcinoma cell lines is selected from the group consisting of an oral squamous carcinoma cell line, a metastatic oral carcinoma cell line, and a breast epithelial carcinoma cell line.

4. The method of claim 1, wherein the cancer cells are derived from a human epithelial carcinoma.

5. The method of claim 4, wherein the human epithelial carcinoma is selected from the group consisting of an oral squamous carcinoma, a metastatic oral carcinoma, and a breast epithelial carcinoma.

6. The method of claim 1, wherein determining the p57/KIP2 level is by detecting the p57/KIP2 protein.

7. The method of claim 1, wherein determining the p57/KIP2 level is by detecting the mRNA encoding p57/KIP2.

8. A method of determining if cancer cells are sensitive to an agent, the method comprising:
determining the p57/KIP2 level in the cancer cells prior to contact with the agent;
contacting the cancer cells with the agent;
determining the p57/KIP2 level in the cancer cells after contact with the agent; and
comparing the p57/KIP2 level in the cancer cells after contact with the agent to the p57/KIP2 level in the cancer cells prior to contact with the agent;
wherein no increase in the p57/KIP2 level in the cancer cells after contact with the agent compared to the p57/KIP2 levels in the cancer cells prior to contact with the agent indicates the cancer cells are sensitive to the agent.

9. A method of identifying an agent effective for the treatment of a cancer, the method comprising;
determining the p57/KIP2 level in cancer cells prior to contacting with the agent;
contacting the cancer cells with the agent;
determining the p57/KIP2 level in the cancer cells after contacting with the agent; and
comparing the p57/KIP2 level in the cancer cells after contacting with the agent to the p57/KIP2 level in the cancer cells prior to contacting with the agent;
wherein no increase in the p57/KIP2 level in the cancer cells after contacting with the agent compared to the p57/KIP2 level in the cancer cells prior to contacting with the agent indicates the agent is effective for the treatment of a cancer.

10. A method of determining the therapeutic effectiveness of an agent, the method comprising:
contacting normal cells with the agent;
determining the p57/KIP2 level in the normal cells after contacting with the agent;
contacting cancer cells with the agent;
determining the p57/KIP2 level in the cancer cells after contacting with the agent; and
comparing the p57/KIP2 level in the normal cells after contacting with the agent to the p57/KIP2 level in the cancer cells after contacting with the agent;
wherein a higher p57/KIP2 level in the normal cells compared to the p57/KIP2 level in the cancer cells indicates the agent is effective for the treatment of cancer.

11. The method of claim 10, wherein the normal cells and cancer cells are cultured together.

12. A method of optimizing the formulation of an agent for the treatment of a cancer, the method comprising:
contacting cancer cells with a first formulation of the agent;
determining the p57/KIP2 level in the cancer cells contacted with the first formulation of the agent;
contacting cancer cells with a second formulation of the agent;
determining the p57/KIP2 level in the cancer cells contacted with the second formulation of the agent; and
comparing the p57/KIP2 level in the cancer cells contacted with the first formulation of the agent to the p57/KIP2 level in the cancer cells contacted with the second formulation of the agent;
wherein the formulation with the lower level of p57/KIP2 indicates the formulation of the agent more effective for the treatment of a cancer.

13. A method of preventing damage to non-cancerous cells in a subject undergoing cancer therapy, the method comprising administering to the subject a polyphenolic composition under conditions effective to induce the expression of p57, induce the expression of caspase-14, or induce the expression of both p57 and caspase-14 in non-cancerous cells.

14. The method of claim 13 wherein the polyphenolic composition is selected from the group consisting of green tea polyphenol (GTPP), (−)-epicatechin (EC), (−)-epigallocatechin (EGC), (−)-epicatechin-3-gallate (ECG) and (−)-epigallocatechin-3-gallate (EGCG), and combinations thereof.

15. The method of claim 14 wherein the polyphenolic composition comprises EGCG.

16. The method of claim 13 wherein the polyphenolic composition is administered to the subject prior to, coincident with, or subsequent to the cancer therapy.

17. The method of claim 13, wherein the cancer is selected from the group consisting of oral cancer, esophageal cancer, gastric cancer, colorectal cancer, prostate cancer, bladder cancer, skin cancer, and cervical cancer.

18. The method of claim 13, wherein the cancer therapy is selected from the group consisting of chemotherapy, radiation therapy, and a combination thereof.

19. A method of enhancing the effectiveness of a cancer therapy in a subject undergoing cancer therapy, the method comprising administering to the subject a polyphenolic composition under conditions effective to induce caspase 3-dependent apoptosis in cancer cells.

20. A method of preventing damage to salivary glands cells in a subject undergoing therapy for oral cancer, the method comprising administering to the subject a polyphenolic composition under conditions effective to induce the expression of p57, induce the expression of caspase-14, or induce the expression of both p57 and caspase-14 in the salivary gland cells.

21. A method of treating a skin condition comprising contacting the skin with a polyphenolic composition under conditions effective to induce caspase-14 expression in keratinocytes.

22. The method of claim 21, wherein the skin condition is selected from the group consisting of psoriasis, aphthous ulcer, actinic keratosis, rosacea, a wound, a burn, a skin condition associated with diabetes, a skin condition associated with aging, and a skin condition associated with altered keratinocyte differentiation.

23. A method of treating a precancerous oral lesion comprising contacting the precancerous oral lesion with a polyphenolic composition under conditions effective to induce p57 expression in normal epithelial cells and induce caspase 3-dependent apoptosis in precancerous and cancerous epithelial cells.

24. An in vitro method for the identification of an agent that possesses both a cytotoxic effect on tumor cells and a protective effect on normal cells, the method comprising:
co-culturing normal cells adjacent to tumor cells in vitro;
contacting the co-cultured cells with an agent;
determining if contact with the agent induces tumor cell death; and
determining if normal cells survive upon contact with the agent; and
wherein the induction of tumor cell death by contact with the agent and the survival of normal cells upon contact with the agent indicated the agent possesses both a cytotoxic effect on tumor cells and a protective effect on normal cells.

25. The method of claim 24, wherein both the tumor cells and normal cells are of epithelial origin.

26. The method of claim 24, wherein both the tumor cells and normal cells are human cells.

27. The method of claim 24, wherein the induction of tumor cell death upon contact with an agent is determined by detecting apoptosis of the tumor cell.

28. The method of claim 27, wherein the tumor cells are a tumor cell line stably transfected with green fluorescent protein (GFP).

29. The method of claim 28, wherein the tumor cell line stably transfected with GFP is the human oral carcinoma cell line OSC-2.

30. The method of claim 24, wherein survival of normal cells upon contact with an agent is determined by detecting the induction of p57 expression in the normal cells.

31. The method of claim 30, wherein the induction of expression of p57 is determined by detecting the p57 protein.

32. The method of claim 30, wherein the induction of expression of p57 is determined by detecting the mRNA encoding the p57 protein.

33. The method of claim 30, wherein the normal cells are normal human primary epidermal keratinocytes or fibroblasts.

34. An agent identified by the method of claim 24.

35. A kit for the identification of an agent that possesses both a cytotoxic effect on tumor cells and a protective effect on normal cells, the kit comprising normal cells, tumor cells transfected with green fluorescent protein (GFP), and printed instructions for the identification of an agent that possesses both a cytotoxic effect on tumor cells and a protective effect on normal cells.
